(19) United States
(12) Patent Application Publication
(10) Pub. No.: US 2015/0031600 A1
(43) Pub. Date: Jan. 29, 2015

(54) DESIGNED BIOSURFACTANTS, THEIR MANUFACTURE, PURIFICATION AND USE

(71) Applicants: Anton Peter Jacob Middelberg, Queensland (AU); Mirjana Dimitrijev-Dwyer, Queensland (AU); Michael Brech, Christchurch (NZ)

(72) Inventors: Anton Peter Jacob Middelberg, Queensland (AU); Mirjana Dimitrijev-Dwyer, Queensland (AU); Michael Brech, Christchurch (NZ)

(73) Assignee: The University of Queensland, Queensland (AU)

(21) Appl. No.: 13/994,482

(22) PCT Filed: Dec. 14, 2011
(86) PCT No.: PCT/AU2011/001619

§ 371 Date: Aug. 27, 2013

(30) Foreign Application Priority Data
Dec. 14, 2010 (AU) 2010905481
Sep. 29, 2011 (AU) 2011904003

(51) Int. Cl. C11D 3/386; C07K 14/00

(52) U.S. Cl. CPC C11D 3/386; C07K 14/001
USPC 514/2.3; 530/324; 530/325; 510/392

(57) ABSTRACT

The present invention relates to designed polypeptide biosurfactants that may be prepared by recombinant technology in commercially useful amounts and purified by simple non-chromatographic methods. The designed polypeptide biosurfactants comprise at least one stimuli-responsive amino acid residue or at least one glutamine or asparagine residue and may be useful in modulating the stability of a foam, alone or in combination with an α-helical peptide. The designed polypeptide biosurfactant may be useful in the formation and collapse of foams in foods, beverages, pharmaceuticals, personal care products, cosmetics, cleaning products, mineral recovery, bioremediation, oil recovery and laundry products. The designed biosurfactants may also be useful in recombinant production and purification of peptides, polypeptides and proteins.

### FIELD OF THE INVENTION

The present invention relates to designed protein and polypeptide biosurfactants that may be prepared by recombinant technology in commercially useful amounts. Methods of purifying the biosurfactants and their use are also described. In particular aspects of the invention, the biosurfactants are useful in switchable foam control.

### BACKGROUND OF THE INVENTION

Foams are metastable dispersions of gas in a liquid matrix often stabilized by surfactant adsorption at the air-water interface. They find application in industrial sectors including household and personal care, food, and environmental, oil and mineral processing. In products such as beer consumers desire stable foam while in other applications such as cleaning or processing, foaming is controlled by the addition of specific agents or by mechanical breakage.

Biosurfactants are increasingly viewed as renewable products and can be classified as lipopeptide, peptide amphiphile, protein hydrolysate or designed peptide surfactants. Of these, designed peptide surfactants provide a high level of foam control through the formation and dissipation of supramolecular structure at the air-water interface. Although offering a high level of foam control, peptide-based supramolecular chemistry currently remains too costly for broad application in low-cost industrial sectors.

There is a need for designed polypeptide and protein biosurfactants that can be manipulated in a controlled manner and manufactured in commercially viable amounts with simple and cost effective product release, recovery and purification.

### SUMMARY OF THE INVENTION

The present invention is predicated in part on the discovery that polypeptides and proteins that fold into helix bundles can be designed to provide a biosurfactant that has stimuli-responsive properties allowing control of foam stability and may also be manufactured in commercially viable amounts and purified by simple low cost techniques.

According to one aspect of the present invention there is provided a polypeptide or protein comprising at least two α-helical peptides linked by a linking sequence of 3 to 11 amino acid residues, wherein the biosurfactant has a folded tertiary structure with a hydrophobic core and a hydrophilic surface; and wherein each α-helical peptide comprises a sequence of amino acid residues:
(a b c d d′ e f g)ₙ
   wherein n is an integer from 2 to 12;
   amino acid residues a and d are hydrophobic amino acid residues;
   amino acid residue d′ is absent or is a hydrophobic amino acid residue;
   at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
   amino acid residue g is any amino acid residue;
   wherein
i) each α-helical peptide comprises at least one stimuli-responsive amino acid residue; or
ii) each sequence (a b c d d′ e f g) in the α-helical peptide comprises at least one glutamine or asparagine residue and no net charge or each sequence (a b c d d′ e f g) comprises at least one glutamine or asparagine residue and one negative charge.

In some embodiments the at least one stimuli-responsive amino acid residue is a lysine residue. In some embodiments the at least one stimuli-responsive amino acid residue is a histidine residue. In some embodiments the at least one stimuli-responsive amino acid residue results from each sequence (a b c d d′ e f g) in the α-helical peptide having a net negative or positive charge at a specified pH.

In some embodiments, each sequence (a b c d d′ e f g) in the α-helical peptide comprises at least two glutamine or asparagine residues and no net charge. In some embodiments, each sequence (a b c d d′ e f g) in the α-helical peptide comprises at least one glutamine or asparagine residue and one negative charge.

In some embodiments, the α-helical peptide comprises at least one charged amino acid residue.

In some embodiments, the linking sequence has 3 to 5 amino acid residues, especially 3 amino acid residues. In some embodiments, the linking sequence comprises a cleavable bond, especially an acid cleavable bond, especially a D-P bond. In some embodiments, the linking sequence comprises the sequence D-P-S. In some embodiments, the linking sequence is D-P-S.

In a further aspect of the invention there is provided an α-helical peptide comprising the amino acid sequence:
X₁-(a b c d d′e f g)ₙ-X₂
   wherein n is an integer from 2 to 12;
   amino acid residues a and d are hydrophobic amino acid residues;
   amino acid residue d′ is absent or is a hydrophobic amino acid residue;
   at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
   amino acid residue g is any amino acid residue;
   wherein
i) each α-helical peptide comprises at least one stimuli-responsive amino acid residue; or
ii) each sequence (a b c d d′ e f g) in the α-helical peptide comprises at least one glutamine or asparagine residue and no net charge or each sequence (a b c d d′ e f g) comprises at least one glutamine or asparagine residue and one negative charge; and
   X₁ and X₂ are each independently absent or are amino acid residues from a cleavable linking sequence of the polypeptide or protein of the invention and wherein the number of amino acid residues X₁+X₂ is 0 to 11 amino acid residues.

In some embodiments the at least one stimuli-responsive amino acid residue is a lysine residue. In some embodiments the at least one stimuli-responsive amino acid residue is a histidine residue. In some embodiments the at least one stimuli-responsive amino acid residue results from each sequence (a b c d d′ e f g) in the α-helical peptide having a net negative or positive charge at a specified pH.

In some embodiments, each sequence (a b c d d′ e f g) in the α-helical peptide comprises at least two glutamine or asparagine residues and no net charge. In some embodiments, each sequence (a b c d d′ e f g) in the α-helical peptide comprises at least one glutamine or asparagine residue and one negative charge.

In some embodiments, the α-helical peptide comprises at least one charged amino acid residue.

In another aspect of the invention there is provided a method of modulating the stability of foam comprising a polypeptide or protein biosurfactant at a liquid-gas interface; wherein said biosurfactant comprises at least two α-helical peptides linked by a linking sequence of 3 to 11 amino acid residues, and wherein each α-helical peptide comprises a sequence of amino acid residues:
(a b c d d′ e f g)ₙ
   wherein n is an integer from 2 to 12;
   amino acid residues a and d are hydrophobic amino acid residues;
   amino acid residue d′ is absent or is a hydrophobic amino acid residue;
   at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
   amino acid residue g is any amino acid residue;
   and wherein each α-helical peptide comprises a stimuli-responsive amino acid residue;
   said method comprising the step of:
i) exposing the biosurfactant to a stimulus that alters the zeta potential and/or surface charge of the biosurfactant at the liquid-gas interface or the metal ion binding of the biosurfactant or hydration structure of the biosurfactant at the liquid-gas interface.

In some embodiments, the α-helical peptide comprises a lysine residue. In these embodiments, the stimulus alters the surface charge at the liquid-gas interface, for example, by altering the pH of the biosurfactant. In some embodiments the stimulus is an acid. In other embodiments, the stimulus is a base. In yet other embodiments, pH and therefore the surface charge of the liquid-gas interface may be altered by dilution of bulk aqueous phase from which the foam is formed.

In some embodiments, the α-helical peptide comprises a histidine residue. In these embodiments, the stimulus alters the metal ion binding of the biosurfactant. In some embodiments, the stimulus is a metal ion or a chelating agent.

In some embodiments, each sequence (a b c d d′ e f g) in the α-helical peptide has a net negative or positive charge at a specified pH. In these embodiments, the liquid-gas interface comprising the biosurfactant also has a net negative and/or positive charge and the stimulus alters the hydration structure of the biosurfactant at the liquid-gas interface. In some embodiments, the stimulus is a kosmotropic or chaotropic salt.

In other embodiments, the biosurfactant at the liquid-gas interface does not bear a charge. However, the stimulus alters the surface charge of the liquid-gas interface and the hydration structure of the biosurfactant at the liquid-gas interface. In some embodiments, the stimulus is a kosmotropic salt or chaotropic salt.

In some embodiments, the stimulus stabilizes or maintains the foam. In some embodiments, the stimulus destabilizes the foam and/or causes it to collapse.

In some embodiments, the method further comprises the step of:
ii) exposing the biosurfactant to a second stimulus that alters the zeta potential and/or surface charge of the biosurfactant at the liquid-gas interface or the metal ion binding of the biosurfactant or hydration structure of the biosurfactant at the liquid-gas interface adopted on exposure to the stimulus in step i).

In some embodiments, steps i) and/or ii) are repeated one or more times.

In some embodiments, the foam further comprises an α-helical peptide, an antimicrobial peptide and/or an enzyme selected from a protease, amylase, lipase or cellulase.

In a particular embodiment, there is provided a method of modulating the stability of a foam comprising the steps of:
i) forming a stable foam from a foaming composition; said foaming composition comprising:
   a. a first bulk aqueous phase having a pH of 8 or above;
   b. a biosurfactant having at least two α-helical peptides linked by a linking sequence of 3 to 11 amino acid residues, each α-helical peptide comprises a sequence of amino acid residues:
      (a b c d d′ e f g)ₙ
   wherein n is an integer from 2 to 12;
   amino acid residues a and d are hydrophobic amino acid residues;
   amino acid residue d′ is absent or is a hydrophobic amino acid residue;
   at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
   amino acid residue g is any amino acid residue;
   wherein each α-helical peptide comprises a lysine residue;
ii) removing a substantial fraction of the first bulk aqueous phase from the foaming composition; and
iii) replacing the first bulk aqueous phase with a second bulk aqueous phase having a pH below 8.

In some embodiments, the first bulk aqueous phase has a pH of about 8.3 to 9.0, especially 8.5 to 9.0. In some embodiments, the second bulk aqueous phase has a pH of about 7.0 to 7.7, especially 7.0 to 7.5. In some embodiments, the foaming composition further comprises an α-helical peptide, an antimicrobial peptide and/or an enzyme selected from a protease, amylase, lipase or cellulase. This embodiment is particularly useful in controlling foam stability during a laundry wash cycle and foam collapse at the beginning of a laundry rinse cycle.

In another aspect of the invention, there is provided a method of purifying a polypeptide or protein biosurfactant that has a folded tertiary structure with a substantially hydrophobic core and a substantially hydrophilic surface; said method comprising the steps of:
i) treating a composition comprising the polypeptide or protein biosurfactant and other cell based protein, polypeptide and/or peptide contaminants with a kosmotropic salt in an amount suitable to salt-out the contaminants to form a precipitate and to salt-in the polypeptide or protein in solution; and
ii) separating the precipitate from the solution containing the polypeptide or protein.

In some embodiments, step i) is performed at atmospheric or ambient pressure and a temperature above 45° C., for example 60° C. or above, especially at a temperature in the range of 85° C. to 100° C., more especially about 90° C. to 95° C. Alternatively, step i) may be performed at elevated pressure and a temperature above 100° C., for example by autoclaving. In yet other embodiments, step i) may be performed at reduced pressure and a temperature of less than 60° C. In some embodiments, the kosmotropic salt is a sulphate, especially ammonium or sodium sulphate. In some embodiments, the amount of kosmotropic salt is in the range of 0.2 M to 1.5 M. In some embodiments, the folded tertiary structure is a helix bundle, especially a four helix bundle.

In yet another aspect of the invention there is provided a method of purifying a polypeptide or protein that has a folded tertiary structure with a substantially hydrophobic core and a substantially hydrophilic surface; said method comprising the step of:
i) treating a composition comprising microorganism cells containing the polypeptide or protein with a kosmotropic salt at a temperature of at least 45° C.

In some embodiments step i) is performed at atmospheric pressure and a temperature is at least 60° C., especially in the range of 85° C. to 100° C., more especially about 90° C. to 95° C. Alternatively, step i) may be performed at elevated pressure and a temperature above 100° C., for example by autoclaving. In yet other embodiments, step i) may be performed at reduced pressure and a temperature of less than 60° C. In some embodiments the kosmotropic salt is a sulphate, especially ammonium or sodium sulphate. In some embodiments, the amount of kosmotropic salt is in the range of 0.2 M to 1.5 M. In some embodiments the folded tertiary structure is a helix bundle, especially a 4 helix bundle.

In a further aspect of the invention, there is a method of manufacturing a polypeptide or protein that has a folded tertiary structure with a substantially hydrophobic core and a substantially hydrophilic surface; said method comprising:
i) providing a microorganism containing a polynucleotide sequence, wherein the polynucleotide sequence comprises a nucleotide sequence that encodes the polypeptide or protein, and wherein the nucleotide sequence is operably linked to a promoter sequence;
ii) culturing the microorganism to express the polypeptide or protein;
iii) disrupting the microorganism cells to form a cell disruptate composition;
iv) treating the disruptate with a kosmotropic salt in an amount suitable to salt-out cell based protein, polypeptide and peptide contaminants to form a precipitate and salt-in the polypeptide or protein in solution; and
v) separating the precipitate from the solution of polypeptide or protein.

In some embodiments, step iv) is performed at atmospheric pressure and a temperature of at least 45° C., for example 60° C. or above, especially a temperature in the range of 85° C. to 100° C., especially about 90° C. to 95° C. Alternatively, step iv) may be performed at elevated pressure and a temperature above 100° C., for example by autoclaving. In yet other embodiments, step iv) may be performed at reduced pressure and a temperature of less than 60° C.

In some embodiments, steps iii) and iv) are performed concurrently at atmospheric pressure and a temperature of at least 45° C., for example 60° C. or above, especially a temperature in the range of 85° C. to 100° C., especially about 90° C. to 95° C. Alternatively, steps iii) and iv) may be performed at elevated pressure and a temperature above 100° C., for example by autoclaving. In yet other embodiments, steps iii) and iv) may be performed at reduced pressure and a temperature of less than 60° C. In some embodiments, step iii) and/or step iv) is performed at acidic pH, especially a pH less than 6, more especially less than 5, for example between pH 3 and 4.5, especially about pH 4. In some embodiments, the kosmotropic salt is a sulphate, especially ammonium or sodium sulphate. In some embodiments, the amount of kosmotropic salt is in the range of 0.2 M to 1.5 M. In some embodiments, the folded tertiary structure is a helix bundle, especially a four helix bundle.

In some embodiments of this method, the polypeptide or protein comprises at least two a-helical peptides linked by a linking sequence of 3 to 11 amino acid residues, wherein each a-helical peptide comprises a sequence of amino acid residues:
(a b c d d′ e f g)ₙ
   wherein n is an integer from 2 to 12;
   amino acid residues a and d are hydrophobic amino acid residues;
   amino acid residue d′ is absent or is a hydrophobic amino acid residue;
   at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
   amino acid residue g is any amino acid residue.

In some embodiments, the linking sequence comprises a cleavable bond and the method further comprises the step of cleaving the cleavable bond. In some embodiments, each sequence (a b c d d′ e f g) in the α-helical peptide comprises at least one glutamine or asparagine residue and no net charge or each sequence (a b c d d′ e f g) comprises at least one glutamine or asparagine residue and one negative charge and the method further comprises the step of deamidating the glutamine and/or asparagine residues.

In some embodiments, the polynucleotide sequence further encodes a second protein, polypeptide or peptide and a cleavable linker operably linked with the nucleotide sequence encoding the polypeptide or protein, such that step ii) expresses a fusion protein comprising the second protein, polypeptide or peptide cleavably linked to the polypeptide or protein. In some embodiments, the method further comprises the step of cleaving the cleavable linker of the fusion protein. In some embodiments, the second protein, polypeptide or peptide is an antimicrobial peptide.

In yet another aspect of the invention there is provided a composition comprising a polypeptide or protein of the invention and an α-helical peptide of the invention.

In some embodiments, the polypeptide or protein is a polypeptide or protein of SEQ ID NO:1. In some embodiments, the α-helical peptide is a peptide of SEQ ID NO:12. In particular embodiments, the polypeptide or protein is a polypeptide or protein of SEQ ID NO:1 and the α-helical peptide is a peptide of SEQ ID NO:12.

### BRIEF DESCRIPTION OF THE FIGURES

FIG. 1. SDS-PAGE gel showing the results of ammonium sulphate (AS) precipitation in cell disruptates comprising SEQ ID NO:1 under different conditions. All experiments performed at OD₆₀₀ of 15. Lane 1: pH 7, 1.5 M AS, supernatant; Lane 2: pH 7, 1.5 M AS, pellet; Lane 3: pH 4, 1.5 M AS, supernatant; Lane 4: pH 4, 1.5 M AS, pellet; Lane 5: pH 3, 1.5 M AS, supernatant; Lane 6: pH 3, 1.5 M AS, pellet; Lane 7: pH 4, 1.0 M, supernatant; Lane 8: pH 4, 1.0 M AS, pellet; Lane 9: pH 4, 0.5 M AS, supernatant; Lane 10: pH 4, 0.5 M AS, pellet; Lane 11: pH 4, 0 M AS supernatant; Lane 12: pH 4, 0 M AS, pellet; Lane 13: -; Lane 14: clarified disruptate (ref.); Lane 15: protein marker.

FIG. 2. SDS-PAGE gel showing SEQ ID NO:1 after treatment with AS and heat. Lane 1: protein marker; Lane 2: Fermentation broth before induction of protein expression; Lane 3: Fermentation broth after induction of protein expression; Lane 4: Sample 1 after AS addition and heat treatment; Lane 5: Supernatant of treated Sample 1; Lane 6: Pellet of treated Sample 1; Lane 7: Sample 2 after AS addition and heat treatment; Lane 8: Supernatant of treated Sample 2; Lane 9: Pellet of treated Sample 2; Lane 10: Sample 3 after AS addition and heat treatment; Lane 11: Supernatant of treated Sample 3; Lane 12: Pellet of treated Sample 3.

FIG. 3. SDS-PAGE gel showing SEQ ID NO:7 after treatment with AS and heat in deionized water (A) and culture medium (B). Lane 1: Soluble protein fraction of sonicated cells; Lane 2: Insoluble protein fraction of sonicated cells; Lane 3: Supernatant of Sample 1 after heat treatment of cell suspension; Lane 4: Pellet of Sample 1 after heat treatment of cell suspension; Lane 5: Supernatant of Sample 2 after 0.5 M AS addition and heat treatment of cell suspension; Lane 6: Pellet of Sample 2 after 0.5 M AS addition and heat treatment of cell suspension; Lane 7: Supernatant of Sample 3 after 1.5 M AS addition and heat treatment of cell suspension; Lane 8: Pellet of Sample 3 after 1.5 M AS addition and heat treatment of cell suspension; Lane 9: Supernatant of Sample 4 after 1.5 M AS addition and heat treatment of sonicated cells; Lane 10: Pellet of Sample 4 after 1.5 M AS addition and heat treatment of sonicated cells; Lane 11: Protein marker.

FIG. 4. Block flow diagram of the manufacturing process without heat treatment as applied to SEQ ID NO:1.

FIG. 5. Block flow diagram of the manufacturing process with heat treatment as applied to SEQ ID NO:1.

FIG. 6. Metal-ion dependent switch. 0.3 mg/mL SEQ ID NO:1, 10 mM NaCl, 25 mM HEPES pH 7.4, 500 μM Zn. a) 20 μL of 100 mM EDTA added to top of foam, pump kept running. b) Control: no addition, pump kept running.

FIG. 7. pH switch with acid. 0.3 mg/mL SEQ ID NO:1, 10 mM NaCl, 25 mM HEPES pH 8.5, 200 μM EDTA. a) 14 μL 1 M HCl added to top of foam, bulk pH 7.5 after foam collapse. b) 14 μL 1 M NaCl added to top of foam, no pH change. c) 7 μL 1 M H₂SO₄ added to top of foam, bulk pH 7.5 after foam collapse.

FIG. 8. Foam control by pH dilution. 0.3 mg/mL SEQ ID NO:1 in Milli-Q with 200 μM EDTA. a) pH 8.5, sample shaken for 30 sec then left to stand for 15 min. b) the drained liquid from (a) (85% original volume) removed and replaced with Milli-Q water, then the sample shaken for 30 sec. Bulk pH dropped to 7.1.

FIG. 9. Water structuring switch. 0.3 mg/mL SEQ ID NO:1, 10 mM NaCl, 25 mM HEPES pH 8.5, 200 μM EDTA. a) 200 μL 3 M NaSCN added to top of foam (to give 500 mM bulk NaSCN concentration). b) 1 mL 1 M Na₂SO₄ added to top of foam (to give 500 mM bulk Na₂SO₄ concentration).

FIG. 10. Circular dichroism spectra showing α-helical structure of SEQ ID NO:1. Sample: 0.025 mg/mL SEQ ID NO:1, 200 μM EDTA, black line: pH 8.5, grey line: pH 7.5. Solid lines: 25° C., dotted lines: 90° C.

FIG. 11. SDS-PAGE gel showing expression of SEQ ID NOs: 8, 10 and 11. Lane 1: Protein marker; Lane 2: SEQ ID NO:8, 0 hr, total protein; Lane 3: SEQ ID NO:8, 0 hr, soluble protein; Lane 4: SEQ ID NO:8, overnight, total protein; Lane 5: SEQ ID NO:8, overnight, soluble protein; Lane 6: SEQ ID NO:10, 0 hr, total protein; Lane 7: SEQ ID NO:10, 0 hr, soluble protein; Lane 8: SEQ ID NO:10, overnight, total protein; Lane 9: SEQ ID NO:10, overnight, soluble protein; Lane 10: SEQ ID NO:11, 0 hr, total protein; Lane 11: SEQ ID NO:11, 0 hr, soluble protein; Lane 12: SEQ ID NO:11, overnight, total protein; Lane 13: SEQ ID NO:11, overnight, soluble protein;

FIG. 12. SDS-PAGE gel showing purification of SEQ ID NO:1 in a single step thermal treatment with and without AS. Lane 1: Sample 1, Supernatant, 1.5 M AS and heat treatment; Lane 2: Sample 2, Supernatant, 1.5 M AS and heat treatment; Lane 3: Sample 3, Supernatant, heat treatment only; Lane 4: Sample 4, Supernatant, heat treatment only; Lane 5: Protein marker.

FIG. 13. SDS-PAGE gel showing SEQ ID NO:11 after treatment of prior non-broken cells with AS and heat in Milli-Q water. SEQ ID NO:11 contains no basic groups and therefore does not bind the dye used and shows up a negatively stained. Lane 1: Protein marker; Lane 2: Total protein of Sample 1 after heat treatment; Lane 3: Supernatant of Sample 1 after heat treatment; Lane 4: Pellet of Sample 1 after heat treatment; Lane 5: Total protein of Sample 2 after 0.5 M AS addition and heat treatment; Lane 6: Supernatant of Sample 2 after 0.5 M AS addition and heat treatment; Lane 7: Pellet of Sample 2 after 0.5 M AS addition and heat treatment; Lane 8: Total protein of Sample 3 after 1 M AS addition and heat treatment; Lane 9: Supernatant of Sample 3 after 1 M AS addition and heat treatment; Lane 10: Pellet of Sample 3 after 1 M AS addition and heat treatment; Lane 11: Total protein of Sample 4 after 1.5 M AS addition and heat treatment; Lane 12: Supernatant of Sample 4 after 1.5 M AS addition and heat treatment; Lane 13: Pellet of Sample 4 after 1.5 M AS addition and heat treatment.

FIG. 14. SDS-PAGE gel showing SEQ ID NO:7 after thermal treatment in the presence of 1.0 M AS under varying heating conditions. A: Lane 1: Soluble protein fraction after sonication; Lane 2: Insoluble protein fraction after sonication; Lane 3: Supernatant of Sample 1 after heat treatment (90° C., 10 min); Lane 4: Pellet of Sample 1 after heat treatment (90° C., 10 min); Lane 5: Supernatant of Sample 2 after heat treatment (90° C., 30 min); Lane 6: Pellet of Sample 2 after heat treatment (90° C., 30 min); Lane 7: Supernatant of Sample 3 after heat treatment (90° C., 60 min); Lane 8: Pellet of Sample 3 after heat treatment (90° C., 60 min); Lane 9: Protein marker. B: Lane 1: Soluble protein fraction after sonication; Lane 2: Insoluble protein fraction after sonication; Lane 3: Supernatant of Sample 4 after heat treatment (80° C., 20 min); Lane 4: Pellet of Sample 4 after heat treatment (80° C., 20 min); Lane 5: Supernatant of Sample 5 after heat treatment (90° C., 20 min); Lane 6: Pellet of Sample 5 after heat treatment (90° C., 20 min); Lane 7: Supernatant of Sample 6 after heat treatment (100° C., 20 min); Lane 8: Pellet of Sample 6 after heat treatment (100° C., 20 min); Lane 9: Protein marker.

FIG. 15. SDS-PAGE gel showing SEQ ID NO:1 after thermal treatment in the presence of AS or sodium sulphate (SS). A: Lane 1: Soluble protein fraction after sonication; Lane 2: Insoluble protein fraction after sonication; Lane 3: 0 M AS Supernatant (SN); Lane 4: 0 M AS Pellet (P); Lane 5: 0.25 M AS SN; Lane 6: 0.25 M AS P; Lane 7: 0.5 M AS SN; Lane 8: 0.5 M AS P; Lane 9: Protein marker. B: Lane 1: Soluble protein fraction after sonication; Lane 2: Insoluble protein fraction after sonication; Lane 3: 1.5 M AS SN; Lane 4: 1.5 M AS P; Lane 5: 0.5M SS SN; Lane 6: 0.5M SS P; Lane 7: 1.0M SS SN; Lane 8: 1.0 M SS P; Lane 9: Protein marker.

FIG. 16. SDS-PAGE gel showing SEQ ID NO:7 after thermal treatment in the presence of different salts, ammonium sulphate (AS), sodium sulphate (SS) and calcium chloride (CC). A: Lane 1: Soluble protein fraction after sonication; Lane 2: Insoluble protein fraction after sonication; Lane 3: Supernatant of Sample 1 (0 M AS) after heat treatment; Lane 4: Pellet of Sample 1 (0 M AS) after heat treatment; Lane 5: Supernatant of Sample 2 (0.25 M AS) after heat treatment; Lane 6: Pellet of Sample 2 (0.25 M AS) after heat treatment; Lane 7: Supernatant of Sample 3 (0.5 M AS) after heat treatment; Lane 8: Pellet of Sample 3 (0.5 M AS) after heat treatment; Lane 9: Protein marker. B: Lane 1: Supernatant of Sample 4 (0.25 M SS) after heat treatment; Lane 2: Pellet of Sample 4 (0.25 M SS) after heat treatment; Lane 3: Supernatant of Sample 5 (0.5 M SS) after heat treatment; Lane 4: -(pellet not recovered); Lane 5: Supernatant of Sample 6 (0.25 M CC) after heat treatment; Lane 6: pellet of Sample 6 (0.25 M CC) after heat treatment; Lane 7: Supernatant of Sample 7 (0.5 M CC) after heat treatment; Lane 8: Pellet of Sample 7 (0.5 M CC) after heat treatment; Lane 9: Protein marker.

FIG. 17. Conceptual depiction of SEQ ID NO:1 and its theoretical cleavage into four a-helical peptides of SEQ ID NO:12.

FIG. 18. A) HPLC trace of SEQ ID NO:1 cleavage kinetics. At 0 hours, only SEQ ID NO:1 is present. At 6 hours, three distinct extra regions of peaks are visible, which eventually disappear into only one region (15 and 30 hours). B) HPLC trace of heat treating synthetic SEQ ID NO:12. A range of peaks is obtained, as seen when heat/acid cleaving SEQ ID NO:1 (A).

FIG. 19. Photographic depiction of foam stability for SEQ ID NO:1, SEQ ID NO:12 and a mixture thereof. All foams are in 25 mM HEPES, 200 μM EDTA at pH 8.5. Left images are the foams formed after 10 minutes of bubbling air at 1 mL min⁻¹, and right images show foam stability after 1 hour of standing. A) 0.3 mg/mL SEQ ID NO:12, forms a very fine foam which is very stable even after 1 hour. B) 0.3 mg/mL SEQ ID NO:1, forms a foam of comparable height to SEQ ID NO:12 but coarser bubbles, and has inferior 1 hour stability. C) 0.15 mg/mL SEQ ID NO:1+0.15 mg/mL SEQ ID NO:12, combining the two components yields a fine foam with better stability that SEQ ID NO:1 alone.

FIG. 20. Photographic depiction of foam stability of SEQ ID NO:1 with varying concentrations of SEQ ID NO:12, all samples in 10 mM NaCl, 200 μM EDTA, 25 mM HEPES, pH 8.5 bubbled for 10 minutes with air and observed over 1 hour. A. 0.3 mg/mL SEQ ID NO:1 forms a substantial foam after 10 minutes bubbling which decreases to less than half its original height after 1 hour standing. B. 0.03 mg/mL SEQ ID NO:12/0.27 mg/mL SEQ ID NO:1 forms a foam of higher quality with smaller bubble size and foam stability increases. C. 0.1 mg/mL SEQ ID NO:12/0.2 mg/mL SEQ ID NO:1 further increases in foam quality and stability are observed. D. 0.2 mg/mL SEQ ID NO:12/0.1 mg/mL SEQ ID NO:1 provides a further increase in foam quality and stability.

FIG. 21. Photographic depiction of foam switching by addition of acid. A substantial high quality foam was formed by bubbling air through 0.15 mg/mL SEQ ID NO:1/0.15 mg/mL SEQ ID NO:12 10 mM NaCl, 200 μM EDTA, 25 mM HEPES, pH 8.5 (left image). Addition of 14 μL 1 M HCl to neutralize the bulk solution to pH 7.4, while keeping constant air flow, dissipated the foam to a fraction of its original height in less than 2 minutes (right image).

### DETAILED DESCRIPTION OF THE INVENTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 20%, or 10% to a reference quantity, level, value, dimension, size, or amount.

As used herein the term “acid” refers to a substance that can donate one or more hydrogen ions (H⁺) to a second substance, where the receiving substance is a base. The addition of acid lowers the pH of an aqueous solution. Examples of suitable acids include inorganic acids and organic acids. Examples of suitable inorganic acids include, but are not limited to, hydrochloric acid, hydrofluoric acid, hydrobromic acid, hydroiodic acid, nitric acid, sulphuric acid and phosphoric acid. Examples of suitable organic acids include, but are not limited to, acetic acid, formic acid, propionic acid, butyric acid, benzoic acid, citric acid, tartaric acid, malic acid, maleic acid, hydroxymaleic acid, fumaric acid, lactic acid, mucic acid, gluconic acid, oxalic acid, phenylacetic acid, methanesulphonic acid, toluenesulphonic acid, benzenesulphonic acid, salicylic acid, sulphanilic acid, ascorbic acid, valeric acid, succinic acid, glutaric acid and adipic acid.

As used herein the term “affinity for the liquid-gas interface” means that biosurfactant polypeptides or proteins from a bulk solution are attracted to the liquid-gas interface such that there is a positive surface excess. In general, the biosurfactant molecules have hydrophobic regions and re-organize themselves at the interface to minimize their free energy on adsorption, typically such that their hydrophobic regions are in contact with a non-polar portion of the interface and their hydrophilic regions are in contact with a polar portion of the interface.

The term “amphiphilic” refers to molecules having both hydrophilic and hydrophobic regions. The term amphiphilic is synonymous with “amphipathic” and these terms may be used interchangeably.

The term “base” as used herein refers to a substance that is capable of accepting a hydrogen ion (H⁺). The addition of base increases the pH of an aqueous solution. Examples of suitable bases include ammonia, organic amines, sodium hydroxide, potassium hydroxide, calcium hydroxide, magnesium hydroxide, sodium carbonate, potassium carbonate, magnesium carbonate, calcium carbonate, sodium bicarbonate, potassium bicarbonate, magnesium bicarbonate and calcium bicarbonate.

As used herein, the term “chaotropic salt” refers to a substance which destabilizes molecular structure, for example, by weakening or disrupting intermolecular or intramolecular interactions, hydrogen bonding or hydrophobic interactions. Examples of suitable chaotropic salts include guanidinium salts, thiocyanate salts, perchlorate salts and iodide salts, such as sodium thiocyanate, potassium iodide, guanidinium chloride and guanidinium thiocyanate.

The term “chelating agent” as used herein refers to a compound that can form a complex with a metal ion. In particular, chelating agents are bi- or polydentate metal ion ligands having at least two heteroatoms capable of simultaneously coordinating with the metal ion. Illustrative examples of chelating agents suitable for use in the invention include ethylenediamine, ethylenetriamine, triethylenetetramine, ethylenediaminetetraacetic acid (EDTA), aminoethanolamine, ethylene glycol bis(2-aminoethyl ether)-N,N,N′N′-tetraacetic acid (EGTA), tris(2-imidazolyl)carbinol, tris[4(5)-imidazolyl]carbinol, bis[4(5)-imidazolyl]glycolic acid, oxaloacetic acid, citric acid, glycine or other amino acids, salicylate, macrocyclic ethers, multidentate Schiff bases, acetylacetone, bis(acetylacetone) ethylenediimine, 2-nitroso-1-naphthol, 3-methoxyl-2-nitrosophenol, cyclohexanetrione trioxime, diethylenetriaminepentaacetic acid (DTPA), N-(hydroxyethyl)ethylenediaminetriacetic acid (HEDTA), tripolyphosphate ion, nitrilotriacetic acid, dimethylglyoxime, dimercaprol and deferoxamine.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the term “deamidating” and variations such as “deamidation” or “deamidate” refer to the hydrolysis of the amine group from an amide to form a carboxylic acid.

As used herein, the term “foam” refers to a dispersion of gas bubbles in or on a liquid. The gas bubbles may be dispersed throughout the liquid phase in a heterogeneous or homogeneous manner. Illustrative examples of foams include gases such as air, nitrogen, oxygen, helium or hydrogen entrapped in a liquid such as water or an oil. A foam may be transient, unstable or stable.

The term “helix bundle” refers to a series of peptide helices that fold such that the helices are substantially parallel or anti-parallel to one another. A two helix bundle has two helices folded such that they are substantially parallel or anti-parallel to one another. A three helix bundle has three helices folded such that they are substantially parallel or anti-parallel to one another. A four helix bundle has four helices folded such that they are substantially parallel or anti-parallel to one another. A five helix bundle has five helices folded such that they are substantially parallel or anti-parallel to one another. By “substantially parallel or anti-parallel” it is meant that the helices are folded such that the side chains of the helices are able to interact with one another. For example, the hydrophobic side chains of the helices are able to interact with one another to form a hydrophobic core.

The term “hydrophilic” refers to a molecule or portion of a molecule that is attracted to water and other polar solvents. A hydrophilic molecule or portion of a molecule is polar and/or charged or has an ability to form interactions such as hydrogen bonds with water or polar solvents.

As used herein the term “substantially hydrophilic surface” as used herein refers to the outer surface of the tertiary structure of a protein or polypeptide that is in contact with the liquid phase and is predominantly hydrophilic. The hydrophilic surface presents polar and/or charged amino acid side chains on the outer surface which are in contact with the liquid phase. While non-polar amino acid side chains may be present on the hydrophilic surface, they do not appear spatially adjacent to one another in the tertiary structure so as to form a significant hydrophobic area within the hydrophilic surface.

The term “hydrophobic” refers to a molecule or portion of a molecule that repels or is repelled by water and other polar solvents. A hydrophobic molecule or portion of a molecule is non-polar, does not bear a charge and is attracted to non-polar solvents.

As used herein, the term “substantially hydrophobic core” refers to the internal portion of the tertiary structure of a protein or polypeptide that is not in contact with the liquid phase and is predominantly hydrophobic. The amino acid side chains in the hydrophobic core are predominantly non-polar. While polar amino acid side chains may be present in or close to the hydrophobic core, their number is insufficient to disrupt the folding of the protein or polypeptide.

As used herein, the terms “interact”, “interacts”, “interaction” and “interacting” refer to attractive forces that occur within a polypeptide or protein or between polypeptide or protein molecules. The attractive forces may be responsible for the conformation adopted by a polypeptide or protein and thereby influence the affinity of the polypeptide or protein for the liquid-gas interface, or may promote or discourage association with other polypeptides or proteins. The attractive forces may also be intermolecular thereby encouraging the polypeptides or proteins located at the liquid-gas interface to associate with one another or with polypeptides or proteins adsorbed at a second interface within the two interface structure of a foam thin film. Illustrative examples of suitable interactions include ion-pair interactions, dipole interactions, London dispersion forces, salt bridge formation, hydrogen bonding and short range solvation forces such as hydration, hydrophobic interactions, osmotic attractive potential due to the exclusion of ions, and surface charge interactions. In some cases, intermolecular or intramolecular covalent bonding, such as disulfide bond formation may occur between polypeptide or protein molecules. In the context of modulation of foam stability, covalent bonding between polypeptide or protein biosurfactants at a liquid-gas interface is less desirable if stimuli-responsive collapse of the foam is desired.

The term “kosmotropic salt” refers to a substance which stabilizes molecular structure, for example, by strengthening intermolecular or intramolecular interactions or by introducing new structure to the hydration layer in the proximity of the polypeptide or protein. Examples of kosmotropic salts include sulphates, fluorides, carbonates, magnesium salts, lithium salts and calcium salts, including ammonium sulphate, sodium sulphate, calcium chloride and lithium chloride.

As used herein, the term “liquid-gas interface” refers to a surface forming the common boundary between a gas phase and a liquid phase, for example, air and water or air and oil.

As used herein, the terms “modulate”, “modulation” and “modulating” refer to a regulation or adjustment to a certain measure or proportion. Modulation when applied to stabilization of a foam refers to enhancement, reduction or abolition of stability. For example, modulation when applied to foam stability refers to a stabilization of the foam, a destabilization of the foam or collapse of the foam.

As used herein, the terms “peptide”, “polypeptide” and “protein” refer to two or more naturally occurring or non-naturally occurring amino acids joined by peptide bonds. While there are no rules that govern the boundaries between these terms, generally peptides contain less amino acid residues than polypeptides and polypeptides contain less amino acid residues than proteins.

As used herein, the term “amino acid” refers to an α-amino acid or a β-amino acid and may be a L- or D-isomer. The amino acid may have a naturally occurring side chain (see Table 1) or a non-naturally occurring side chain (see Table 2). The amino acid may also be further substituted in the a-position or the β-position with a group selected from —C₁-C₆alkyl, —(CH₂)₆COR₁, —(CH₂)₆R₂, —PO₃H, —(CH₂)ₙheterocyclyl or —(CH₂)ₙaryl where R₁ is —OH, —NH₂, —NHC₁-C₃alkyl, —OC₁-C₃alkyl or —C₁-C₃alkyl and R₂ is —OH, —SH, —SC₁-C₃alkyl, —OC₁-C₃alkyl, —C₃-C₁₂cycloalkyl, —NH₂, —NHC₁-C₃alkyl or —NHC(C═NH)NH₂ and where each alkyl, cycloalkyl, aryl or heterocyclyl group may be substituted with one or more groups selected from —OH, —NH₂, —NHC₁-C₃alkyl, —OC₁-C₃alkyl, —SH, —SC₁-C₃alkyl, —CO₂H, —CO₂C₁-C₃alkyl, —CONH₂ or —CONHC₁-C₃alkyl.

Amino acid structure and single and three letter abbreviations used throughout the specification are defined in Table 1, which lists the twenty naturally occurring amino acids which occur in proteins as L-isomers.

TABLE 1

| | |
|---|---|
| | (1) |
| 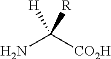 | |
| | (2) |
| 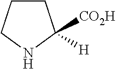 | |

| Amino | Three-letter | One-letter | Structure of side |
|---|---|---|---|
| Acid | Abbreviation | symbol | chain (R) |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indolyl) |
| Tyrosine | Tyr | Y | —CH₂(4-hydroxyphenyl) |
| Valine | Val | V | —CH(CH₃)₂ |

The term “α-amino acid” as used herein, refers to a compound having an amino group and a carboxyl group in which the amino group and the carboxyl group are separated by a single carbon atom, the α-carbon atom. An α-amino acid includes naturally occurring and non-naturally occurring L-amino acids and their D-isomers and derivatives thereof such as salts or derivatives where functional groups are protected by suitable protecting groups. The α-amino acid may also be further substituted in the a-position with a group selected from —C₁-C₆alkyl, —(CH₂)ₙCOR₁, —(CH₂)ₙR₂, —PO₃H, —(CH₂)ₙheterocyclyl or —(CH₂)ₙaryl where R₁ is —OH, —NH₂, —NHC₁-C₃alkyl, —OC₁-C₃alkyl or —C₁-C₃alkyl and R₂ is —OH, —SH, —SC₁-C₃alkyl, —OC₁-C₃alkyl, —C₃-C₁₂cycloalkyl, —NH₂, —NHC₁-C₃alkyl or —NHC(C═NH)NH₂ and where each alkyl, cycloalkyl, aryl or heterocyclyl group may be substituted with one or more groups selected from —OH, —NH₂, —NHC₁-C₃alkyl, —OC₁-C₃alkyl, —SH, —SC₁-C₃alkyl, —CO₂H, —CO₂C₁-C₃alkyl, —CONH₂ or —CONHC₁-C₃alkyl.

As used herein, the term “β-amino acid” refers to an amino acid that differs from an a-amino acid in that there are two (2) carbon atoms separating the carboxyl terminus and the amino terminus. As such, β-amino acids with a specific side chain can exist as the R or S enantiomers at either of the a (C2) carbon or the β (C3) carbon, resulting in a total of 4 possible isomers for any given side chain. The side chains may be the same as those of naturally occurring α-amino acids (see Table 1 above) or may be the side chains of non-naturally occurring amino acids (see Table 2 below).

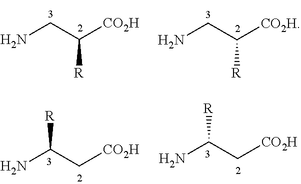

Furthermore, the α-amino acids may have mono-, di-, tri- or tetra-substitution at the C2 and C3 carbon atoms. Mono-substitution may be at the C2 or C3 carbon atom. Di-substitution includes two substituents at the C2 carbon atom, two substituents at the C3 carbon atom or one substituent at each of the C2 and C3 carbon atoms. Tri-substitution includes two substituents at the C2 carbon atom and one substituent at the C3 carbon atom or two substituents at the C3 carbon atom and one substituent at the C2 carbon atom. Tetra-substitution provides for two substituents at the C2 carbon atom and two substituents at the C3 carbon atom. Suitable substituents include —C₁-C₆alkyl, —(CH₂)ₙCOR₁, —(CH₂)ₙR₂, —PO₃H, —(CH₂)ₙheterocyclyl or —(CH₂)ₙaryl where R₁ is —OH, —NH₂, —NHC₁-C₃alkyl, —OC₁-C₃alkyl or —C₁-C₃alkyl and R₂ is —OH, —SH, —SC₁-C₃alkyl, —OC₁-C₃alkyl, —C₃-C₁₂cycloalkyl, —NH₂, —NHC₁-C₃alkyl or —NHC(C═NH)NH₂ and where each alkyl, cycloalkyl, aryl or heterocyclyl group may be substituted with one or more groups selected from —OH, —NH₂, —NHC₁-C₃alkyl, —OC₁-C₃alkyl, —SH, —SC₁-C₃alkyl, —CO₂H, —CO₂C₁-C₃alkyl, —CONH₂ or —CONHC₁-C₃alkyl.

Other suitable β-amino acids include conformationally constrained β-amino acids. Cyclic β-amino acids are conformationally constrained and are generally not accessible to enzymatic degradation. Suitable cyclic β-amino acids include, but are not limited to, cis- and trans-2-aminocyclopropyl carboxylic acids, 2-aminocyclobutyl and cyclobutenyl carboxylic acids, 2-aminocyclopentyl and cyclopentenyl carboxylic acids, 2-aminocyclohexyl and cyclohexenyl carboxylic acids and 2-amino-norbornane carboxylic acids and their derivatives, some of which are shown below:

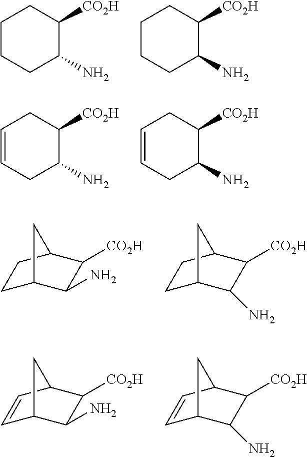

Suitable derivatives of β-amino acids include salts and may have functional groups protected by suitable protecting groups.

The term “non-naturally occurring amino acid” as used herein, refers to amino acids having a side chain that does not occur in the naturally occurring L-a-amino acids. Examples of non-natural amino acids and derivatives include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, citrulline, sarcosine, 4-amino-3-hydroxy-6-methylheptanoic acid, 2-thienyl alanine and/or D-isomers of amino acids. A list of unnatural amino acids that may be useful herein is shown in Table 2.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisoleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylasparatate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylorinithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methylnapthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylasparate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2- | Nmbc | | |
| diphenylethylamino)cyclopropane | | | |

The term “alkyl” as used herein refers to straight chain or branched hydrocarbon groups. Suitable alkyl groups include, but are not limited to methyl, ethyl, propyl, isopropyl, butyl, isobutyl, pentyl, hexyl, heptyl, octyl, nonyl, decyl, dodecyl, tetradecyl, hexadecyl and octadecyl. The term alkyl may be prefixed by a specified number of carbon atoms to indicate the number of carbon atoms or a range of numbers of carbon atoms that may be present in the alkyl group. For example; C₁-C₃alkyl refers to methyl, ethyl, propyl and isopropyl.

The term “alkenyl” as used herein refers to straight chain or branched hydrocarbon groups containing at least one double bond. Suitable alkenyl groups include, but are not limited to vinyl, propenyl, 1-butenyl, 2-butenyl, 3-butenyl, 3-methyl-2-butenyl, 1-pentenyl, 2-pentenyl, 3-pentenyl, 4-pentenyl, 3-methyl-2-pentenyl, 4-methyl-3-pentenyl, 2,4-pentadiene, 1-hexenyl, 2-hexenyl, 3-hexenyl, 4-hexenyl, 5-hexenyl, 3-methyl-2-hexenyl, 4-methyl-3-hexenyl and 5-methyl-4-hexenyl.

The term “alkynyl” as used herein refers to straight chain or branched hydrocarbon groups containing at least one triple bond. Suitable alkynyl groups include, but are not limited to ethynyl, 2-propynyl, 2-butynyl, 3-butynyl, 2-pentynyl, 3-pentynyl, 4-pentynyl, 2-hexynyl, 3-hexynyl, 4-hexynyl and 5-hexynyl.

The term “cycloalkyl” as used herein, refers to cyclic hydrocarbon groups. Suitable cycloalkyl groups include, but are not limited to, cyclopropyl, cyclobutyl, cyclopentyl, cyclohexyl, cycloheptyl, cyclooctyl, cyclononyl, cyclodecyl, cycloundecyl and cyclododecyl.

The term “heterocyclyl” as used herein refers to 5 or 6 membered saturated, partially unsaturated or aromatic cyclic hydrocarbon groups in which at least one carbon atom has been replaced by N, O or S. Optionally, the heterocyclyl group may be fused to a phenyl ring. Suitable heterocyclyl groups include, but are not limited to pyrrolidinyl, piperidinyl, pyrrolyl, thiophenyl, furanyl, oxazolyl, imidazolyl, thiazolyl, isoxazolyl, pyrazolyl, isothiazolyl, pyridinyl, quinolinyl, isoquinolinyl, indolyl, benzofuranyl, benzothiophenyl, oxadiazolyl, tetrazolyl, triazolyl and pyrimidinyl.

The term “aryl” as used herein, refers to C₆-C₁₀ aromatic hydrocarbon groups, for example phenyl and naphthyl.

The term “α-helix breaking amino acid residue” refers to an amino acid residue that has a low frequency of occurrence in natural α-helical conformations and which promotes termination of an α-helix. α-Helix breaking amino acid residues may lack an amide hydrogen to participate in hydrogen bonding within the helix or may be too conformationally flexible or inflexible to form the constrained α-helical conformation in an energy efficient manner. Examples of α-helix breaking amino acid residues include, but are not limited to praline and glycine.

The term “hydrophilic amino acid residue” as used herein refers to an amino acid residue in which the side chain is polar or charged. Examples include glycine, L-serine, L-threonine, L-cysteine, L-tyrosine, L-asparagine, L-glutamine, L-aspartic acid, L-glutamic acid, L-lysine, L-arginine, L-histidine, L-ornithine, D-serine, D-threonine, D-cysteine, D-tyrosine, D-asparagine, D-glutamine, D-aspartic acid, D-glutamic acid, D-lysine, D-arginine, D-histidine and D-ornithine, especially L-serine, L-threonine, L-cysteine, L-tyrosine, L-asparagine, L-glutamine, L-aspartic acid, L-glutamic acid, L-lysine, L-arginine, L-histidine and L-ornithine.

As used herein, the term “hydrophobic amino acid residue” refers to an amino acid residue in which the side chain is non-polar. Examples include, but are not limited to L-alanine, L-valine, L-leucine, L-isoleucine, L-proline, L-methionine, L-phenylalanine, L-tryptophan, L-aminoisobutyric acid, D-alanine, D-valine, D-leucine, D-isoleucine, D-proline, D-methionine, D-phenylalanine, D-tryptophan, D-aminoisobutyric acid, L-cyclohexylalanine, D-cyclohexylalanine, L-cyclopentylalanine, D-cyclopentylalanine, L-norleucine, D-norleucine, L-norvaline, D-norvaline, L-tert-butylglycine, D-tert-butylglycine, L-ethylglycine and D-ethylglycine, especially L-alanine, L-valine, L-leucine, L-isoleucine, L-proline, L-methionine, L-phenylalanine, L-tryptophan and L-aminoisobutyric acid.

As used herein, the term “positively charged amino acid residue” refers to an amino acid residue having a side chain capable of bearing a positive charge. Examples include, but are not limited to L-lysine, L-arginine, L-histidine, L-ornithine, D-lysine, D-arginine, D-histidine and D-ornithine.

As used herein, the term “negatively charged amino acid residue” refers to an amino acid residue having a side chain capable of bearing a negative charge. Examples include, but are not limited to L-aspartic acid, L-glutamic acid, D-aspartic acid and D-glutamic acid.

As used herein, the term “polar amino acid residue” refers to an amino acid residue having a side chain that has a dipole moment. Examples of polar amino acid residues, include, but are not limited to glycine, L-serine, L-threonine, L-cysteine, L-tyrosine, L-asparagine and L-glutamine, D-serine, D-threonine, D-cysteine, D-tyrosine, D-asparagine and D-glutamine.

The term “amino acid having a small side chain” refers to amino acid residues having a side chain with 4 or less non-hydrogen atoms, especially 3 or less non-hydrogen atoms. Examples include, but are not limited to, glycine, L-alanine, L-valine, L-leucine, L-isoleucine, L-methionine, L-serine, L-threonine, L-cysteine, L-asparagine, L-aspartic acid, D-alanine, D-valine, D-leucine, D-isoleucine, D-methionine, D-serine, D-threonine, D-cysteine, D-asparagine and D-aspartic acid, especially glycine, L-alanine, L-valine, L-serine, L-threonine and L-cysteine.

The terms “salt-in” and “salt-out” as used herein refer to a means of separating different proteins, polypeptides and peptides in a solution. Most proteins and polypeptides have a tertiary structure that has protected hydrophobic areas and unprotected hydrophilic areas. The hydrophilic areas are able to interact with surrounding solvent molecules forming hydrogen bonds. If enough of the protein or polypeptide external surface is hydrophilic, the protein or polypeptides will be soluble in water due to the extent of interactions with water. The extent of solvation of the polypeptide or protein and hence its solubility depends on the properties of the polypeptide or protein as well as the structural stability of the polypeptide or protein, for example as a function of temperature and pH, and the presence of salt ions. Salt ions may substantially modify protein solubility. While the interaction of salts with polypeptides and proteins is complex and incompletely understood, it is recognized that van der Waals interactions between salt ions and polypeptides or proteins can significantly modify the potential of mean force between polypeptides or proteins in solution and therefore their solution phase behaviour (Tavares et al., J. Phys. Chem. B, 2004, 108, 9228-9235). In the presence of increasing amounts of salt, protein-protein or polypeptide-polypeptide interactions become increasingly important and can either increase or decrease polypeptide or protein solubility. When salt increases polypeptide or protein solubility, the polypeptide or protein is said to be “salted-in”. Conversely, if the addition of salt modifies the protein-protein or polypeptide-polypeptide interactions so that the potential of mean force is attractive the protein or polypeptide molecules can associate reversibly or irreversibly and precipitate out of solution. This process is called “salting-out”. It is possible to separate proteins and polypeptides by salting-out the less soluble proteins or polypeptides while more soluble proteins or polypeptides remain in solution. Salting-out may be aided by partial or complete denaturation or unfolding of the protein or polypeptide, which has the effect of changing the chemical character and in particular the hydrophobicity of the solvent-exposed surface, in such a way that protein-protein or polypeptide-polypeptide interactions may become more attractive. Through this approach the application of heat or acid can contribute to the salting-out process. At high temperatures, for example above 60° C., most proteins denature and salting-out under these conditions typically causes irreversible precipitation in a way that is not technologically useful in protein purification.

The term “self-assembled” refers to a population of biosurfactant molecules with an affinity for the liquid-gas interface and which relocate themselves from the bulk solution to the liquid-gas interface.

As used herein, the terms “switch” and “switching” refer to turning on and/or off the stability of foam. For example, the formation or maintenance of foam during exposure to a one stimulus and/or the reduction or collapse of foam during exposure to another stimulus. Foam may be switched on and/or off multiple times.

As used herein, the term “zeta potential” refers to a measure of the magnitude of the electrostatic repulsion or attraction between surfaces. It is derived from electrophoretic mobility measurements and represents the electric potential in the interfacial double layer at the slipping plane relative the bulk fluid. A value of 25 mV (positive or negative) can be taken as the arbitrary value that separates low-charged surfaces from highly-charged surfaces. For particles that are small enough, a high zeta potential will confer stability, i.e. a foam or dispersion will resist aggregation. Foams with a high zeta potential (negative or positive) are electrically stabilized while foams with a low zeta potential tend to aggregate and/or collapse.

### Polypeptides and Proteins

The polypeptides and proteins of the present invention are polypeptides or proteins having at least 50 amino acid residues in their sequence, especially in the range of 50 to 300 amino acid residues, for example, in the range of 60 to 250 amino acid residues, 70 to 200 amino acid residues, 80 to 150 amino acid residues or 90 to 120 amino acid residues. In some embodiments, the biosurfactants have 90 to 110 amino acid residues.

In some embodiments, the polypeptide or protein has a folded tertiary structure that is a well defined bundle of α-helix subunits lacking elements of beta secondary structure. In some embodiments, the folded tertiary structure is a 2-5-helix bundle, especially a 4-helix bundle.

The polypeptides or proteins comprise at least two α-helical peptides, especially 2 to 5 α-helical peptides, more especially 2 to 4 α-helical peptides and most especially 4 α-helical peptides. Each α-helical peptide within the polypeptide or protein may be the same or different.

The α-helical peptides are linked by a sequence of 3 to 11 amino acid residues that enable folding of the α-helical peptides so that the α-helical peptides may interact with one another to form a folded tertiary structure such as a 2, 3, 4 or 5 α-helix bundle. In some embodiments, the α-helical peptides are linked by 3 to 9, 3 to 7 or 3 to 5 amino acid residues. In a particular embodiment, the α-helical peptides are linked by 3 amino acid residues.

In some embodiments, the sequence of amino acid residues linking the α-helical peptides includes an amino acid residue that is an α-helix breaking amino acid residue. This residue assists in terminating the α-helical structure of the preceding α-helical peptide and allowing the linking amino acid residues flexibility for folding. α-Helix breaking amino acid residues include amino acid residues that are unable to contribute to α-helical structure, such as proline, have high flexibility such as glycine, or have only average propensity to form α-helical structures but also confer high flexibility, for example serine. The charged group on aspartic acid is also known to have low helix propensity. Common α-helix breaking amino acid residues include proline and glycine.

The sequence of amino acid residues linking the α-helical peptides also may include one or more residues that allow flexibility so that two adjacent α-helical peptides can fold so that they interact with one another. In particular embodiments, the sequence of amino acid residues linking the α-helical peptides allows the α-helical peptides to fold in a manner to form a 2, 3, 4 or 5 helix bundle, especially a 4-helix bundle. In some embodiments, the flexibility is imparted by one or more amino acid residues having a small side chain, for example, glycine, serine, alanine, valine, cysteine and threonine. In some embodiments, these same amino acids play a dual role of conferring flexibility to the overall sequence of linking amino acid as well as helix termination.

In some embodiments, the sequence linking the α-helical peptides may contain a cleavable peptide bond. The cleavable peptide bond allows the polypeptide or protein to be cleaved into smaller peptides after or during manufacture. The cleavable peptide bond may be an acid cleavable peptide bond, a base cleavable peptide bond, a chemically cleavable peptide bond or may be a peptide bond cleavable by an enzyme such as a protease. By “acid cleavable peptide bond” is meant that the peptide bond is cleaved at a faster rate than normal peptide bonds and/or under conditions at which cleavage of other peptide bonds is negligible. Acid cleavable peptide bonds include, for example, the bond between aspartic acid and proline. Base cleavable peptide bonds include, for example, the bond between asparagine and glycine. Chemically cleavable peptide bonds include cleavage of methionine or tryptophan with cyanogen bromide and N-chlorosuccinamide respectively. Enzyme cleavable peptide bonds may be cleaved by proteases such as cysteine, serine, threonine, glutamic acid or aspartic acid proteases or metalloproteases. Examples include, but are not limited to chymotrypsin that cleaves peptide bonds following a bulky amino acid residue such as phenylalanine, tryptophan and tyrosine, trypsin that cleaves peptide bonds following a positively charged amino acid residue such as arginine, lysine or glutamine, subtilisin that has broad specificity of cleavage and elastase that cleaves peptide bonds following a small neutral amino acid residue such as alanine, glycine and valine. In some embodiments, the enzyme cleavable peptide bond is cleaved by Tobacco Etch Virus protease (TEVp), which has a very specific sequence required for cleavage thereby reducing unwanted cleavage in the polypeptide or protein. When the cleavage by TEVp is desired, the linking sequence includes the sequence E-N-L-Y-F-Q-G or E-N-L-Y-F-Q-S.

In particular embodiments, the sequence linking the α-helical peptides may comprise an acid cleavable peptide bond, especially a D-P bond.

In some embodiments, the sequence linking the α-helical peptides includes a helix-breaking amino acid residue and a cleavable peptide bond.

When more than one linking sequence is present in the polypeptide or protein, for example, where there are three to five α-helical peptides, each linking sequence may be the same or different.

In some embodiments, the linking sequence comprises D-P-X where X is a small amino acid residue such as serine, glycine, cysteine or threonine. In some embodiments, the linking sequence comprises D-P-S. In some embodiments, the linking sequence is D-P-S.

The α-helical peptide sequences in the polypeptides and proteins of the present invention comprise the sequence:
(a b c d d′ e f g).
   where n is an integer from 2 to 12, especially 2 to 6, more especially 2 to 5 and most especially 3. Each sequence (a b c d d′ e f g) in the α-helical peptide may be the same or different.

Amino acid residues a and d are hydrophobic amino acid residues. In some embodiments amino acid residues a and d are independently selected from L-alanine, L-valine, L-leucine, L-methionine, L-isoleucine, L-phenylalanine, L-tyrosine, D-alanine, D-valine, D-leucine, D-methionine, D-isoleucine, D-phenylalanine, D-tyrosine, especially L-alanine, L-methionine, L-valine and L-leucine.

Amino acid residue d′ may be absent or may be a hydrophobic amino acid residue. The residue d′ may be included in longer helix sequences, for example where n is 3, 6, 9 or 12, to counteract perturbations in the helix turn that may result in misalignment of the hydrophobic residues on one face of the helix. In some embodiments, d′ is present in the third, sixth, ninth and/or twelfth sequence of (a b c d d′ e f g)ₙ when n is 3, 6, 9 or 12, but is absent in the other (a b c d d′ e f g) sequences in an α-helical peptide. In some embodiments, when present, amino acid d′ may be selected from L-alanine, L-valine, L-leucine, L-methionine, L-isoleucine, L-phenylalanine, L-tyrosine D-alanine, D-valine, D-leucine, D-methionine, D-isoleucine, D-phenylalanine, D-tyrosine, especially L-alanine, L-methionine, L-valine and L-leucine.

At least one of residues b and c is a hydrophilic amino acid residue, such as L-serine, L-threonine, L-cysteine, L-tyrosine, L-asparagine, L-glutamine, L-aspartic acid, L-glutamic acid, L-lysine, L-histidine, L-ornithine, D-serine, D-threonine, D-cysteine, D-tyrosine, D-asparagine, D-glutamine, D-aspartic acid, D-glutamic acid, D-lysine, D-histidine and D-ornithine, especially L-serine, L-threonine, L-cysteine, L-tyrosine, L-asparagine, L-glutamine, L-aspartic acid, L-glutamic acid, L-lysine, L-histidine, L-ornithine. The other one of amino acid residues b and c is any amino acid residue, especially an amino acid residue that has a propensity to form α-helices, such as alanine, lysine, uncharged glutamic acid, methionine, leucine and aminoisobutyric acid or a small amino acid residue such as alanine, serine, valine, leucine or isoleucine, or a hydrophilic amino acid residue such as glutamine, asparagine, serine, glutamic acid and aspartic acid, provided that b and c are not both charged amino acid residues that have the same charge.

At least one of amino acid residues e and f is a hydrophilic amino acid residue, such as L-serine, L-threonine, L-cysteine, L-tyrosine, L-asparagine, L-glutamine, L-aspartic acid, L-glutamic acid, L-lysine, L-histidine, L-ornithine, D-serine, D-threonine, D-cysteine, D-tyrosine, D-asparagine, D-glutamine, D-aspartic acid, D-glutamic acid, D-lysine, D-histidine and D-ornithine, especially L-serine, L-threonine, L-cysteine, L-tyrosine, L-asparagine, L-glutamine, L-aspartic acid, L-glutamic acid, L-lysine, L-histidine and L-ornithine. The other one of amino acid residues e and f is any acid residue, especially an amino acid residue that has a propensity to form α-helices, such as alanine, lysine, uncharged glutamic acid, methionine, leucine and aminoisobutyric acid or a small amino acid residue such as alanine, valine, leucine or isoleucine or a hydrophilic amino acid residue such as glutamine, asparagine, serine, glutamic acid and aspartic acid, provided that e and f are not both charged amino acid residues that have the same charge.

Amino acid residue g may be any amino acid residue. In particular embodiments, amino acid residue g is a residue that has a propensity to form α-helices, such as alanine, lysine, uncharged glutamic acid, methionine, leucine and aminoisobutyric acid, especially alanine, lysine and uncharged glutamic acid; or amino acid residues that are not detrimental to a-helix formation, for example, amino acid residues other than proline and glycine.

In some embodiments, each amino acid residue b is independently selected from a small hydrophobic amino acid residue, such as alanine, leucine, valine and isoleucine, or a hydrophilic amino acid residue, especially a polar or positively charged amino acid residue, such as L-serine, L-threonine, L-cysteine, L-tyrosine, L-asparagine, L-glutamine, L-lysine, L-arginine or L-histidine. In some embodiments, each b is independently selected from L-lysine, L-histidine, L-serine, L-alanine, L-asparagine and L-glutamine.

In some embodiments, each amino acid residue c is independently selected from a polar, positively charged or negatively charged amino acid residue, such as L-serine, L-threonine, L-cysteine, L-tyrosine, L-asparagine, L-glutamine, L-lysine, L-arginine, L-histidine, L-aspartic acid or L-glutamic acid. In some embodiments, each c is independently selected from L-glutamine, L-arginine, L-serine, L-glutamic acid and L-asparagine.

Each amino acid residue e is independently any amino acid residue and may be hydrophobic or hydrophilic. In some embodiments, each e is independently selected from L-alanine, L-valine, L-leucine, L-isoleucine, L-serine, L-threonine, L-aspartic acid and L-glutamic acid, especially L-alanine, L-serine and L-glutamic acid.

In some embodiments, each amino acid residue f is a polar, positively charged or negatively charged amino acid residue, such as L-serine, L-threonine, L-cysteine, L-tyrosine, L-asparagine, L-glutamine, L-lysine, L-arginine, L-histidine, L-aspartic acid or L-glutamic acid. In some embodiments, each f is independently selected from L-aspartic acid, L-glutamic acid, L-arginine, L-glutamine, L-histidine, L-lysine and L-asparagine.

Amino acid residue g is independently any amino acid residue and may be hydrophobic or hydrophilic. In some embodiments, the residue g is independently selected from a small hydrophobic residue or a polar uncharged residue. In some embodiments, each g is independently selected from L-alanine, L-valine, L-leucine, L-isoleucine, L-serine, L-threonine, L-asparagine and L-glutamine, especially L-alanine, L-serine and L-glutamine.

In some embodiments, each α-helix in the polypeptide or protein comprises at least one charged amino acid residue, for example, 1 to 8 or 3 to 7 charged amino acid residues. In some embodiments, each sequence (a b c d d′ e f g) comprises at least one charged amino acid residue, especially one to three charged amino acid residues.

In some embodiments, particularly where the sequence that links the α-helical peptides include a cleavable bond such as an acid cleavable bond, the α-helical peptide sequences are designed to reduce or exclude sequences that include peptide bonds that would undergo microchemical modification such as methionine oxidation, deamidation in cases where the introduction of a charge at that position is undesirable, or would also be cleaved under the cleavage conditions.

Without wishing to be bound by theory, it is believed that in an aqueous environment the a-helical peptides within the polypeptide or protein interact with one another so that the protein or polypeptide has a substantially hydrophilic outer surface that is stabilized by an inner substantially hydrophobic core. This tertiary structure is resistant to proteases that are present in the interior of the bacterium and therefore expression of the protein or polypeptide is maximized. The same protease-resistant character is advantageous in mixed formulations that deliberately include a protease, for example, in formulations used for laundry cleaning, or in processing situations where protease will be encountered, for example cell disruptates. It is believed that this tertiary structure also confers aqueous solubility and enhances thermal stability and acid/pH stability on the polypeptide or protein.

In a particular embodiment of the invention, the protein or polypeptide has four α-helical peptides, each linked together by a sequence of 3 to 5 amino acid residues and forms a hydrophobically stabilized tetramer or 4-helix bundle. In another particular embodiment, the protein or polypeptide of the invention has two α-helical peptides linked together by a sequence of 3 to 5 amino acid residues and forms a hydrophobically stabilized dimer or 2-helix bundle.

The polypeptides or proteins of the invention may be biosurfactants in which each α-helical peptide comprises at least one stimuli-responsive amino acid residue.

In one embodiment, the at least one stimuli-responsive amino acid residue is at least one charged amino acid residue such as lysine, histidine, ornithine, glutamic acid, aspartic acid and arginine, especially a histidine residue or a lysine residue, most especially a lysine residue. In some embodiments each α-helical peptide has one lysine residue. In other embodiments, each α-helical peptide has more than one charged residue, where each charged residue is the same. For example, in some embodiments, each α-helical peptide has two, three or four lysine residues, two, three or four histidine residues, two, three or four glutamic acid residues, two, three or four aspartic acid residues or two, three or four arginine residues. In a particular embodiment, the charged amino acid residue is a lysine residue or more than one lysine residue, for example, two, three or four lysine residues per a-helical peptide. The lysine residues may be positioned at any one′ of positions b, c, e, f or g in one or more of the sequences (a b c d d′ e f g) in the α-helical peptide. In some embodiments, a lysine residue is positioned at position b, especially position b of the first sequence (a b c d d′ e f g) in the α-helical peptide.

In another embodiment, the at least one stimuli-responsive amino acid residue is an amino acid residue that has a side chain that can bind a metal ion, for example a histidine residue or a residue containing a carboxylate group such as aspartic acid or glutamic acid. In a particular embodiment, the at least one stimuli-responsive amino acid residue is at least one histidine residue. In some embodiments, each α-helical peptide has one amino acid that can bind a metal ion. In other embodiments, each α-helical peptide has more than one amino acid that can bind a metal ion, for example, two, three or four metal ion binding residues or one metal ion binding residue per sequence (a b c d d′ e f g) in the α-helical peptide. The metal ion binding residue may be present at any one of positions b, c, e, f or g in one or more of the sequences (a b c d d′ e f g) in the α-helical peptide. In some embodiments, the metal ion binding residue is positioned at position b or f. In some embodiments, a metal ion binding residue may be present at position b of one or more sequences (a b c d d′ e f g) or position f of one or more sequences (a b c d d′ e f g). In some embodiments, a metal binding residue is present at position b of one or more of sequence (a b c d d′ e f g) and at position f in one or more other sequences (a b c d d′ e f g) in the α-helical peptide. In a particular embodiment, the metal ion binding residue is a histidine residue.

In another embodiment, the at least one stimuli-responsive amino acid residue results in each sequence (a b c d d′ e f g) in the α-helical peptide having a net positive or negative charge of 1 or 2 at a specified pH. In some embodiments, each sequence in each α-helical peptide has a net positive charge. For example, when the sequence is at a specified pH, if an acidic amino acid residue is present it is protonated and therefore has no charge or if the acidic amino acid residue is charged, the sequence contains more than one basic residue and the basic residues such as lysine, arginine or histidine have a positive charge providing a net positive charge. Alternatively, when the sequence is at a specified pH, any basic residue is not protonated and therefore has no charge or if the basic amino acid is charged, the sequence contain more than one acidic residue and the acidic residues are charged to provide a net negative charge. The pH required to provide the net negative or net positive charge will be determined by the pK₂ of the acidic and/or basic side chains within the residues in the sequence and the local chemical environment, in particular the local dielectric environment.

In some embodiments, each sequence (a b c d d′ e f g) in the α-helical peptide comprises at least one glutamine or asparagine residue and no net charge or each sequence (a b c d d′ e f g) comprises at least one glutamine or asparagine residue and one negative charge.

In some embodiments, each sequence (a b c d d′ e f g) in the α-helical peptide comprises one or two glutamine or asparagine residues, especially two glutamine or asparagine residues, and no net charge. The one or two glutamine or asparagine residues may be in any of positions b, c, e, for g, in the sequence, especially in positions b, c or for b or c and f. In order to provide no net charge, either none of the residues b, c, e, f and g, that are not glutamine or asparagine are positively or negatively charged amino acid residues or if one of b, c, e, f or g is a positively charged amino acid residue, another of b, c, e, f and g is a negatively charged amino acid residue provided that amino acid residues b and c are not both charged amino acid residues.

In other embodiments, each sequence (a b c d d′ e f g) in the α-helical peptide comprises one glutamine or asparagine residue and one residue that is negatively charged, for example, a glutamic acid or aspartic acid residue. The glutamine or asparagine residue may be positioned at any of residues b, c, e, f and g and the negatively charged amino acid residue may be positioned at any of the other positions of b, c, e, f or g. In some embodiments, the glutamine or asparagine residue is at position b or c and the negatively charged amino acid residue is at position f of the sequence. Alternatively, the glutamine or asparagine residue is at position f and the negatively charged amino acid residue is at position b or c, especially c. Each α-helical peptide includes at least two sequences (a b c d d′ e f g) and where two or more different sequences are present in an α-helical peptide, the positions of the glutamine or asparagine residue and the negatively charged residue may be the same or different in each sequence.

The polypeptides and proteins in which each sequence (a b c d d′ e f g) comprises one or two glutamine residues and no net charge or a net negative charge may undergo deamidation to provide a highly anionic polypeptide or protein.

In some embodiments, the polypeptide or protein is part of a fusion protein formed with a second protein, polypeptide or peptide.

In some embodiments, the polypeptide or protein has one of the following sequences:

| | |
|---|---|
| SEQ ID NO: 1: | MD(PS-MKQLADS-LHQLARQ-VSRLEHA-D)₄ |
| SEQ ID NO: 2: | MD(PS-MKQLADS-LHQLARQ-VSRLEHA-D)₂ |
| SEQ ID NO: 3: | MD(PS-AKSLAES-LHSLARS-VSRLEHA-D)₄ |
| SEQ ID NO: 4: | MD(PS-AKSVAES-LHSLARS-VSRLVEHA-D)₄ |
| SEQ ID NO: 5: | MD(PS-AHSVAES-LHSLARS-VSRLVEHA-D)₄ |
| SEQ ID NO: 6: | MD(PS-AHSVAKS-LHSLARS-VSRLVSHA-D)₄ |
| SEQ ID NO: 7: | MD(PS-AHSVAES-LHSLAES-VSELVSHA-D)₄ |
| SEQ ID NO: 8: | MD(PS-AQSVAQS-LAQLAQS-VSQLVSQA-D)₄ |
| SEQ ID NO: 9: | MD(PS-ANSVANS-LANLANS-VSNLVSNA-D)₄ |
| SEQ ID NO: 10 | MD(PS-AQSVAES-LAQLAES-VSELVSQA-D)₄ |
| SEQ ID NO: 11 | MD(PS-ANSVAES-LANLAES-VSELVSNA-D)₄ |

In a particular embodiment, the polypeptide or protein is a biosurfactant polypeptide or protein that has SEQ ID NO:1.

In yet another aspect of the invention there is provided an α-helical peptide derived from cleavage of cleavable bonds in the linking sequence of the polypeptide or protein of the invention or a synthetic equivalent of the α-helical peptide. In this aspect, the α-helical peptides comprise a sequence of amino acid residues:
X₁-(a b c d d′ e f g)ₙ-X₂
   wherein a, b, c, d, d′, e, f, g and n are defined above, X₁ and X₂ are each independently absent or are an amino acid residue or sequence of residues found in the linking sequence of the protein or polypeptide of the invention and wherein the number of amino acid residues X₁+X₂ is 0 to 11.

For example, when the cleavable linking sequence comprises the acid cleavable sequence D-P, X₁ will comprise a proline residue (P) and X₂ will comprise an aspartic acid residue (D). X₁ and/or X₂ may also comprise other amino acid residues that appear in the linking sequence. For example, when the acid cleavable linking sequence comprises or consists of the sequence D-P-S, X₁ will comprise or consist of P-S and X₂ will comprise or consist of D. Alternatively, in some embodiments, the cleavage conditions used may result in loss of linking sequence residues from the α-helical peptide. For example, under acid cleavage conditions, the D of the D-P bond may be cleaved from the peptide resulting in X₂ being absent.

In another embodiment where the linking sequence contains a base cleavable bond, X₁ and X₂ may comprise amino acid residues from each side of the cleavable bond. For example, when the base cleavable bond is a asparagine-glycine bond, X₁ will comprise a glycine residue and X₂ will comprise an asparagine residue.

In yet another embodiment, where the linking sequence comprises an enzyme cleavable bond, X₁ and X₂ may comprise amino acid residues from each side of the cleavable bond. For example, if the linking sequence is cleavable by tobacco etch virus protease, X₁ may comprise a glycine or serine residue and X₂ may comprise an E-N-L-Y-F-Q sequence.

In some embodiments, the cleavage conditions may alter the amino acid residues in the (a b c d d′ e f g) sequence. For example, under strong acid cleavage conditions, one or more glutamine or asparagine residues in the sequence may be deamidated to form glutamic acid or aspartic acid residues.

In some embodiments, the α-helical peptides may be synthetically produced by methods known in the art, for example, solid phase synthesis, and X₁ and X₂ may be included or omitted from the synthesis.

In particular embodiments, the α-helical peptides have one of the following sequences:

| | |
|---|---|
| SEQ ID NO: 12 | PS-MKQLADS-LHQLARQ-VSRLEHA-D |
| SEQ ID NO: 13 | PS-MKELADS-LHELARE-VSRLEHA-D |
| SEQ ID NO: 14 | PS-MKELADS-LHQLARQ-VSRLEHA-D |
| SEQ ID NO: 15 | PS-MKQLADS-LHELARQ-VSRLEHA-D |
| SEQ ID NO: 16 | PS-MKQLADS-LHQLARQ-VSRLEHA-D |
| SEQ ID NO: 17 | PS-MKELADS-LHELARQ-VSRLEHA-D |
| SEQ ID NO: 18 | PS-MKELADS-LHQLARE-VSRLEHA-D |
| SEQ ID NO: 19 | PS-MKQLADS-LHELARE-VSRLEHA-D |
| SEQ ID NO: 20 | PS-AKSLAES-LHSLARS-VSRLEHA-D |
| SEQ ID NO: 21 | PS-AKSVAES-LHSLARS-VSRLVEHA-D |
| SEQ ID NO: 22 | PS-AHSVAES-LHSLARS-VSRLVEHA-D |
| SEQ ID NO: 23 | PS-AHSVAKS-LHSLARS-VSRLVSHA-D |
| SEQ ID NO: 24 | PS-AHSVAES-LHSLAES-VSELVSHA-D. |
| SEQ ID NO: 25 | PS-AQSVAQS-LAQLAQS-VSQLVSQA-D |
| SEQ ID NO: 26 | PS-AESVAES-LAELAES-VSELVSEA-D |
| SEQ ID NO: 27 | PS-ANSVANS-LANLANS-VSNLVSNA-D |
| SEQ ID NO: 28 | PS-ADSVADS-LADLADS-VSPLVSDA-D |
| SEQ ID NO: 29 | PS-AQSVAES-LAQLAES-VSELVSQA-D |
| SEQ ID NO: 30 | PS-AESVAES-LAELAES-VSELVSEA-D |
| SEQ ID NO: 31 | PS-ANSVAES-LANLAES-VSELVSNA-D |
| SEQ ID NO: 32 | PS-ADSVAES-LADLAES-VSELVSDA-D |

In a particular embodiment, the α-helical peptide is a peptide of SEQ ID NO:12.

In another aspect of the invention there is a composition comprising at least one polypeptide or protein of the invention and at least one α-helical peptide of the invention.

In some embodiments, the composition is in solid form and suitable for reconstitution in a solvent. In other embodiments, the composition further comprises a solvent. Suitable solvents may be aqueous or non-aqueous and are preferably suitable for foam formation. In particular embodiments, the solvent is an aqueous solvent. Examples of suitable solvents include water, buffer, acetonitrile, water and alcohol mixtures such as aqueous methanol, aqueous ethanol and aqueous isopropanol.

In some embodiments, the composition is prepared by mixing the protein or polypeptide and the α-helical peptide in given proportions. Suitable proportions of protein or polypeptide to α-helical peptides include 1:10 to 10:1, for example 1:9 to 9:1, 1:8 to 8:1, 1:7.5 to 7.5:1, 1:7 to 7:1, 1:6 to 6:1, 1:5 to 5:1, 1:4 to 4:1, 1:3 to 3:1, 1:2.5 to 2.5:1, 1:2 to 2:1 and 1:1.

In some embodiments, the composition is prepared by cleavage of cleavable bonds in the polypeptide or protein of the invention. In this embodiment, the cleavage may be stopped before completion of the cleavage to provide a given proportion of polypeptide or protein to α-helical peptide. The reaction may be followed by chromatography, such as reversed phase (RP)-HPLC coupled with mass spectrometry for analysis of cleavage products and microchemical changes such as deamidation reactions. When the reaction has progressed to the desired amount of cleavage and/or maximum amount of microchemical changes desired, the reaction may be stopped, for example, by neutralisation of the reaction mixture. In this embodiment, the composition may comprise other cleavage products such as polypeptides having a reduced number of α-helical peptides than the starting polypeptide or protein. For example, where the starting polypeptide or protein of the invention comprises three cleavable linking sequences and four α-helical peptides, the composition may comprise not only the starting polypeptide or protein but also polypeptides comprising two cleavable linking sequences and three α-helical peptides and/or one cleavable linking sequence and two α-helical peptides. In some embodiments, where the α-helical peptide comprises glutamine or asparagine residues, the composition may also comprise other peptides that are derived from the original α-helical peptides within the protein or polypeptide. For example, the glutamine or asparagine residues may deamidate to provide glutamic acid or aspartic acid residues.

### Methods of Manufacture and Purification

The polypeptides and proteins of the present invention may be manufactured in high yields using standard microbial culture technology, genetically engineered microbes and recombinant DNA technology as known in the art (Sambrook and Russell, Molecular cloning: A Laboratory Manual (3^{rd} Edition), 2001, CSHL Press).

The genetically engineered microbes contain a polynucleotide sequence that comprises a nucleotide sequence that encodes the polypeptide or protein. The nucleotide sequence is operably linked to a promoter sequence.

The microbes may be any microbes suitable for use in culturing processes such as fermentation. Examples of suitable microbes include E. coli, Saccharomyces cerevisiae, Bacillis Subtilis and Piccia pastoris, especially E. coli.

In a particular embodiment, the culturing process is fermentation. During the culturing process, the microbes express the polypeptide or protein.

Without wishing to be bound by theory, the protein or polypeptide produced is able to fold to give a defined tertiary structure having a substantially hydrophilic surface and substantially hydrophobic core in the cytoplasm of the microorganism thereby increasing resistance to proteolysis and affording high levels of expressed protein or polypeptide to be isolated.

Once culturing is complete, such as fermentation, the microbial cells may be further treated in the culture medium, for example, the fermentation broth, or may be isolated and stored or re-suspended in the same or different media. Cells may be isolated by commonly used techniques such as centrifugation or filtration.

Optionally the cells may undergo a cell-conditioning step after cell recovery. For example, the cells may be collected and re-suspended in water or buffered solution prior to storage or use.

After culturing, the microbial cells are disrupted to provide a disruptate composition comprising soluble proteins and cell debris. Cell disruption may be achieved by means known in the art including mechanical means and non-mechanical means. Small scale disruption may be achieved by methods such as sonication or homogenization. Large scale disruption may be achieved by mechanical means such as bead milling, homogenization and microfluidization, or non-mechanical means including physical means such as decompression, osmotic shock and thermolysis; chemical means such as antibiotics, chelating agents, chaotropes, detergents, solvents, hydroxide and hyperchlorite; and enzymatic means such as lytic enzymes, autolysis and cloned phage lysis. In practice, for large scale disruption of bacterial cells such as E. coli, mechanical means are currently used and thermolysis is avoided as it must be conducted at temperatures that typically cause irreversible protein denaturation and aggregation.

Optionally after the cell disruption step, solid cell debris is removed by techniques known in the art such as centrifugation or filtration. Removal of cell debris provides a solution of soluble cell proteins that includes the protein or polypeptide of interest.

Purification of the proteins and polypeptides having a tertiary structure that folds to give a substantially hydrophobic core and a substantially hydrophilic surface from other contaminating cell proteins and polypeptides may be achieved by treating the cell disruptate, either directly from cell disruption or clarified by removal insoluble cell debris, with a kosmotropic salt in an amount suitable to salt-out cell derived contaminants but salt-in the protein or polypeptide molecule of the invention.

Surprisingly, it has been found that the proteins and polypeptides of the invention remain soluble and are salted-in in the presence of moderate amounts of kosmotropic salts that are sufficient to salt-out and precipitate many contaminating cell proteins and polypeptides. Additionally, it has been surprisingly found that the proteins and polypeptides of the invention can be salted-in even under elevated temperatures in the presence of moderate amounts of kosmotropic salts.

The kosmotropic salt may be formed from a kosmotropic ion. Examples of kosmotropic ions include sulphate (SO₄²⁻), carbonate (CO₃²⁻), phosphate (PO₄³⁻), lithium (Li⁺), fluoride (F⁻), calcium (Ca⁺⁺) and acetate (CH₃COO⁻). The counterion in the salt may be any suitable counterion of opposite charge. Examples of suitable kosmotropic salts include ammonium sulphate, sodium sulphate, potassium sulphate, sodium carbonate, sodium bicarbonate, potassium carbonate, potassium bicarbonate, ammonium carbonate, sodium phosphate, potassium phosphate and ammonium phosphate. In some embodiments the kosmotropic salt is selected from ammonium sulphate, sodium sulphate, potassium sulphate, especially ammonium or sodium sulphate.

The amount of kosmotropic salt is an amount suitable to precipitate contaminating proteins or polypeptides but not the proteins or polypeptides of interest. This amount can readily be determined by those skilled in the art by exposing a sample of the cell disruptate, with or without clarification, to a range of salt concentrations, separating the precipitate and supernatant and analyzing the supernatant to determine the amount of contaminating proteins in the supernatant and pellet by SDS-PAGE or HPLC. The suitable amount of kosmotropic salt allows salting-out and precipitation of most or all of the cell contaminants but minimal or no precipitation of the protein or polypeptide of interest. In some embodiments, very high purity of the polypeptide or protein may be achieved by salting-out all contaminants even though this may lead to incomplete salting-in of the polypeptide or protein and some reduction in yield.

In some embodiments, the amount of kosmotropic salt is in the range of 0.2 M and 2.0 M. In some embodiments, the amount of kosmotropic salt is in the range of 0.2 M and 0.5 M for example, about 0.25 M. In other embodiments, the amount of kosmotropic salt is in the range of 0.5 M and 2.0 M, for example, 1.0 M and 2.0 M, especially about 1.5 M.

The pH of the disruptate may also affect the precipitation of cell contaminants from the solution and the solubility of the protein or polypeptide being manufactured. At its isoelectric pH or point (pI), a protein has no net charge and therefore charge repulsion is reduced and aggregation of proteins and precipitation may occur. Most proteins have a pI between 6.0 and 7.5 which may be exploited to provide aggregation or precipitation of contaminants while acid-rich proteins or polypeptides of the present invention remain in solution. In some embodiments, especially where the protein or polypeptide being manufactured has a pI above 6, the precipitation or salting-out step may be performed at pH between 3 and 6, especially 3.5 to 5.0, 3.5 to 4.5, more especially about 4. At pH between 3.0 and 5.0, for example between 3.0 and 4.0, acid-induced denaturation of contaminant proteins or polypeptides may also occur causing partial unfolding of tertiary structure or a reduction in structural stability.

After treatment with the kosmotropic salt, the precipitate containing cell contaminants and the supernatant containing the polypeptide or protein may be separated. The separation may be achieved by methods known in the art such as gravity sedimentation, centrifugation or filtration.

In some embodiments, the purification step is performed at atmospheric pressure and elevated temperature, above 45° C. or especially above 80° C. For example, the elevated temperature may be in the range of 50° C. to 100° C., 60° C. to 100° C., 70° C. to 100° C., 80° C. to 100° C. or 85° C. to 100° C. In some embodiments, the elevated temperature is in the range of 85° C. to 98° C., especially about 90° C. to 95° C. In some embodiments, the purification step is performed at elevated pressure and elevated temperature, for example, by autoclaving the composition comprising the polypeptide or protein and other cell based protein, polypeptide and/or peptide contaminants. For example, autoclaving may be performed at a pressure of 1-2 atmospheres and 100-130° C., especially a pressure of about 2 atmospheres and about 121° C. In other embodiments, the purification step may be performed at reduced pressure and therefore reduced temperature, for example 0.5 atmospheres and a temperature below 60° C.

While thermolysis is a known technique for cell disruption, the heating of bacterial cells to cause disruption and release of cell contents is rarely useful. Thermolysis often results in only partial release of cell contents and if higher temperatures are used, such as above 60° C., the proteins and polypeptides of interest are denatured by the heat.

Surprisingly, the present inventors have found that for proteins and polypeptides having a folded tertiary structure with a substantially hydrophobic core and a substantially hydrophilic surface, for example, a folded helix structure such as a helix bundle, cell disruption by heat treatment can be performed in the presence of a kosmotropic salt resulting not only in cell disruption but also salting-out of the cell based proteins, polypeptides and peptides while the protein or polypeptide having folded tertiary structure is salted-in the solution. The heat treatment may be performed at atmospheric pressure and a temperature of at least 60° C., especially 80° C. to 100° C. or 85° C. to 100° C., more especially in the range of 85° C. to 98° C., for example 90° C. to 95° C.; or at elevated pressure and temperature, such as by autoclaving; or at reduced pressure and temperature. This heat treatment may be performed directly on the fermentation broth. Alternatively, the microbial cells may be isolated from the cell broth, optionally treated further by cell conditioning, and optionally stored before heat treatment for cell disruption.

The kosmotropic salt may be formed from a kosmotropic ion. Examples of kosmotropic ions include sulphate (SO₄²⁻), carbonate (CO₃²⁻), phosphate (PO₄³⁻), lithium (Li⁺), fluoride (F⁻), calcium (Ca⁺⁺) and acetate (CH₃COO⁻). The counterion in the salt may be any suitable counterion of opposite charge. Examples of suitable kosmotropic salts include ammonium sulphate, sodium sulphate, potassium sulphate, sodium carbonate, sodium bicarbonate, potassium carbonate, potassium bicarbonate, ammonium carbonate, sodium phosphate, potassium phosphate, ammonium phosphate, calcium chloride and lithium chloride. In some embodiments the kosmotropic salt is selected from ammonium sulphate, sodium sulphate, potassium sulphate, calcium chloride, lithium chloride, especially ammonium or sodium sulphate or calcium chloride

The amount of kosmotropic salt is an amount suitable to precipitate the contaminating proteins and polypeptides but salt-in the proteins and polypeptides of interest. This amount may be determined by those skilled in the art by exposing a sample of the cells in a suitable solvent, such as fermentation media, water or buffer, to a range of salt concentrations and heating the cell composition to at least 60° C., separating the precipitate and supernatant and analyzing the supernatant to determine the amount of contaminating proteins in the supernatant and the pellet by SDS-PAGE. The suitable amount allows salting-out of most or all of the cell contaminants but minimal or no precipitation of the protein or polypeptide of interest. In some embodiments, a suitable amount of kosmotropic salt is in the range of 0.2 M and 2.0 M. In some embodiments, the amount of kosmotropic salt is in the range of 0.2 M and 0.5 M for example, about 0.25 M. In other embodiments, the amount of kosmotropic salt is in the range of 0.5 M and 2.0 M, for example, 1.0 M and 2.0 M, especially about 1.5 M.

In some embodiments, the high concentration of kosmotropic salt is removed from the supernatant containing the protein or polypeptide, for example, by dialysis or ultrafiltration. In some embodiments, the concentration of kosmotropic salt is reduced by dilution of the supernatant.

In some embodiments, the supernatant containing the purified protein or polypeptide may be stored or used directly in an industrial application. Alternatively, the protein or polypeptide may be isolated from the supernatant as a solid by a second precipitation step. The solid protein or polypeptide may be more suitable for storage and/or transport or allow resolubilization in a more appropriate solvent for industrial use.

The precipitation of the protein or polypeptide may be achieved by many protein precipitation methods, including metal ion precipitation, acid precipitation, salting-out with a kosmotropic salt or concentration of the solution by evaporation.

In some embodiments, the protein or polypeptide may be precipitated from the supernatant by increasing the concentration of kosmotropic salt. A person skilled in the art could readily determine a suitable amount of kosmotropic salt to precipitate the protein or polypeptide by exposing the protein or polypeptide to a range of concentrations of salt, separating precipitate and supernatant and analyzing each of the precipitate and supernatant for the protein or polypeptide by SDS-PAGE or HPLC.

In one embodiment, kosmotropic salt may be increased above 1.5 M, for example 1.5 M to 4 M, 2 M to 4 M or 3 M to 4 M, especially about 3.5 M.

In some embodiments, the protein or polypeptide may be successfully salted-out of the solution using these high amounts of kosmotropic salts at pH ranges between 3 and 7. Adjustment of pH may not be required after the initial precipitation of impurities.

Another option for precipitation of the protein or polypeptide after removal of the cell impurities is to concentrate the supernatant containing the protein or polypeptide and kosmotropic salt. Concentration may be achieved by heating the solution to elevated temperature causing evaporation of the supernatant liquid. Suitable elevated temperatures are above 80° C., for example 80° C. to 100° C., 85° C. to 100° C. or 90° C. to 100° C. Alternatively, evaporation of the supernatant liquid may be achieved under a stream of gas, such as air or nitrogen gas. Concentration of the solution results in an increase in the concentration of kosmotropic salt which leads to precipitation of the protein or polypeptide.

Once the protein or polypeptide has been precipitated and isolated, the kosmotropic salt may be isolated from the supernatant and recycled.

In some embodiments, the protein or polypeptide may also be precipitated by metal ion precipitation. Suitable metal ions include Mn⁺⁺, Fe⁺⁺, Co²⁺, Cu²⁺, Zn⁺⁺, Ni⁺⁺ and Cd⁺⁺, which strongly bind to carboxylic acids and to nitrogenous compounds such as amines and heterocycles; Ca⁺⁺, Ba⁺⁺, MgI⁺ and Pb⁺⁺, which bind to carboxylic acids but not significantly to nitrogenous compounds, and Ag⁺, HgI⁺ and Pb⁺⁺, which strongly bind to sulfhydryl groups. The metal ion selected for use may be dictated by the sequence of amino acids in the protein or polypeptide. For example, one of Mn⁺⁺, Fe⁺⁺, Co⁺⁺, Cu⁺⁺, Zn⁺⁺, Ni⁺⁺ and Cd⁺⁺ may be suitable for precipitation of protein or polypeptide containing histidine residues, glutamic acid residues or aspartic acid residues whereas Ag⁺, HgI⁺ or Pb⁺⁺ may be more suitable for protein or polypeptide containing a number of cysteine residues.

Metal ion precipitation of the protein or polypeptide is more effective if the kosmotropic ion from the purification step (precipitation of cell based contaminants) is diluted to below 0.5 M or removed from solution.

The amount of metal ion used to give precipitation will vary depending on the reaction conditions, such as pH and the concentration of kosmotropic salt present in the solution. In some embodiments, the amount of metal ion used to precipitate the polypeptide or protein is in the range of 1-20 mM. Removal of the kosmotropic salt or dilution to below 0.2 M may allow precipitation of the protein or polypeptide by a metal ion in an amount in the range of 1.0 mM to 10 mM, especially 2 to 9 mM, 3 to 8 mM or 4 to 7 mM, especially about 5 mM.

The metal ion precipitation is most efficient at neutral or mildly alkaline pH. In some embodiments, the pH of the solution is adjusted to be in a range of 6 to 10, especially 7 to 8.5, more especially 7.0 to 7.5.

In some embodiments, particularly proteins or polypeptides comprising histidine, aspartic acid and/or glutamic acid residues in the sequence, metal ion precipitation with Zn⁺⁺ or Ni⁺⁺, especially Zn⁺⁺, is used. In particular embodiments, the protein or polypeptide comprises histidine residues and the metal ion is Zn⁺⁺. This method is particularly useful for proteins or polypeptides of SEQ ID NO:1-7.

In some embodiments, the protein or polypeptide may be isolated by acid precipitation. In some embodiments in order to successfully precipitate the protein or polypeptide, the concentration of kosmotropic salt from the purification step will need to be reduced, for example, to below 0.3 M, or to about 0.1 M. After removal or dilution of the kosmotropic salt, the pH of the solution may be adjusted to within the range of 2 to 5, especially 3 to 4 to effect precipitation of the protein or polypeptide.

The precipitated protein or polypeptide may be isolated by methods known in the art such as centrifugation or filtration. The collected solid protein or polypeptide may then be dried by means known in the art, such as heating the solid, drying at reduced pressure or using a rotary drum vacuum filtration system.

In some embodiments, the protein or polypeptide has surfactant properties. In some embodiments, the protein or polypeptide comprises multiple α-helical peptides, each having surfactant properties. The multiple α-helical peptides are linked by a linking, sequence of amino acids that includes a cleavable amino acid sequence. The cleavable amino acid sequence may be enzyme cleavable, acid cleavable or base cleavable.

In some embodiments of this method, the polypeptide comprises at least two α-helical peptides linked by a linking sequence of 3 to 11 amino acid residues, wherein each a-helical peptide comprises a sequence of amino acid residues:
(a b c d d′ e f g)ₙ
   wherein n is an integer from 2 to 12;
   amino acid residues a and d are hydrophobic amino acid residues;
   amino acid residue d′ is absent or is a hydrophobic amino acid residue;
   at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
   amino acid residue g is any amino acid residue.

In some embodiments in which the linking sequence includes a cleavable linker, the protein or polypeptide may be exposed to an enzyme suitable to cleave the linker or to acid or base conditions suitable to cleave the linker. The cleavage of a cleavable linker may be performed at any time in the process but suitably after purification of the protein or polypeptide by removal of cell debris and contaminating proteins. In this embodiment, a-helical peptides of the invention may be produced. An alternative method of producing the α-helical peptides of the invention is by peptide synthesis, such as solid or liquid phase synthesis as known in the art.

In some embodiments, the protein or polypeptide contains an acid cleavable linker that comprises an acid cleavable peptide bond. A suitable acid cleavable bond is a D-P bond. The bond is cleaved by exposing the protein or polypeptide to acid of a suitable pH at a suitable temperature for a suitable period of time [M. Landon cleavage at aspartyl-prolyl bonds, in selective cleavage by chemical methods, 1977, p 145-149] to cleave the acid cleavable bond. For example, mild conditions include acetic acid at pH 2.5 at 40° C. for 24-120 hours, while more vigorous conditions use formic acid for a shorter period of time. In a particular method, the polypeptide or protein is incubated for 24-48 hours in dilute HCl (60 mM) at a temperature of 60° C. or for a shorter time at higher temperature, for example, 90-120° C. for 1-3 hours. In some embodiments, cleavage of the acid cleavable linker may be performed concomitantly with the purification step (step iv), optionally at a temperature of at least 60° C.

In some embodiments, the protein or polypeptide comprises an enzyme cleavable linker sequence. The sequence may be designed to be cleaved by a specific protease enzyme, such as trypsin, chymotrypsin, elastase or Tobacco Etch Virus protease.

In embodiments where the protein or polypeptide comprises one or more cleavable linking sequences, the rest of the protein or polypeptide is designed to have reduced potential for cleavage under the cleavage conditions. For example, the α-helical peptides may be designed such that they lack cleavable bonds and/or may be designed to have lower rates of non-specific cleavage, for example by replacing Asp residues with Glu.

Cleavage of the cleavable linkers results in multiple peptides being produced. This method allows access to peptides in high yield from fermentation processes without the need for use of a fusion protein carrier.

In some embodiments, the protein or polypeptide is rich in amino acid residues having an amide in their side chain, such as glutamine and/or asparagine. While these amino acid residues are polar, they are not charged. However, after expression of the protein or polypeptide and optionally before, during or after cleavage of cleavable linkers, the amide groups may be deamidated to produce carboxylic acids leading to a protein or polypeptide or peptide rich in anionic charge. Peptides having an abnormally high negative charge may express poorly by themselves in recombinant cell lines.

Deamidation of glutamine and asparagine residues in a protein, polypeptide or peptide may be achieved at acidic pH, for example, in the pH range of 1 to 3. Asparagine residues may also be cleaved at a basic pH, for example, in the pH range of 7.5 to 9. In some embodiments, deamidation may be performed at elevated temperature, for example above 45° C. to 100° C., 50° C. to 100° C., 60° C. to 100° C., 70° C. to 100° C., 85° C. to 100° C., especially about 90° C. to 95° C. In some embodiments, where peptides are produced by the use of acid cleavable linkers, deamidation and acid cleavage of the linking sequence may occur concomitantly.

In one aspect of the invention, the thermal and acid stability of the proteins or polypeptides folded to provide a tertiary structure with a substantially hydrophobic core and a substantially hydrophilic surface may be utilized as carriers in the preparation and isolation of other proteins, polypeptides or peptides of interest. The protein or polypeptide carrier and the second protein, polypeptide or peptide may be produced by the method of the invention in the form of a fusion protein comprising the protein or polypeptide carrier linked to the second protein, polypeptide or peptide by a cleavable linker.

Alternatively, the polypeptide or protein may be incorporated into the production of a known fusion protein comprising a protein and carrier, such that upon cleavage of the fusion biosurfactant-protein-carrier conjugate, a composition of protein and biosurfactant can be achieved. This embodiment may be particularly useful for obtaining a composition comprising an enzyme, such as a protease enzyme useful in laundry detergent, and a biosurfactant.

In this method, the polynucleotide comprises not only a first nucleotide sequence encoding the protein or polypeptide optionally as a carrier but also a second nucleotide encoding the second protein, polypeptide or peptide and a sequence encoding a cleavable linker. The first and second nucleotide sequences being linked directly such that expression produces a fusion protein comprising the protein or polypeptide linked to the second protein, polypeptide or peptide by a cleavable linker.

The cleavable linker may be an enzyme cleavable linker, acid cleavable linker or base cleavable linker. Suitable enzyme cleavable linkers are those cleaved by proteases such as subtilisin, trypsin, chymotrypsin, elastase and Tobacco Etch Virus protease (TEVp), especially TEVp. Suitable acid cleavable linkers include sequences comprising a D-P bond.

In some embodiments, the protein or polypeptide of the invention does not include any cleavable bonds such that the only cleavable bond in the fusion protein is in the linking sequence between the protein or polypeptide and the second protein, polypeptide or peptide. In some embodiments, the cleavable linker between the protein or polypeptide and the second protein, polypeptide or peptide is an enzyme cleavable linker such as a protease cleavable linker. In a particular embodiment, the enzyme cleavable linker is cleavable by TEVp and comprises one of the sequences E-N-L-Y-F-Q-G or E-N-L-Y-F-Q-S.

In some embodiments, the cleavable linker between the polypeptide or protein and the second protein, polypeptide or peptide is cleaved by an enzyme at a faster rate than the rate of non-specific cleavage of the protein or polypeptide. In some embodiments, the second protein, polypeptide or peptide is an enzyme useful in laundry wash applications and the linker between the protein or polypeptide biosurfactant and the enzyme is cleavable by protease enzymes useful in laundry applications. In a particular embodiment, the rate of cleavage of the cleavable linker between the enzyme and the protein or polypeptide biosurfactant is faster than the rate of non-specific protease cleavage of the protein or polypeptide biosurfactant under selected pre-wash, wash or process conditions. This embodiment is particularly useful for providing laundry detergent comprising a biosurfactant and enzymes suitable for laundry use.

In some embodiments, the protein or polypeptide includes cleavable linking sequences and the cleavable linking sequences are cleaved by a method that differs from the method used to cleave the fusion protein. For example, the fusion protein may include an enzyme cleavable linker, such as a TEVp or other protease cleavable linker, between the protein or polypeptide and the second protein, polypeptide or peptide, and the protein or polypeptide includes acid or base cleavable linking sequences between α-helical peptides. Conversely, the cleavable linker between the protein or polypeptide and the second protein is an acid or base cleavable linker and the cleavable linking sequences within the protein or polypeptide carrier are enzyme cleavable linkages. Another option is that one of the cleavable linkers is acid cleavable and the other is base cleavable.

In some embodiments, the cleavable linker between the protein or polypeptide and the second protein, polypeptide or peptide and the cleavable linking sequence between the a-helical peptides of the protein or polypeptide are the same. For example, all of the cleavable linkages are acid or base cleavable or are cleavable by the same enzyme.

The fusion protein may be produced in culture and recovered and purified by the method described above. Alternatively, the fusion protein may be produced by fermentation, recovered and cleaved. The protein or polypeptide and second protein, polypeptide or peptide could then be purified separately or used in a single composition.

The second protein, polypeptide or peptide may be any protein, polypeptide or peptide of interest, especially a protein, polypeptide or peptide that is difficult to produce in commercially viable quantities as a single entity by recombinant technology or is difficult to synthesize by standard peptide synthesis techniques. Examples of proteins, polypeptides or peptides include, but are not limited to, antibodies, hormones, enzymes, such as proteases, antimicrobial peptides, peptides selected by phage display or natural sequences for use in materials synthesis or surface binding or those used in industrial, environmental, food or medical products or processes.

In one embodiment of the invention, the second protein, polypeptide or peptide is an antimicrobial peptide (AMP). Antimicrobial peptides are often short peptide sequences lacking tertiary structure and have broad antimicrobial activity including antibacterial and antifungal activity. In some embodiments, the antimicrobial peptide is a cationic antimicrobial host defense peptide (HDP), such as IDR1, MX266 (MBI-226, omiganin), LL37, CRAMP, HHC-10, E5 and E6. Other examples of AMPs that may be produced by the present invention include PAC-113, magainins, alamethicin, pexiganan, MSI-78, MSI-843, MSI-594, polyphemusin, human antibacterial peptide, cathelicidins, defensins and protegrins.

In another embodiment, the second protein, polypeptide or peptide is an enzyme, especially an enzyme useful in laundry detergents such as protease, lipases, amylases and cellulases.

In yet another embodiment, the second protein, polypeptide or peptide is a peptide useful in materials applications such as the manufacture of metallic nanostructures, such as metal alloy ferromagnetic nanostructures [Reiss et al., Nano Letters, 2004, 4, 1127-1131] or in binding of semiconductors [Peelle et al., Langmuir, 2005, 21, 6929-6933]. For example, the second protein, polypeptide or peptide may be designed to bind semiconductors such as CdS, CdSe, ZnS and ZnSe and may include multiple histidine (H6), tryptophan (W6), cysteine (C6) or methionine residues (M6) or may include histidine, cysteine, tryptophan or methionine residues alternating with another residue, especially glycine and basic amino acid residues, in short peptides having 6-10 amino acid residues.

In some embodiments, the fusion protein is cleaved and the protein or polypeptide carrier and the second protein, polypeptide or peptide are separated.

In other embodiments, the protein or polypeptide carrier and second protein, polypeptide or peptide are cleaved and retained in the same composition without separation. This embodiment may be particularly useful for preparing compositions containing a protein, polypeptide or peptide together with a biosurfactant protein, polypeptide or peptide. For example, a composition comprising a stimuli-responsive protein, polypeptide or peptide biosurfactant and an antimicrobial peptide may be produced. Such a composition may be useful in personal care products, cleaning products, laundry detergents, and medicinal compositions such as antimicrobial topical or rinse treatments for the skin, nose, mouth, throat or vagina. In another example, a composition comprising a stimuli-responsive protein, polypeptide or peptide biosurfactant and one or more enzymes, such as a protease, lipase, amylase or cellulase, may be produced. Such a composition may be useful in laundry detergents.

In another aspect of the invention, there is provided a method of purifying a polypeptide or protein that has a folded tertiary structure with a substantially hydrophobic core and a substantially hydrophilic surface; said method comprising the steps of:
i) treating a composition comprising the polypeptide or protein and other cell based protein, polypeptide and/or peptide contaminants with a kosmotropic salt in an amount suitable to salt-out the contaminants to form a precipitate and to salt-in the polypeptide or protein in solution; and
ii) separating the precipitate from the solution containing the polypeptide or protein.

In some embodiments, step i) is performed at atmospheric pressure and a temperature above 45° C., for example, at least 60° C., especially at a temperature in the range of 85° C. to 100° C., more especially about 90° C. to 95° C. In some embodiments, the purification step is performed at elevated pressure and elevated temperature, for example, by autoclaving the composition comprising the polypeptide or protein and other cell based protein, polypeptide and/or peptide contaminants. For example, autoclaving may be performed at a pressure of 1-2 atmospheres and 100-130° C., especially a pressure of about 2 atmospheres and about 121° C. In other embodiments, the purification step may be performed at reduced pressure and therefore reduced temperature, for example 0.5 atmospheres and a temperature below 60° C. In some embodiments, the kosmotropic salt is a sulphate, especially ammonium sulphate or sodium sulphate. In some embodiments, the amount of kosmotropic salt is in the range of 0.2 M to 1.5 M. In some embodiments, the amount of kosmotropic salt is in the range of 0.2 M and 0.5 M for example, about 0.25 M. In other embodiments, the amount of kosmotropic salt is in the range of 0.5 M and 2.0 M, for example, 1.0 M and 2.0 M. In some embodiments, the folded tertiary structure is a helix bundle, especially a four helix bundle.

### Modulation of Foam Stability

In one aspect the present invention provides a method of modulating the stability of foam comprising a protein or polypeptide biosurfactant at a liquid-gas interface, wherein the biosurfactant comprises at least two α-helical peptides, each α-helical peptide linked by a sequence of 3 to 11 amino acid residues wherein each α-helical peptide comprises a sequence of amino acid residues:

(a b c d d′ e f g)ₙ
wherein n is an integer from 2 to 12;
amino acid residues a and d are hydrophobic amino acid residues;
amino acid residue d′ is absent or is a hydrophobic amino acid residue; and
amino acid residues b, c, e, f and g are any amino acid residue,
and wherein each α-helical peptide comprises at least one stimuli-responsive amino acid residue, said method comprising the step of:
i) exposing the biosurfactant to a stimulus that alters the zeta potential and/or surface charge of the biosurfactant at the liquid-gas interface or the metal ion binding of the biosurfactant or hydration structure of the biosurfactant at the liquid-gas interface.

In this aspect the proteins or polypeptides of the invention have surfactant properties such as affinity for a liquid-gas interface and amphiphilic character.

In some embodiments, the foam may optionally comprise an emulsion, which is a dispersion of oil in water.

In some embodiments, the foam further comprises an α-helical peptide of the invention in addition the polypeptide or protein of the invention, particularly an α-helical peptide that comprises at least one stimuli-responsive amino acid residue. Without wishing to be bound by theory, it appears that the combination of polypeptide or protein of the invention and the smaller α-helical peptide of the invention increases the rate of interfacial tension decrease at the liquid-gas interface and thereby improves foamability.

In particular embodiments where an α-helical peptide is included in the foam in addition to the polypeptide or protein of the invention, the α-helical peptide has an amino acid sequence that is the same as the amino acid sequence of an α-helical peptide in the polypeptide or protein or a sequence that is derived from microchemical modifications, where any asparagine and/or glutamine residues are deamidated.

The stability of the foam may be modulated by preventing formation of the foam, stabilizing the foam or destabilizing the foam, or a combination of these. For example, a solution containing a biosurfactant and a stimulus may prevent formation of a foam, stabilize the formation of a foam, maintain the foam, destabilize the foam or collapse the foam. In other embodiments, the step of exposing the biosurfactant to the stimulus is repeated, optionally multiple times. For example, at one time a solution comprising biosurfactant and a stimulus prevents formation of a foam and at a second time, a stimulus is added to the solution that results in the formation and maintenance of a stable foam or at one time, in the presence of a biosurfactant and a stimulus a stable foam is formed and at a second time, a stimulus is added and the foam is destabilized and collapses. The present invention allows a stable foam to be switched on and/or off in a controlled manner.

In some embodiments, the stimulus may be added to the bulk solution from which foam is formed or added to the bulk solution after foam is formed. In other embodiments, the stimulus may be added by diluting or replacing the bulk solution. In some embodiments the stimulus is added to the foam. Exposure of foam to the stimulus may occur in a localized manner causing the foam to collapse locally and allowing surrounding parts of the foam to come into contact with the stimulus ultimately resulting in collapse of the entire foam.

In one embodiment, the at least one stimuli-responsive amino acid residue is a charged amino acid residue. In some embodiments, the charged residue is selected from lysine, histidine, arginine, ornithine, aspartic acid or glutamic acid.

In a particular embodiment, the stimuli-responsive amino acid residue is a lysine residue. In some embodiments, each α-helical peptide in the polypeptide or protein has one lysine residue. In other embodiments, each α-helical peptide in the polypeptide or protein has more than one lysine residue, for example, two, three or four lysine residues per α-helical peptide. The lysine residue(s) may be positioned at any one of positions b, c, e, f or g in one or more of the sequences (a b c d d′ e f g) in the α-helical peptide. In some embodiments, the lysine residue is positioned at position b, especially position b of the first sequence (a b c d d′ e f g) in the α-helical peptide of the polypeptide or protein.

In another embodiment, the at least one stimuli-responsive amino acid residue is an amino acid residue that has a side chain that can bind a metal ion. For example, the at least one stimuli-responsive amino acid residue may be a histidine residue or a residue containing a carboxylic acid in its side chain, such as glutamic acid or aspartic acid.

In a particular embodiment, the at least one stimuli-responsive amino acid residue is at least one histidine residue. In some embodiments, each α-helical peptide in the polypeptide or protein has one histidine residue. In other embodiments, each α-helical peptide in the polypeptide or protein has more than one histidine residue, for example, two, three or four histidine residues or one histidine residue per sequence (a b c d d′ e f g) in the a-helical peptide of the polypeptide or protein. The histidine residue may be present at any one of positions b, c, e, f or g in one or more of the sequences (a b c d d′ e f g) in the a-helical peptide. In some embodiments, the histidine residue is positioned at position b or f. In some embodiments, a histidine residue may be present at position b of one or more sequences (a b c d d′ e f g) or position f of one or more sequences (a b c d d′ e f g). In some embodiments, a histidine residue is present at position b of one or more of sequence (a b c d d′ e f g) and at position f in one or more other sequences (a b c d d′ e f g) in the a-helical peptide of the polypeptide or protein.

In another embodiment, the at least one stimuli-responsive amino acid residue results in each sequence (a b c d d′ e f g) in the α-helical peptide in the polypeptide or protein having a net positive or negative charge of 1 or 2 at a specified pH. In some embodiments each sequence in each α-helical peptide in the polypeptide or protein has a net positive charge. For example, when the sequence is at a specified pH, if an acidic amino acid residue is present it is protonated and therefore has no charge or if the acidic amino acid residue is charged the sequence contains more than one basic residue and the basic residues such as lysine, arginine or histidine have a positive charge providing a net positive charge. Alternatively, when the sequence is at a specified pH, any basic residue is not protonated and therefore has no charge or if the basic amino acid is charged, the sequence contain more than one acidic residue and the acidic residues are charged to provide a net negative charge. The pH required to provide the net negative or net positive charge will be determined by the pK₂ of the acidic and/or basic residue side chains in the sequence and the balance of positive versus negative residues, other than the stimuli-responsive residue, for example, the balance of arginine vs glutamic acid and/or aspartic acid residues when lysine is stimuli-responsive residue.

In some embodiments, the at least one stimuli-responsive amino acid residue is a charged amino acid residue. The charged amino acid residue may bear a positive or negative charge. Examples of suitable charged amino acid residues include lysine, arginine, histidine, ornithine, glutamic acid and aspartic acid.

In some embodiments, the stimulus alters the zeta potential and surface charge of the biosurfactant at the liquid-gas interface. In some embodiments, the zeta potential and surface charge are altered by altering the pH of the biosurfactant. In some embodiments, the stimulus is an acid. In other embodiments, the stimulus is a base. In yet other embodiments, the pH may be altered by dilution of the bulk aqueous phase from which the foam is formed. Altering the pH of the foam may alter the charge of the side chain substituents on the biosurfactant.

If two substituents in close proximity, either on the same biosurfactant molecule or on adjacent biosurfactant molecules bear the same charge at a given pH, the substituents will repel one another. This repulsion may lead to destabilization of a foam by disruption of intra- or intermolecular interactions. For example, biosurfactant comprising at least one lysine residue may be used to form a stable foam at a pH at which the lysine side chain amino groups are uncharged when at the liquid-gas interface, for example, at pH 8.5. Addition of acid to the bulk liquid phase sufficient to reduce the pH of the foam or removal of the bulk liquid phase and replacement with a second bulk liquid phase with a lower pH, results in a change in pH of the foam. Reduction of pH such that the pH near the liquid-gas interface is below the interfacial pK₂ of the lysine amino substituent will cause the amino group to become protonated, and positively charged. The introduction of one or more charges in a biosurfactant molecule may reduce interactions between biosurfactant molecules at the liquid-gas interface or destabilize interactions within a biosurfactant resulting in destabilization of the foam.

Addition of base or dilution of the bulk aqueous solution may alter the charge on the biosurfactant molecules to reduce or remove repulsion between the biosurfactant molecules resulting in the formation and maintenance of a stable foam. For example, addition of base to increase the pH above the pK₂ of the amino group of a lysine residue will result in the lysine residues in the biosurfactant becoming uncharged and therefore interactions between biosurfactants may increase thereby stabilizing the foam. Alternatively, the introduction of a charge in close proximity to a charge of opposite sign may strengthen interactions between biosurfactant molecules at the liquid-gas interface or stabilize interactions within a biosurfactant, for example, by introduction of a salt bridge, resulting in stabilization of the foam.

Alternatively, when two foam bubbles are at close proximity there are two liquid-gas interfaces at close proximity. If the biosurfactant layer at each liquid-gas interface lacks a net charge, the two interfaces will not repel one another and may approach and coalesce to form a larger bubble, ultimately resulting in the collapse of the foam. Conversely, if biosurfactant layers are exposed to a stimulus that alters the pH and therefore surface charge of the biosurfactant layers at each liquid-gas interface such that the net charge of the biosurfactant layer at the liquid-gas interface becomes charged, either a net positive charge or net negative charge, the two interfaces will repel one another thereby stabilizing a foam.

In addition, the ionization constant or pK₂ of an acidic or basic group may be altered as a result of adsorption at an air-water interface. The ionization constant or pK₂ of an acidic or basic group is dependent on factors such as proximity to neighbouring charges and the hydrophobicity and dielectric constant of the surrounding environment. It may also be affected by the ionic strength of the aqueous solution. For example, the pK₂ of amino acids can be tuned by 4-5 units by control of ionic strength in the subphase and through changes in the interfacial dielectric constant (Ariga et al., J. Am. Chem. Soc., 2005, 127, 12074-12080). In general, the liquid-gas interface has an excess of hydroxide ions and the ionization constants of acidic or basic groups at an air-water interface change in a direction that favours electrical neutrality at the interface. These effects generally lead to an increase in the pK₂ of acidic groups when adsorbed at the liquid-gas interface, and a decrease in the pK₂ of basic groups (Ariga et al., ibid).

Suitable acids and bases are those which are soluble in and alter the pH of the biosurfactant solution from which the foam is formed. The acids and bases may be inorganic or organic. Illustrative examples of suitable inorganic acids include, but are not limited to, hydrochloric acid, hydrofluoric acid, hydrobromic acid, hydroiodic acid, nitric acid, sulfuric acid and phosphoric acid. Illustrative examples of suitable organic acids include, but are not limited to, acetic acid, formic acid, propionic acid, butyric acid, benzoic acid, citric acid, tartaric acid, malic acid, maleic acid, hydroxymaleic acid, fumaric acid, lactic acid, mucic acid, gluconic acid, oxalic acid, phenylacetic acid, methanesulphonic acid, toluenesulphonic acid, benzenesulphonic acid, salicylic acid, sulphanilic acid, ascorbic acid and valeric acid, succinic acid, glutaric acid and adipic acid. Illustrative examples of suitable bases include but are not limited to ammonia, organic amines, sodium hydroxide, potassium hydroxide, calcium hydroxide, magnesium hydroxide, sodium carbonate, potassium carbonate, magnesium carbonate, calcium carbonate, sodium bicarbonate, potassium bicarbonate, magnesium bicarbonate and calcium bicarbonate:

In some embodiments, the stimulus alters metal ion binding of the biosurfactant. In some embodiments, the stimulus is a metal ion or a chelating agent. Suitable metal ions include any metal ions or combination of metal ions able to form bridges within a between different biosurfactant molecules. Illustrative examples of suitable metal ions include, but are not limited to, magnesium ions and calcium ions, transition metal ions such as titanium ions, vanadium ions, chromium ions, manganese ions, iron ions, cobalt ions, nickel ions, copper ions, zinc ions and molybdenum ions, and lanthanide ions such as lanthanum ions, cerium ions, praseodynium ions, neodynium ions, promethium ions, samarium ions, europium ions, gadolinium ions, terbium ions, dysprosium ions, holmium ions, erbium ions, thalium ions, ytterbium ions and lutetium ions.

In some embodiments, metal ions may form bridges between different biosurfactant molecules at the liquid-gas interface both in the plane of the interface and perpendicular to the interface thereby forming a structural architecture at the interface.

In some embodiments, the stimulus that alters metal ion binding of the biosurfactant alters the availability of metal ions in bulk solution for binding with the biosurfactant. For example, the addition of metal ion chelators will bind metal ions in the bulk solution and prevent metal ion bridges forming within or between biosurfactant molecules or may remove metal ions from metal ion bridges within or between biosurfactant molecules. A metal ion chelator may therefore weaken the interactions between biosurfactant molecules by destabilizing biosurfactant conformation and/or reducing interactions between biosurfactant. The metal ion chelators may scavenge adventitious metal ions present in the bulk solution or dispersion from which the foam is formed. Alternatively, the metal ion chelators may scavenge metal ions that have been previously added to strengthen the interactions within or between the biosurfactant molecules. Suitable chelating agents are those which are soluble in the bulk biosurfactant solution from which the foam is formed and/or may be selected for suitability or ability to bind a particular metal ion. For example, suitable chelating agents include, but are not limited to, ethylenediamine, ethylenetriamine, triethylenetetramine, ethylenediaminetetraacetic acid (EDTA), aminoethanolamine, ethylene glycol bis(2-aminoethyl ether)-N,N,N′N′-tetraacetic acid (EGTA), tris(2-imidazolyl)carbinol, tris[4(5)-imidazolyl]carbinol, bis[4(5)-imidazolyl]glycolic acid, oxaloacetic acid, citric acid, glycine or other amino acids, salicylate, macrocyclic ethers, multidentate Schiff bases, acetylacetone, bis(acetylacetone) ethylenediimine, 2-nitroso-1-naphthol, 3-methoxyl-2-nitrosophenol, cyclohexanetrione trioxime, diethylenetriaminepentaacetic acid (DTPA), N-(hydroxyethyl)ethylenediaminetriacetic acid (HEDTA), tripolyphosphate ion, nitrilotriacetic acid, dimethylglyoxime, dimercaprol, deferoxamine.

The amount of metal ion required may be determined by simple screening techniques, where a sample of the bulk solution is subjected to foam forming conditions in the presence of different amounts of metal ion or chelating agent. When considering amounts of metal ion or chelating agent to use, the pH range at which the chelating agent provides effective chelation of the metal ion present should be considered.

The stimulus that alters metal ion binding by reducing metal ion availability may be an ion with which the metal ion forms an insoluble salt thereby removing the metal ion from the bulk liquid phase. Suitable ions include, but are not limited to, phosphate ions, borate ions, sulfide ions, arsenate ions, carbonate ions and chloride ions. In addition, in some cases a metal ion may be precipitated as a hydroxide by an increase in pH. The effect is removal of the metal ion by precipitation.

In another embodiment, the stimulus that alters metal ion binding by reducing metal ion availability may be a metal ion adsorbent such as a zeolite. In a particular embodiment, the zeolite is a salt of aluminium silicate such as sodium aluminium silicate (zeolite A). In some embodiments the zeolite is combined with a polycarboxylic acid such as polyacrylate.

In some embodiments the stimulus that alters metal ion binding by reducing metal ion availability may be a monodentate ligand that supplements metal ion binding within a biosurfactant and stabilizes a foam or replaces an interaction between a biosurfactant and a metal ion destabilizing the foam.

In an alternative embodiment, the binding of a metal ion may give rise to a local positive charge which could interact with a nearby positive charge, or may neutralize a negative charge which previously stabilized an ordered conformation, or may cause the average positive charge on a biosurfactant to deviate significantly from zero generating charge-charge repulsions. In these cases, the binding of a metal ion may destabilize the foam causing weakening or collapse of the foam and the addition of a chelating agent may stabilize the foam by scavenging adventitious metal ions that may cause destabilization.

In a particular embodiment, the α-helical peptide in the polypeptide or protein comprises at least one histidine residue. Histidine residues contain an imidazolyl group which is able to bind metal ions. The addition of metal ions, particularly Zn⁺⁺ and Ni⁺⁺ ions, results in a metal ion bridge between two histidine residues in close proximity at the liquid-gas interface and strengthening of interactions between biosurfactant molecules or between a-helices within a biosurfactant molecule thereby stabilizing any foam formed. The addition of a chelating agent, precipitating agent, monodentate ligand or adsorbant that removes the metal ions, or a change in pH sufficient to neutralize the charge on histidine thereby removing metal ion binding potential of histidine, disrupts these interactions and destabilizes the foam.

Surprisingly, it has been found that foam stability can also be modulated using salts to increase or decrease hydration around a biosurfactant layer at the liquid-gas interface. In some embodiments, the stimulus alters the hydration structure of the biosurfactant. The biosurfactant molecules are amphipathic molecules having a hydrophobic portion and a hydrophilic portion. While in bulk aqueous solution the biosurfactant folds to provide a tertiary structure that has a substantially hydrophilic surface and a substantially hydrophobic core, the distinct hydrophilic and hydrophobic areas of the biosurfactant molecule also provide it with amphipathic properties and an affinity for the liquid-gas interface. It is known that the tertiary folding of the polypeptide is lost on adsorption at the gas-liquid interface allowing the hydrophobic portion of the molecule to orientate itself to the gas phase and the hydrophilic portion to extend into the liquid phase. The hydrophilic portion of the biosurfactant is also hydrated by water molecules from the bulk aqueous phase. Without wishing to be bound by theory, when a biosurfactant film at a gas-liquid interface of a foam is well hydrated, in such a way that the water molecules are oriented, net repulsive forces can occur. Two biosurfactant films in the foam are then not able to approach each other and interact in a way that would cause collapse of the foam. If the biosurfactant molecules at an interface lack structured water of hydration or have reduced structured water, two biosurfactant films within a foam may approach each other and interact resulting in destabilization or collapse of the foam.

In some embodiments, the stimulus that alters the hydration structure of the biosurfactant at the liquid-gas interface is a kosmotropic salt. In other embodiments, the stimulus that alters the hydration structure of the biosurfactant molecule at the liquid-gas interface is a chaotropic salt.

When the stimulus is a kosmotropic salt, the kosmotropic ions are also well hydrated and interact with and add to the hydration of the biosurfactant molecules and/or cause enhanced ordering of the hydration layer. The kosmotropic ions prevent two biosurfactant films at adjacent liquid-gas interfaces in the foam approaching one another and interacting and thereby stabilize the foam. In contrast, when the stimulus is a chaotropic salt, the chaotropic ions decrease or disrupt the orientation and/or extent of structured water from the hydration layer of a biosurfactant film at an interface. The addition of chaotropic salts therefore allows two biosurfactant films at adjacent liquid-gas interfaces in the foam to approach one another and interact thereby destabilizing the foam and resulting in the collapse of the foam.

Suitable stimuli that are kosmotropic salts include sulphates, fluorides, carbonates, magnesium salts, lithium salts and calcium salts. In a particular embodiment, the kosmotropic salt is a sulphate salt, especially ammonium sulphate, sodium sulphate, potassium sulphate, calcium chloride and lithium chloride.

The stimulus may be a chaotropic salt. Suitable chaotropic salts include guanidinium salts, thiocyanate salts, perchlorate salts and iodide salts. In some embodiments the chaotropic salt is guanidinium chloride, guanidinium thiocyanate or sodium thiocyanate.

A suitable amount of kosmotropic or chaotropic salt may be determined by simple screening methods where foam formation is performed in the presence of varying amounts of kosmotropic or chaotropic salt and stabilization or destabilization observed.

In some embodiments, the amount of kosmotropic or chaotropic salt is in the range of 100 mM to 2 M, especially 200 mM to 1 M, more especially 400 mM to 700 mM, for example about 500 mM.

In some embodiments, the foam is stabilized. In other embodiments, the foam is destabilized. In some embodiments, the foam is stabilized at a first time and destabilized at a second time. In some embodiments, the modulation includes multiple stabilization and destabilization steps.

Accordingly, the method of modulating the stability of a foam may further comprise the step of
ii) exposing the biosurfactant to a second stimulus that alters the pH of the foam or the metal binding of the biosurfactant or hydration structure of the biosurfactant at the liquid-gas interface adopted on exposure to the stimulus in step i).

In some embodiments, the first stimulus in step i) stabilizes the foam and the second stimulus destabilizes the foam. In other embodiments, the stimulus in step i) destabilizes the foam and the second stimulus stabilizes the foam.

In some embodiments, steps i) and/or ii) are repeated one or more times.

This allows the foam to be formed in a controlled manner, maintained for a desired period of time and collapsed at a desired time and optionally reformed and collapsed at subsequent times, optionally multiple times.

In a particular embodiment, there is provided a method of modulating the stability of a foam comprising the steps of:
i) forming a stable foam from a foaming composition; said foaming composition comprising:
   a. a first bulk aqueous phase having a pH of 8 or above;
   b. a biosurfactant having at least two α-helical peptides linked by a linking sequence of 3 to 11 amino acid residues, wherein each α-helical peptide comprises a sequence of amino acid residues:
      (a b c d d′ e f g)ₙ
   wherein n is an integer from 2 to 12;
   amino acid residues a and d are hydrophobic amino acid residues;
   amino acid residue d′ is absent or is a hydrophobic amino acid residue;
   at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
   amino acid residue g is any amino acid residue;
   wherein each α-helical peptide comprises a lysine residue;
ii) removing the first bulk aqueous phase from the foaming composition; and
iii) replacing the first bulk aqueous phase with a second bulk aqueous phase having a pH below 8.

In some embodiments, the first bulk aqueous phase has a pH of about 8.3 to 9.0, for example, 8.5. In some embodiments, the second bulk aqueous phase has a pH of less than 8, for example, 7 to 7.5. In some embodiments, the foam composition further comprises an antimicrobial peptide and/or an enzyme such as a protease, amylase, lipase or cellulase enzyme. This embodiment is particularly useful in controlling foam stability during a laundry wash cycle and foam collapse at the beginning of a laundry rinse cycle or controlling foam stability during hand washing and collapse of the foam at the beginning of rinsing during a low rinse hand wash cycle.

In some embodiments, the foam composition further comprises an α-helical peptide of the invention, particularly an α-helical peptide that comprises a lysine residue.

The foam comprising the biosurfactant may be prepared by dissolving or dispersing the biosurfactant in a liquid to form a solution or dispersion and mixing the solution or dispersion with a gas, or by simple agitation, as in a washing machine, where air becomes incorporated into the aqueous phase due to the agitation.

The mixing may be any means of mixing liquid and gas known in the art to form foams. In some embodiments, the gas is bubbled through the liquid phase. In other embodiments, the liquid is mixed or agitated in the presence of gas. The vigorousness of agitation or mixing or the rate of flow of gas into and through or into the liquid will determine the speed with which the foam forms and the size of the bubbles in the dispersed gas phase as is known in the art of foam formation.

In some embodiments, the liquid phase is a polar liquid which is capable of dissolving the biosurfactant to form a solution. Alternatively, the biosurfactant is insoluble or only partially soluble in the polar liquid and a dispersion is formed. In other embodiments, the liquid phase is a non-polar liquid capable of dissolving the biosurfactant to form a solution. Alternatively, the biosurfactant is insoluble or only partially soluble in the non-polar liquid and a dispersion is formed. Examples of suitable polar liquids include, but are not limited to, water, buffers, methanol, ethanol, isopropanol, acetonitrile or mixtures thereof. Examples of suitable non-polar liquids include, but are not limited to, hydrocarbons such as pentane, hexane, octane and mixtures of hydrocarbons, liquid oils such as olive oil, sunflower oil, safflower oil, grapeseed oil, sesame oil, coconut oil, canola oil, corn oil, flaxseed oil, palm oil, palm kernel oil, peanut oil and soybean oil or triacylglycerols which are rich in unsaturated fatty acids or mixtures thereof. The gas may be any gas suitable for the application for which the foam is used. Suitable gases include, but are not limited to, air, nitrogen, oxygen, hydrogen, helium and argon. In a particular embodiment, the gas phase is air and the liquid phase is water.

### Applications

The biosurfactant proteins and polypeptides of the present invention may be used in the controlled formation and collapse of foams used in foods, beverages, pharmaceuticals, personal care products, low-rise medical foams, cosmetics, cleaning products, inks and printing, surfactants, waste water treatment, explosives, mineral recovery, bioremediation, corrosion inhibition, petrochemicals, oil recovery, dental care and biotechnology.

The present invention may be useful in the control of foaming in cleaning products such as laundry detergents. The biosurfactant proteins and polypeptides of the invention may be incorporated into laundry detergents to provide stable foam for the wash cycle, which occurs at alkaline pH, typically above pH 8.5. When the wash water is drained and replaced with rinse water, the pH of the bulk liquid phase is reduced, for example, below pH 8 and the foam is destabilized and collapses. The biosurfactant is then readily removed during the rinse cycle. A biosurfactant protein or polypeptide in which each α-helical peptide comprises a lysine is particularly useful in this application. The biosurfactant protein or polypeptide may be produced as a fusion protein with an antimicrobial peptide and after cleavage of the fusion protein, used as a composition comprising both biosurfactant and antimicrobial peptide. Alternatively the biosurfactant protein or polypeptide may be produced as a fusion protein with an enzyme, such as a protease, lipase, amylase or cellulase enzyme, and optionally another protein carrier, and after cleavage of the fusion protein and optionally removal of the carrier protein, used as a composition comprising both biosurfactant and enzyme.

The present invention may also be used to prepare compositions comprising a biosurfactant and an antimicrobial peptide, such as PAC-113 and MBI-266, that may be useful in pharmaceutical compositions. The pharmaceutical compositions may be topical compositions applied to the skin or mucous membranes such as those in the mouth, throat, nose, vagina and anus. For example, the composition may be in the form of a low-rinse antimicrobial hand foam useful in personal care or clinical settings, or in barrier protection for burns victims or where skin is infected. In some embodiments, the biosurfactant proteins and polypeptides of the invention may be incorporated into low-rinse wash compositions to provide stable foam during washing, which occurs at alkaline pH, typically above pH 8.5. When rinsing occurs, the pH of the bulk liquid phase is reduced, for example, below pH 8 and the foam is destabilized and collapses. The biosurfactant is then readily removed during the rinse cycle. A biosurfactant protein or polypeptide in which each α-helical peptide comprises a lysine is particularly useful in this application.

The invention may be useful in a plurality of applications where it is desirable that the properties of a foam respond to contact with the human body, for example by responding to a change in pH, or the presence of metal ions or certain salts. For example, it may be desirable to alter the stability of a food or beverage foam on exposure to the pH and temperature characteristic of the human mouth, altering the flavour release properties, mouthfeel, viscosity or other properties of the foam. Alternatively, it may be desirable to alter the stability of a dental foam on exposure to the pH and temperature characteristic of the human mouth or the mouth of a particular non-human species, for example to transform a stable and less active stored form of a dental care product into a more active form. Alternatively, it may be desirable to alter the stability of a personal care or cosmetic foam on exposure to the temperature and pH characteristic of human skin, for example to enhance the appearance of a cosmetic or personal care product. In another example, it may be desirable to alter the stability of a pharmaceutical foam on exposure to the temperature and pH characteristic of human skin, for example to enhance skin permeation by a pharmaceutical product. Advantageously, these products may also comprise an antimicrobial peptide.

The invention may further be useful in a plurality of applications in which it is desirable to employ foam for the recovery or purification of a desired material by flotation. The invention may further be useful in a plurality of applications in which it is desirable to employ foam for the removal of an undesired material, such as a waste material or contaminant, by flotation. In these cases, the use of foam for flotation recovery or purification of a desired material may be followed by breaking of the foam for convenient further applications of the desired material. Alternatively, the use of foam for flotation removal of an undesired material, such as a waste material or contaminant, may be followed by breaking of the foam for convenient further disposal of the undesired material.

The invention may be useful in a plurality of applications where it is desirable to control the wetting or coating of a surface. In these cases a biosurfactant-containing foam, solution, or dispersion is provided which has particular properties of wetting or coating a surface under a first set of conditions, and distinct properties of wetting or coating a surface under a second set of conditions. This may be useful either in controlling the wetting of an entire surface, in the generation of desired patterns on a surface. Alternately, such controllable wetting may be useful in sensor applications, or in imaging.

The proteins and polypeptides of the present invention may also be used as intermediates in the preparation of smaller peptide surfactants. In this case, after preparation of the protein and polypeptide, the cleavable linker linking the α-helical peptides is cleaved to provide at least two peptides. The peptides may then be used in other applications including as peptide surfactants.

The proteins and polypeptides of the present invention may also be used to enhance the manufacture of other peptides, polypeptides or proteins using recombinant DNA technology. The proteins and polypeptides of the present invention are expressed at high levels in fermentation and their use as a carrier in a fusion protein with another peptide, polypeptide or protein of interest can enhance the expression of the peptide, polypeptide or protein of interest. The fusion protein can then be cleaved and the protein, polypeptide or peptide can optionally be isolated from the biosurfactant. Examples of proteins, polypeptides or peptides that may be prepared by this method include antimicrobial peptides, antibodies, enzymes useful in laundry detergents such as protease, amylases, lipases and cellulases; and peptides useful in the manufacture of metallic or semiconductor nanostructures.

In order that the invention may be readily understood and put into practical effect, particular embodiments will now be described by way of the following non-limiting examples.

### EXAMPLES

### Example 1

### Preparation of Biosurfactant Polypeptides or Proteins

### General Method

Chemically competent E. coli BL21(DE3) cells are transformed with the engineered pET48b expression plasmid using the heat-shock transformation method, and then stored as glycerol stocks. From these stocks, LB plates (Amresco LB agar, Miller formulation, tissue culture grade, Solon, Ohio) containing 15 μg mL⁻¹ kanamycin sulphate (Gibco, Invitrogen, SKU#11815) are streaked and a single colony selected for expression.

Expression may be achieved using shake flask cultures or in a fermenter as set out below.

Shake flask cultures prepared as follows:

### Method Overview

For all constructs, a starter culture was grown from a single colony picked from freshly streaked glycerol stock plates (LB agar-KanS 15 μg/mL). This starter culture was used to inoculate 1000 mL of LB Kan 15 μg/mL in shake flask cultures. The cultures were incubated at 37° C. until the OD₆₀₀ reached 0.5, at this point each culture was induced with 1 mM isopropyl-β-D-1-thiogalactopyranoside (IPTG) and further incubated at 26° C. overnight until harvest (16-18 hrs). 1 mL samples were taken at 0 hr and O/N (16-18 hrs) time points. The samples were lysed in Bugbuster (Novagen) to analyze for expression in the total and soluble fractions. The samples were analyzed by SDS-PAGE.

In some cases the temperature was not decreased at the point of induction, and the culture medium was varied:

### Method Overview

The expression constructs, pET48—SEQ ID NO:6 and SEQ ID NO:7 were transformed into BL21 de3 expression strain. Small-scale expression cultures were setup in 100 mL LB (Kan 15 μg/mL). The cultures were induced when the OD₆₀₀ reached between 0.5 and 0.7 and expression continued at 37° C. 1 mL samples were taken for analysis (via SDS-PAGE) at the 0 hr, 2 hr and 4 hr time points. The samples were lysed in Bugbuster (Novagen) to analyze expression in the total, soluble and insoluble fractions. The samples were analyzed by SDS-PAGE.

The parameters in each case can vary without any major effect on expression level. For example, a slightly lower solubility is achieved for some sequences when cultivated at 37° C. instead of 26° C., however, the cultivation is for a reduced time (4 hr instead of 16 hr post induction). In some cases a trade off of solubility vs expression time may be achieved at 32° C.

For SEQ ID NO:1, a fermenter was also used:

E. coli BL21 de3 pET-48b(+)-SEQ ID NO:1 was inoculated from a shake flask into 3.0 L modified C1 minimal media (Middelberg et al., Biotechnology and Bioengineering, 1991, 38, 363-370) in a 7-L glass fermenter (Applikon). The pH was controlled at 7.1 by the automatic addition of 14% (v/v) NH₄OH. Foaming was controlled by periodic manual injection of Antifoam-C(Sigma): Dissolved oxygen was controlled at a minimum DO set-point of 25% of air saturation at 37° C., by stirrer speed and oxygen supplementation of sparged air. When the carbon source was exhausted, a step-wise linear feed (250 g/L glucose, 50 g/L (NH₄)₂SO₄, 5 g/L MgSO₄) was implemented a rate of 65 g/h (0-4.5 h), 90 g/h (4.5-13.5 h) and 115 g/h (13.5-17.5 h). The temperature was controlled at 37° C. during the growth phase and lowered to 26° C. just prior to induction with 1 mM IPTG, which occurred 2 h after the linear feed commenced. The fermentation was terminated 15.5 h after induction by switching off the nutrient feed and air flow, and reducing the temperature set point to 14° C.

Cell pellets may be obtained from any of the culture methods by centrifugation, for example, Beckman Coulter-Avanti J-20 XPI for 15 min at 9000×g and 4° C. The pellets may be stored at −80° C. until further use.

The following polypeptide biosurfactants were obtained by the above methods:

| | |
|---|---|
| SEQ ID NO: 1: | MD(PS-MKQLADS-LHQLARQ-VSRLEHA-D)₄ |
| SEQ ID NO: 2: | MD(PS-MKQLADS-LHQLARQ-VSRLEHA-D)₂ |
| SEQ ID NO: 3: | MD(PS-AKSLAES-LHSLARS-VSRLEHA-D)₄ |
| SEQ ID NO: 4: | MD(PS-AKSVAES-LHSLARS-VSRLVEHA-D)₄ |
| SEQ ID NO: 5: | MD(PS-AHSVAES-LHSLARS-VSRLVEHA-D)₄ |
| SEQ ID NO: 6: | MD(PS-AHSVAKS-LHSLARS-VSRLVSHA-D)₄ |
| SEQ ID NO: 7: | MD(PS-AHSVAES-LHSLAES-VSELVSHA-D)₄ |
| SEQ ID NO: 8: | MD(PS-AQSVAQS-LAQLAQS-VSQLVSQA-D)₄ |
| SEQ ID NO: 9: | MD(PS-ANSVANS-LANLANS-VSNLVSNA-D)₄ |
| SEQ ID NO: 10 | MD(PS-AQSVAES-LAQLAES-VSELVSQA-D)₄ |
| SEQ ID NO: 11 | MD(PS-ANSVAES-LANLAES-VSELVSNA-D)₄ |

### Example 2

The small-scale shake flask method of Example 1 was repeated to produce a peptide analogous to SEQ ID NO:1 in which the linker sequence between the α-helices was only two residues, DP. No expression of the polypeptide was observed.

### Example 3

### Preparation of Cell Disruptates

Cell disruptates may be prepared directly from the fermentation broth or from frozen cell-suspensions prepared from the fermentation broth. If a frozen cell suspension was used, the cell suspension was thawed before use and re-suspended in an appropriate buffer or water.

Sonication was used for cell disruption, using a “Sonifier 450” from Branson, with ultrasonic waves of a frequency of 20 kHz.

The cells were sonicated twice for 1 minute. Much of the energy, absorbed by the cell suspension, was converted to heat. Thus effective cooling is essential during sonication.

For the analysis of expression levels only, BugBuster was used to chemically disrupt cells and allow product release and analysis of supernatant and pellet samples following small-scale centrifugation.

Centrifugation was used in some cases for clarification of E. coli cells or disruptates, using a microfuge (Sorvall® Biofuge primo R). Samples were centrifuged at 15,000 rpm for 5 minutes to separate the insoluble from the soluble fraction thus give a clarified composition. Insoluble components include cells, cell-debris, associated denatured proteins, DNA or RNA and protein-aggregates.

### Example 4

### Ammonium Sulphate (AS) Precipitation

A cell disruptate was prepared by sonication and the OD₆₀₀ measurement of E. coli cells before disruption was determined by UV/VIS spectroscopy.

Harvested cells (15,000×g, 5 min) were re-suspended with HEPES buffer (25 mM HEPES, 20 mM NaCl, pH 7.6) to an OD₆₀₀ of 15, followed by sonication to break the cells. Disruptate containing cell debris was chosen for the precipitation experiments, as centrifugation prior to AS addition had no significant impact on yield nor purity. Experiments were performed in small scale volumes of 1 mL at varying pH values and at different AS concentrations. AS addition occurred by a prior prepared 4.0 M AS solution to adjust the final concentrations and volumes, before adjusting the pH by titration with a 1.0 M HCl solution. After one hour of incubation, the samples were centrifuged to separate the insoluble aggregate fraction from still soluble proteins in solution. The supernatant was analyzed by SDS-PAGE to determine qualitative SEQ ID NO:1 and protein amounts in solution. The insoluble pellets of precipitates were re-dissolved in HEPES buffer to the same volume as the initial reaction mixture and also analyzed by SDS-PAGE to determine the purity of SEQ ID NO:1 aggregates.

Initial experiments were performed with different AS concentrations from 0.5 M to 2.0 M, while varying the pH from pH 5 to pH 8, to determine if lower pH values causes better precipitation of impurities, due to a probable combinatorial isoelectric precipitation effect and enhanced contaminant unfolding.

Generally it was observed that higher AS concentrations caused a higher salting-out of proteins and thus a higher purity of the solution. SEQ ID NO:1 could be kept almost completely in solution at AS concentrations between 0.5 M and 1.5 M, while at 2.0 M higher amounts of SEQ ID NO:1 co-precipitated.

At pH values between 5 and 9 and at an AS concentration of 1.5 M a high ratio of impurities could be precipitated by keeping SEQ ID NO:1 with high yield in solution, as shown by SDS-PAGE analysis.

By performing the experiments at pH values of 2 to 4, a noticeably better purity was achieved, by keeping SEQ ID NO:1 mainly in solution. From SDS-PAGE analysis it was observed that at a pH of 4 more SEQ ID NO:1 stayed in solution than at pH values of 2 and 3, but the SEQ ID NO:1 loss seemed to be slightly higher than at pH values of 5 and above, yet with improved purity.

These experiments were performed at room temperature. Further efforts were made to optimize the purity at neutral pH, by performing the experiments under cooled conditions, by enhancing the incubation time and by raising the AS concentration. However, none of these conditions resulted in an obvious enhancement of the purity of SEQ ID NO:1.

### Example 5

### Ammonium Sulphate Precipitation of Cell Disruptates

Further experiments were performed as set out in Example 4 at pH 3, pH 4 and pH 7 at AS concentrations of 1.5 M, to compare the purity and yield of obtained SEQ ID NO:1 at different pH values. Also different AS concentrations of 0 M, 0.5 M, 1.0 M and 1.5 M at a pH of 4 were chosen to compare purity and yield at different AS concentrations.

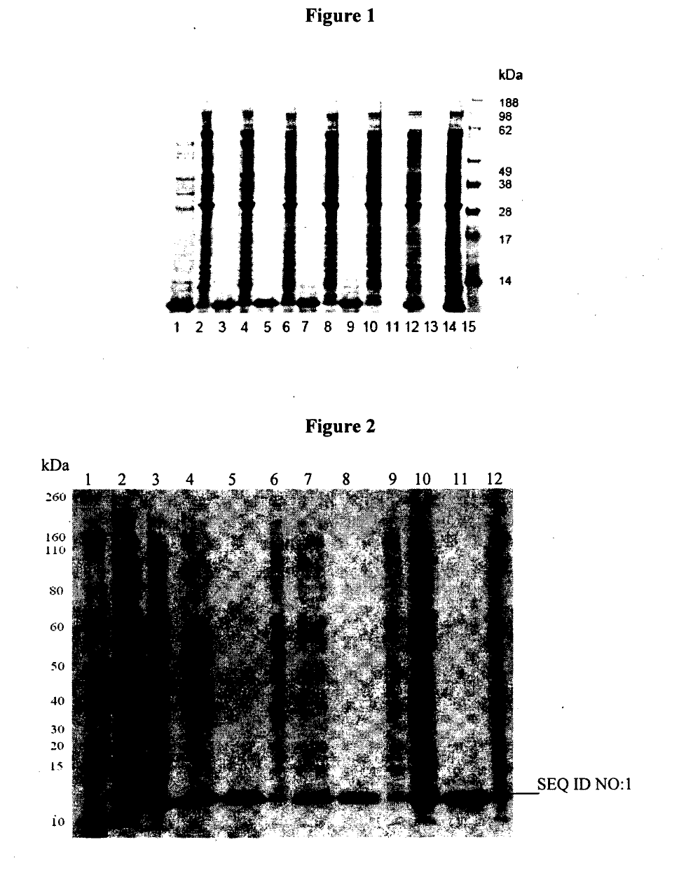

Results are shown by SDS-PAGE gel in FIG. 1. Samples were also analyzed by RP-HPLC to obtain quantitative data for SEQ ID NO:1 and to determine impurities.

From SDS-PAGE analysis (FIG. 1) and RP-HPLC analysis it was shown that precipitation conditions of pH 4 and 1.5 M AS (Lane 5=supernatant (SN), Lane 6=pellet) resulted in better purity than lower AS concentrations of 0.5 M (Lane 9, SN) and 1.0 M (Lane 7, SN).

Without the presence of AS (lane 11, SN, Lane 12, pellet) at pH 4, all proteins and SEQ ID NO:1 precipitated completely, while raising AS concentration up to 1.0 M at pH 4 caused a stronger salting-in of SEQ ID NO:1 and very few cell proteins.

Furthermore, it was observed that precipitation at 1.5 M AS at pH 3 (Lane 5, SN) resulted in slightly better purity than pH 4 (Lane 3, SN) but also in a higher SEQ ID NO:1 loss.

At pH 7 (Lane 1, SN and 1.5 M AS) the highest amount of SEQ ID NO:1 stayed in solution, but purity was considerably lower compared to pH 3 (Lane 5, SN) and pH 4 (Lane 3, SN).

### Example 6

### SEQ ID NO:1 Purification Using Single-Step Thermal Treatment of Intact Cells

SEQ ID NO:1 protein was expressed within the cytoplasm of Escherichia coli using shake flasks and complex (LB) medium as described in Example 1. FIG. 2 provides an analysis of cell protein composition before (Lane 2) and after (Lane 3) the induction of protein expression. SEQ ID NO:1 protein accumulates to high levels within the recombinant cells.

Following expression the cell broth was divided into 3 samples which were used immediately without frozen storage: cell broth that was not treated further (sample 1); broth was centrifuged and cells were re-suspended in water to the same volume as the initial broth (sample 2), and; broth was centrifuged and cells were re-suspended in water to ⅕^{th} of the initial broth volume (sample 3), to test the effect of cell concentration.

### Sample 1 (Lanes 4, 5 and 6 in FIG. 2)

AS was added to fermentation broth to give a final concentration of 1.5 M. The suspension was heated at 90° C. for 20 min. Reaction mixture was centrifuged (15000×g, 10 min); supernatant was collected and precipitate was re-suspended in 500 μL PBS.

### Sample 2 (Lanes 7, 8 and 9 in FIG. 2)

Fermentation broth with expressed SEQ ID NO:1 protein (500 μL) was centrifuged (10000×g, 5 min), collected cells were re-suspended in 500 μL Milli-Q water and AS was added to a final concentration of 1.5M. The suspension was heated at 90° C. for 20 min. Reaction mixture was centrifuged (15000×g, 10 min); supernatant was collected and precipitate was re-suspended in 500 μL PBS.

### Sample 3 (Lanes 10, 11 and 12 in FIG. 2)

Fermentation broth with expressed SEQ ID NO:1 protein (2500 μL) was centrifuged (10000×g, 5 min), collected cells were re-suspended in 500 μL Milli-Q water and AS was added to a final concentration of 1.5M. The suspension was heated at 90° C. for 20 min. Reaction mixture was centrifuged (15000×g, 10 min); supernatant was collected and precipitate was re-suspended in 500 μL PBS.

As can be seen from FIG. 2, the SEQ ID NO:1 protein was obtained in high purity from the supernatant of each sample treated with ammonium sulphate at 90° C. (lanes 5, 8 and 11).

### Example 7

### SEQ ID NO:7 Purification Using Single-Step Thermal Treatment of Intact Cells in Deionized Water (A) and Culture Medium (B)

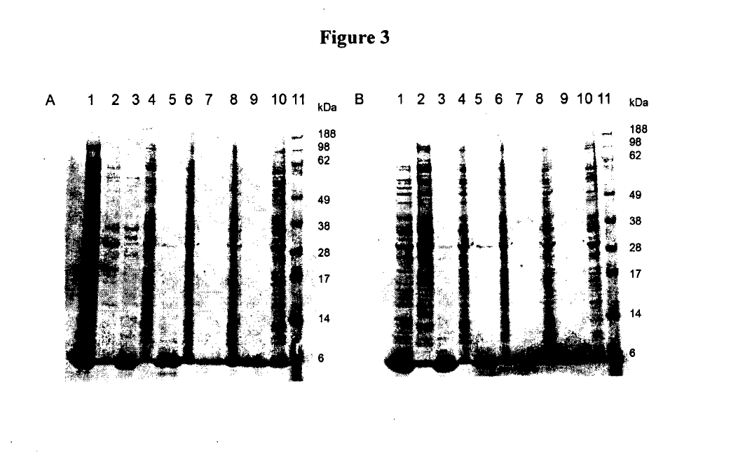

SEQ ID NO:7 (10.4 kDa) was expressed in Escherichia coli by shake flask cultures, using complex medium (LB agar-Kan). Expression involved cultivation at 37° C. and induction for 2 h with IPTG at this temperature. Cells were harvested by centrifugation and re-suspended either in Milli-Q water (A) or in fresh culture medium (B) to an OD₆₀₀ₙₘ of 20. FIG. 3 provides an SDS-PAGE analysis of the soluble (lane 1) and insoluble (lane 2) fractions of control samples (sonicated cells following re-suspension and sonication). Soluble SEQ ID NO:7 can be observed. FIG. 3A refers to experiments performed in Milli-Q water and Figure B refers to equivalent experiments performed in culture medium.

Three samples each of 500 μL cell suspension were used to test the effect of thermal treatment in the presence of ammonium sulphate (AS) (sample 1=0 M AS, sample 2=0.5 M AS, sample 3=1.5 M AS). Another sample of 500 μL (sample 4) was sonicated to break cells to test thermal treatment on broken cells following addition of AS to 1.5 M AS. In each case, treated material was centrifuged (15000×g, 5 min) to separate the soluble and insoluble fractions. Supernatant was collected and the pellet was resuspended in the same volume as the initial sample prior to treatment.

### Sample 1 (Lanes 3, 4 in FIGS. 3A and B)

Cell suspension with expressed SEQ ID NO:7 (500 μL) was diluted with Milli-Q water to adjust the final volume to 1 mL. The suspension was heated at 90° C. for 20 minutes. The reaction mixture was centrifuged (15000×g, 5 min); supernatant was collected and precipitate was resuspended in 1 mL PBS.

### Sample 2 (Lanes 5, 6 in FIGS. 3A and B)

AS was added to the provided cell suspension to give a final concentration of 0.5 M with a final volume of 1 mL. The suspension was heated at 90° C. for 20 minutes. The reaction mixture was centrifuged (15000×g, 5 min); supernatant was collected and precipitate was resuspended in 1 mL PBS.

### Sample 3 (Lanes 7, 8 in FIGS. 3A and B)

AS was added to the cell suspension to give a final concentration of 1.5 M with a final volume of 1 mL. The suspension was heated at 90° C. for 20 minutes. The reaction mixture was centrifuged (15000×g, 5 min); supernatant was collected and precipitate was resuspended in 1 mL PBS.

### Sample 4 (Lanes 9, 10 in FIGS. 3A and B)

AS was added to sonicated cells to give a final concentration of 1.5 M with a final volume of 1 mL. The suspension was heated at 90° C. for 20 minutes. The reaction mixture was centrifuged (15000×g, 5 min); supernatant was collected and precipitate was resuspended in 1 mL PBS.

SEQ ID NO:7 was observed in the supernatant fractions of all samples.

Generally it could be observed that experiments performed in culture medium showed better purity than those performed in Milli-Q water, probably due to an additional salting-out of contaminant host-cell proteins by culture medium-containing salts. While in the absence of AS during heating, still a few impurities were observed in solution, high amounts of those remaining impurities could be precipitated by the presence of AS. Higher AS concentrations caused a higher SEQ ID NO:7 loss, but also higher product purities.

### Example 8

### Isolation of SEQ ID NO:1

After removal of cell contaminants, the isolated supernatant containing SEQ ID NO:1 protein was further treated to precipitate the SEQ ID NO:1 protein. Three options were tried:

### 3.5 M AS Precipitation

During the first precipitation strategy for SEQ ID NO:1, after successful separation of most proteins, the AS concentration in the supernatant was raised to 3.5 M. At this concentration SEQ ID NO:1 was completely precipitated.

Experiments were performed by taking the SEQ ID NO:1 containing supernatant after contaminant precipitation and removal, and by adding appropriate amounts of solid AS. The experiments were performed at different pH values to determine any different effects on SEQ ID NO:1 precipitation. Besides, also an AS enhancement to 3.5 M by evaporation of water from the solution by dry inert nitrogen could be successfully tested for SEQ ID NO:1 precipitation. Thereby all pH values of pH 3 to 7 resulted in a total SEQ ID NO:1 precipitation at 3.5 M AS. Therefore no pH adjustment was required after the precipitation of impurities.

### pH 7.6, 5 mM Zinc Sulphate Precipitation

As an alternative to the 3.5 M AS precipitation, SEQ ID NO:1 was successfully precipitated by metal ion precipitation using 5 mM zinc in the form of ZnSO₄. A pH adjustment to pH of 7.4 to 7.8 and dilution of the AS containing supernatant were required to reduce the AS concentration to approximately 0.1 M to 0.2 M. In the presence of higher AS concentrations, SEQ ID NO:1 could stay stable in solution, due to salting-in by AS.

For a 1.5 M AS containing solution, a dilution of a factor of 8 to 10 had to be performed. Thus it was chosen to take the 1.0 M and 0.5 M supernatant after first-step precipitation, containing a few more impurities. For the 1.0 M solution a dilution of 5 was required and for the 0.5 M a dilution of 3 was enough to precipitate SEQ ID NO:1 almost completely at appropriate pH.

### Acid Precipitation

Another means for SEQ ID NO:1 precipitation was reducing the AS concentration to concentrations about 0.1 M to avoid a salting-in effect of SEQ ID NO:1 by AS and thus cause an acid precipitation of SEQ ID NO:1 at pH 3 or pH 4.

High dilutions were necessary, thus the 0.5 M supernatant was the best choice for this precipitation strategy, requiring dilutions of factor 5. In this case an almost complete SEQ ID NO:1 precipitation could be effected.

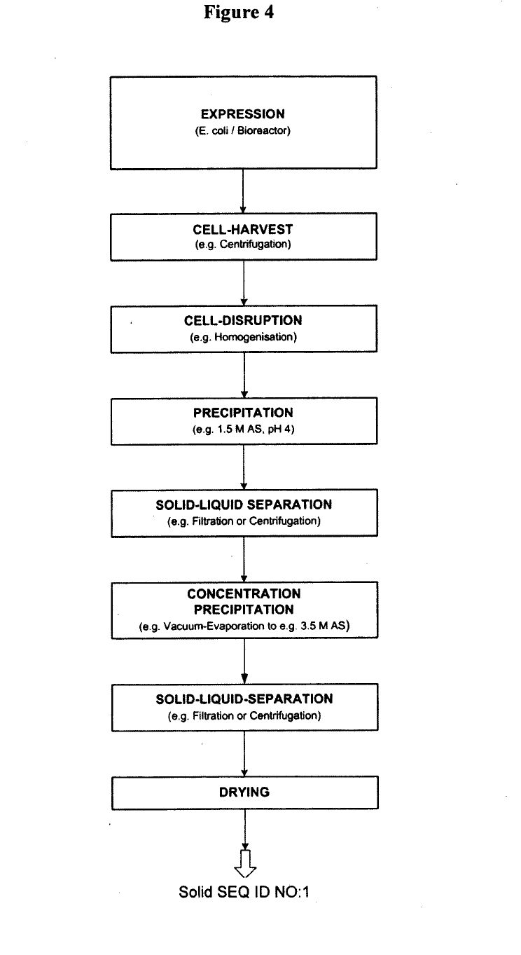

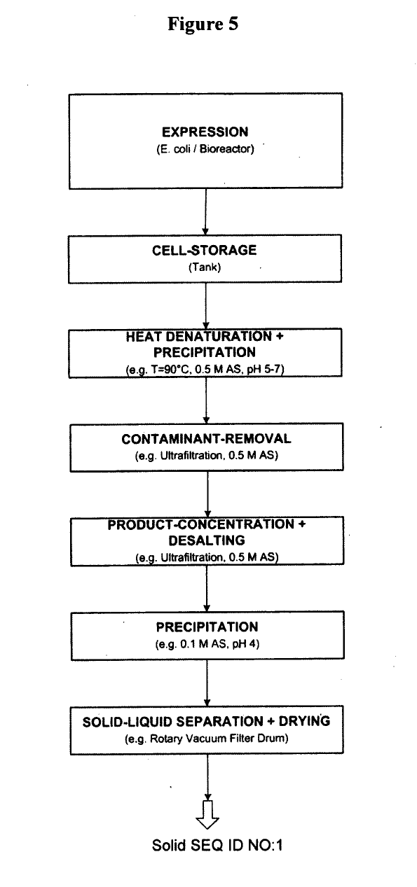

After precipitation, high purity SEQ ID NO:1 protein was isolated by filtration, optionally followed by drying. The protein could be resuspended as necessary. Flow diagrams showing a summary of process steps for polypeptide isolation with heat treatment (FIG. 5) or without heat treatment (FIG. 4) are shown in FIGS. 4 and 5. It is recognized that although independent blocks are shown, these may be combined into a single unit operation where appropriate. It is also recognized that depending on the flowsheet structure that may be chosen during the design phase, some steps such as product concentration and desalting may not be required, particularly in those cases where rotary drum vacuum filtration is used.

### Examples 9-12

### Switching of SEQ ID NO:1-Stabilized Foams Via Three Distinct Mechanisms

For following Examples 9-12, 15 cm high glass columns were used for foam formation. The columns have a glass frit in the base, through which air is bubbled at 1 mL/min via syringe pumps. Air was bubbled for 10 minutes to allow the foams of interest to form, then additions were made as required with pumps continuously running. Unless otherwise specified, all samples had 0.3 mg/mL SEQ ID NO:1 protein, 25 mM HEPES, 10 mM NaCl, with pH and metal ion/metal ion chelator added as required for the test.

### Example 9

### Metal-Ion Switch

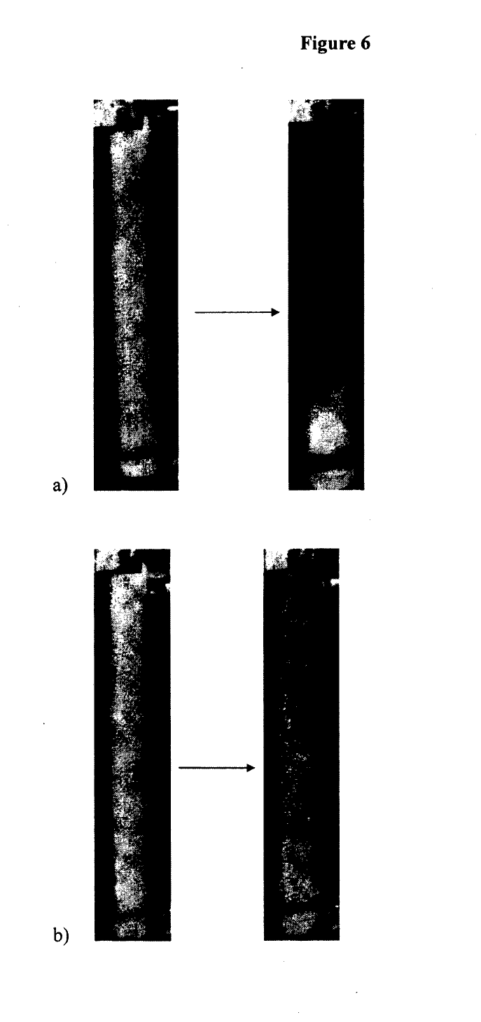

Two identical foams where made by bubbling 1 mL solutions of 0.3 mg/mL SEQ ID NO:1, 10 mM NaCl, 25 mM HEPES pH 7.4, 500 μM Zn⁺⁺ for 10 min at 1 mL/min air flowrate (FIG. 6a and b right image). To one of these foams, 20 μL of 100 mM EDTA was added to chelate zinc ions. After 10 min of further bubbling, the foam with added EDTA had collapsed to a fraction of its height (FIG. 6a), while the control foam (FIG. 6B) had coarsened slightly but remained the same height.

### Example 10

### pH-Switch with Acid

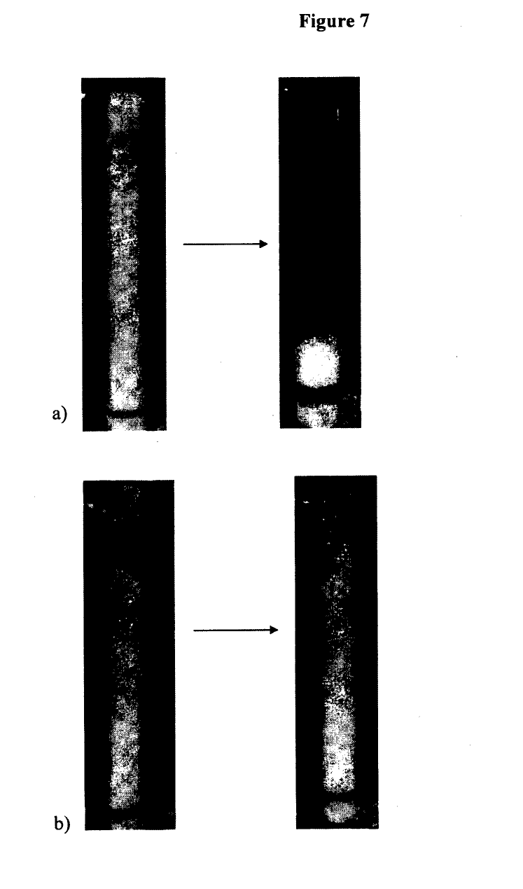

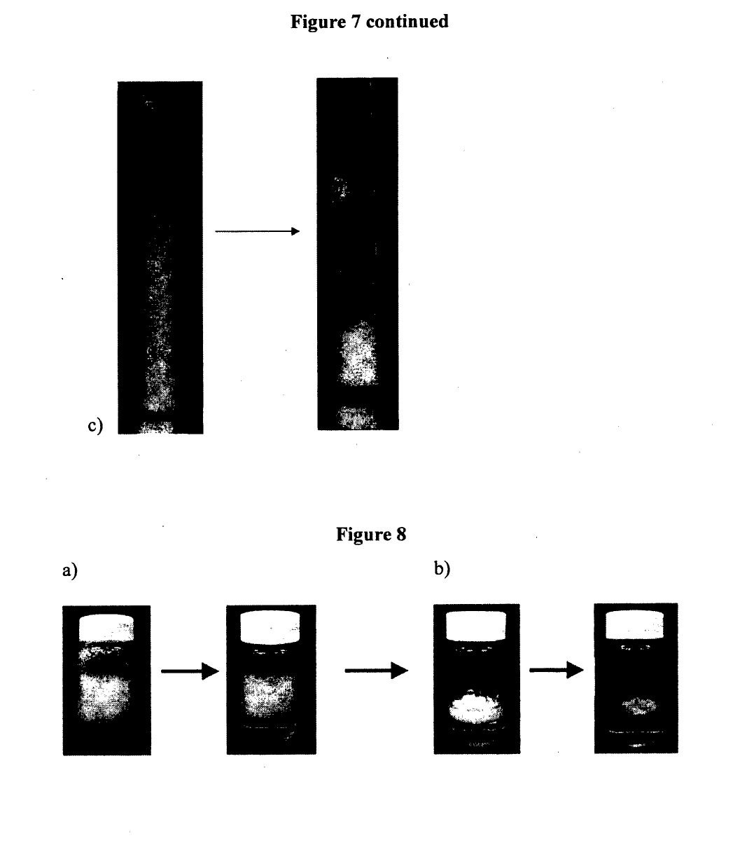

Three identical foams were formed using the same method as in Example 9, however with the initial sample at pH 8.5 and with 200 μM EDTA added to the initial solution. To one of these foams, 14 μL 1 M HCl was added (bulk pH 7.5 after foam collapse). To the second, 14 μL 1 M NaCl was added. To the third, 7 μL 1 M H₂SO₄ was added (bulk pH 7.5 after foam collapse). After 10 min further bubbling of all three foams, the two with changed pH to 7.5 had collapsed to a fraction of their initial height (FIGS. 7a and 7c), while the foam with added NaCl remained unaffected (FIG. 7b).

### Example 11

### pH Switch by Dilution

A sample of 0.3 mg/mL SEQ ID NO:1 in Milli-Q was prepared with 200 μM EDTA, then the pH adjusted to 8.5 with NaOH. The sample was shaken for 30 sec, and formed a fine, stable foam that changed little over 15 minutes (FIG. 8a). The drained liquid was then carefully removed and replaced with the same volume of Milli-Q water, and the sample shaken again for 30 sec. Very little foam remained, and this was not stable (FIG. 8b). The bulk pH had dropped to pH 7.1 due to the dilution step.

### Example 12

### Effect of Kosmotropic and Chaotropic Salts

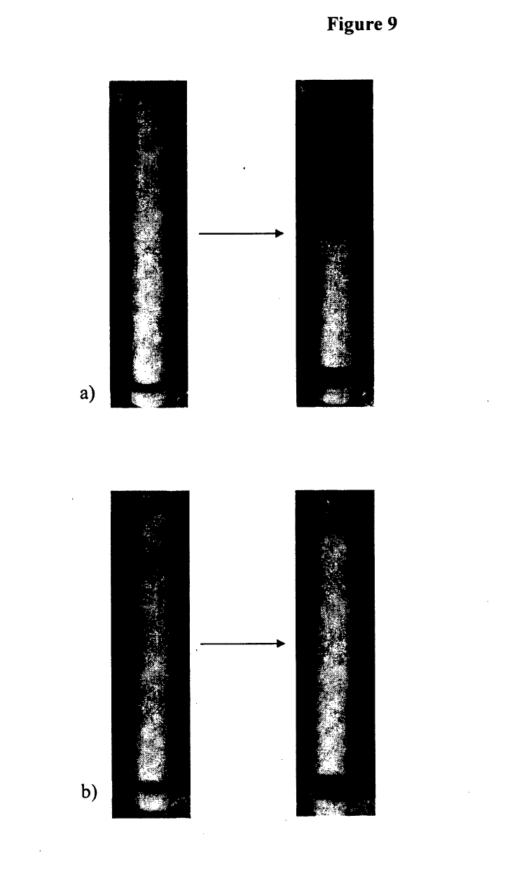

Two identical foams were formed at the same conditions as Example 10 (pH 8.5, 200 μM EDTA) and using the same method. To one of these foams 3 M NaSCN was added to give a bulk concentration of 500 mM, to the other 1 M Na₂SO₄. After 10 mM of continued bubbling, the foam with added NaSCN had collapsed significantly (FIG. 9a) while the foam with added Na₂SO₄ was almost unchanged (FIG. 9b).

### Example 13

### Helical Bulk-Structure of SEQ ID NO:1

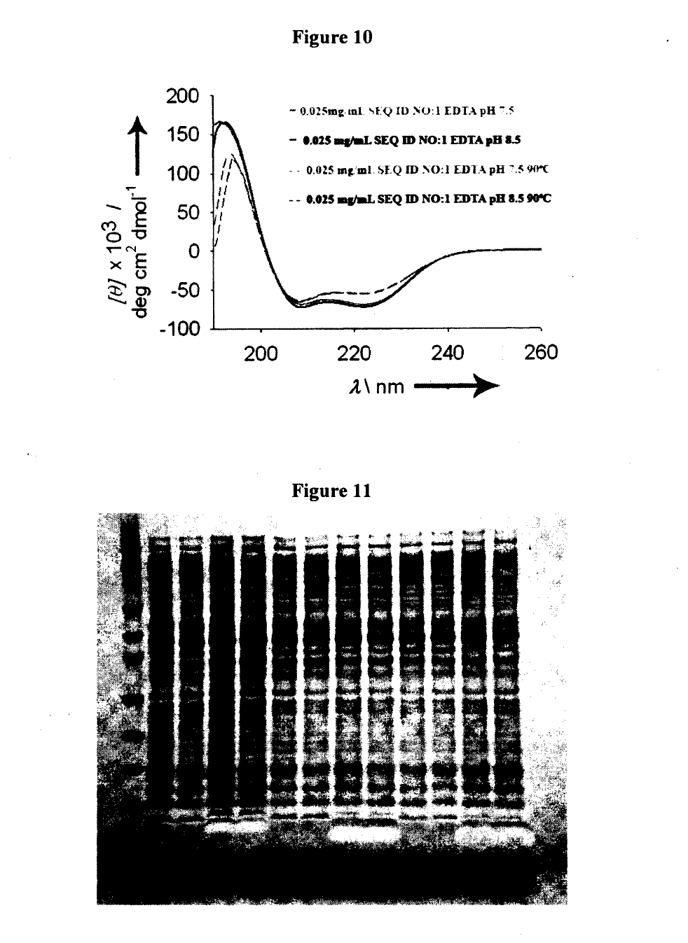

Samples of SEQ ID NO:1 (0.025 mg/mL) were prepared in Milli-Q water with 200 μM EDTA at pH 7.5 and 8.5. A lower concentration of SEQ ID NO:1 was used compared to foaming tests due to the high circular dichroism (CD) signal intensity of SEQ ID NO: 1. HEPES buffer interferes with CD spectra so samples were prepared in Milli-Q water. Each sample was run at 25° C. and then at 90° C. to observe thermal stability. Very high α-helical structure of SEQ ID NO:1 could be observed with no dependence on pH (FIG. 10 solid lines). Slight thermal unfolding was observed, but a large amount of α-helical structure still remained at 90° C., with again no dependence on pH (FIG. 10 dotted lines).

### Example 14

### Design and Expression of Sequences Rich in Glu or Asp (SEQ ID Nos 8, 10 and 11)

Three four-helix bundle sequences were designed as shown in the alignment:

| | | | |
|---|---|---|---|
| SEQ ID 8 | 1 | MDPSAQSVAQSLAQLAQSVSQLVSQADPSAQSVAQSLAQLAQSVSQLVSQADPSAQSVAQ | 60 |
| SEQ ID 10 | 1 | MDPSAQSVAESLAQLAESVSELVSQADPSAQSVAESLAQLAESVSELVSQADPSAQSVAE | 60 |
| SEQ ID 11 | 1 | MDPSANSVAESLANLAESVSELVSNADPSANSVAESLANLAESVSELVSNADPSANSVAE | 60 |
| | | *****.***.***.**.***.***.*****.***.***.**.***.***.*****.***. | |
| SEQ ID 8 | 61 | SLAQLAQSVSQLVSQADPSAQSVAQSLAQLAQSVSQLVSQAD | 102 |
| SEQ ID 10 | 61 | SLAQLAESVSELVSQADPSAQSVAESLAQLAESVSELVSQAD | 102 |
| SEQ ID 11 | 61 | SLANLAESVSELVSNADPSANSVAESLANLAESVSELVSNAD | 102 |
| | | ***.**.***.***.*****.***.***.**.***.***.** | |

For all constructs, a starter culture was grown from a single colony picked from a plate (LB agar-Kan 15 μg/mL). This starter culture was used to inoculate 100 mL of Luria broth containing Kanamycin at 15 μg/mL in a shake flask culture. Cultures were incubated at 37° C. until the OD₆₀₀ reached 0.5, at this point each culture was induced with 1 mM IPTG and further incubated at 26° C. overnight until harvest (16-18 hrs). 1 mL samples were taken at 0 hr and O/N (16-18 hrs) time points. The samples were lysed in Bugbuster (Novagen) to analyze for expression in the total and soluble fractions. The samples were analyzed by SDS-PAGE, as shown in FIG. 11.

### Example 15

### SEQ ID NO:1 Purification by Single Step Thermal Treatment, with and without Ammonium Sulphate

Frozen Escherichia coli cells containing expressed SEQ ID NO:1 prepared in Example 1 were thawed and re-suspended, one sample in Milli-Q water and one sample in HEPES buffer (25 mM HEPES, 20 mM NaCl, pH 7.6), to give a final cell OD₆₀₀ of 20.

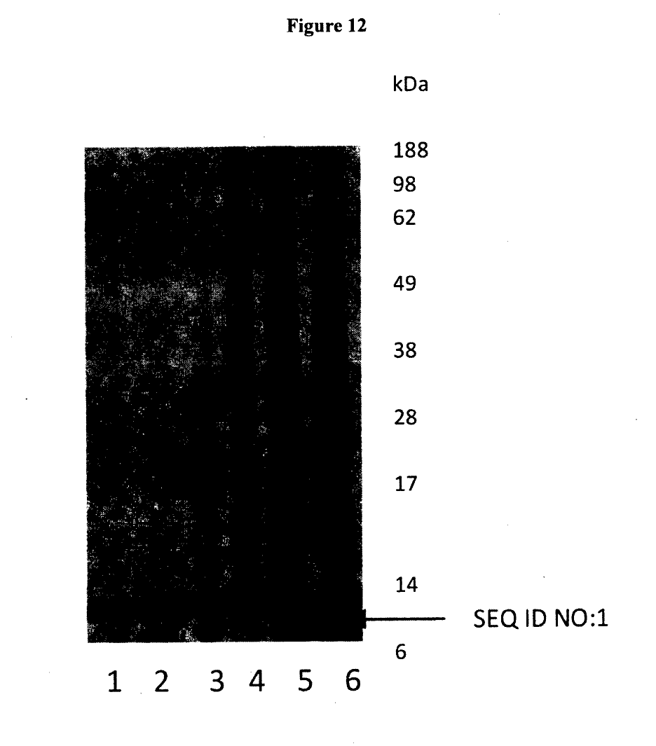

Four samples each of 500 tit cell suspension were used to test the effect thermal treatment (90° C., 20 min) in the presence and absence of AS. In each case, treated material was centrifuged (15000×g, 5 min) after thermal treatment to separate the soluble and insoluble fractions. Supernatant was collected and analyzed by SDS-PAGE (FIG. 12).

FIG. 12 illustrates results of performed thermal treatment experiments (Lanes 1-4), including the soluble protein fraction (Lane 5) without any treatment, following sonication and centrifugation (15000×g, 5 min).

### Sample 1 (Lane 1)

AS was added to the cell suspension prepared with HEPES buffer, to give a final concentration of 1.5 M AS and a final volume of 1 mL. The suspension was heated at 90° C., 20 min. The reaction mixture was centrifuged (15000×g, 5 min); supernatant was collected and analyzed.

### Sample 2 (Lane 2)

AS was added to the cell suspension prepared with Milli-Q water, to give a final concentration of 1.5 M AS with a final volume of 1 mL. The suspension was heated at 90° C., 20 min. The reaction mixture was centrifuged (15000×g, 5 min); supernatant was collected and analyzed.

### Sample 3 (Lane 3)

Cell suspension prepared with HEPES buffer was diluted with HEPES buffer to a final volume of 1 mL. The suspension was heated at 90° C., 20 min. The reaction mixture was centrifuged (15000×g, 5 min); supernatant was collected and analyzed.

### Sample 4 (Lane 4)

Cell suspension prepared with Milli-Q water was diluted with Milli-Q water to a final volume of 1 mL. The suspension was heated at 90° C., 20 min. The reaction mixture was centrifuged (15000×g, 5 min); supernatant was collected and analyzed

The results show the clear importance of including AS in the buffer during heat treatment to improve the purity and recovery of SEQ ID NO: l product in the supernatant.

### Example 16

### SEQ ID NO:11 Purification Using Single-Step Thermal Treatment of Intact Cells

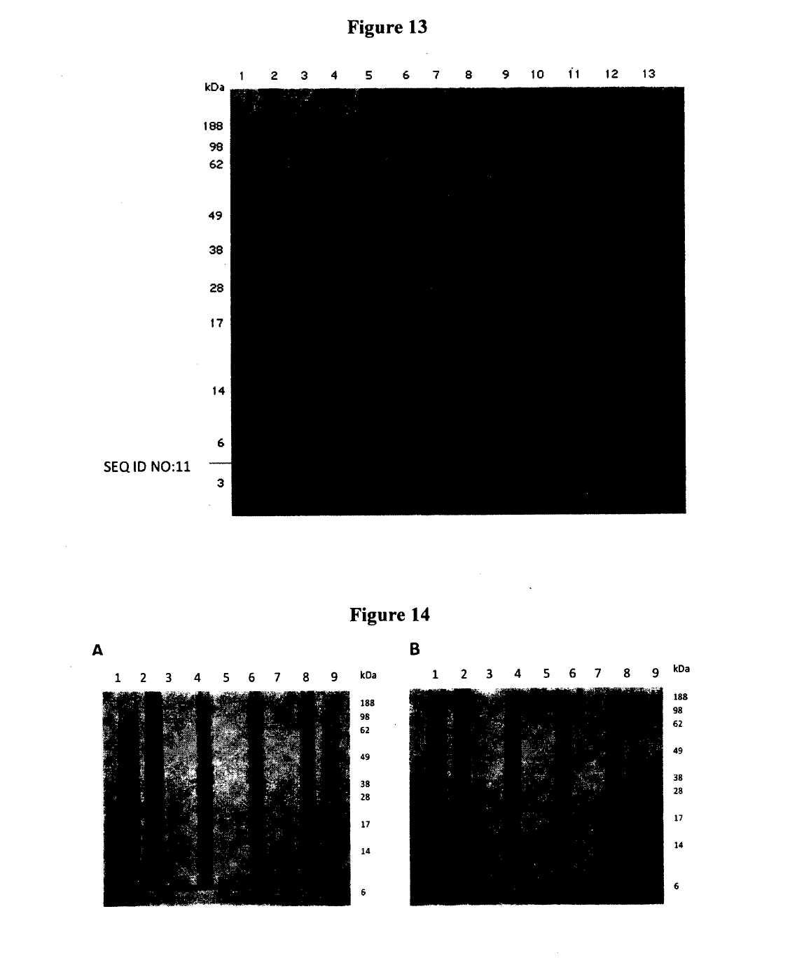

SEQ ID NO:11 (10.1 kDa) was expressed in Escherichia coli by shake flask cultures, using complex medium (LB agar-Kan). Expression involved cultivation at 37° C. and induction for 16 h with IPTG at 26° C. Cells were harvested by centrifugation and re-suspended in Milli-Q water to an OD₆₀₀ₙₘ of 13. FIG. 13 provides an SDS-PAGE analysis of the soluble and insoluble fractions of thermally treated cells re-suspension. Soluble SEQ ID NO:11 can be observed.

Four samples each of 200 μL cell suspension were used to test the effect thermal treatment in the presence of ammonium sulphate (AS) (sample 1=0 M AS, sample 2=0.5 M AS, sample 3=1 M AS, sample 4=1.5 M AS). In each case, treated material was centrifuged (21885×g, 5 min) to separate the soluble and insoluble fractions. Supernatant was collected and the pellet was resuspended in the same volume as the initial sample prior to treatment.

### Sample 1 (Lanes 2, 3, 4 in FIG. 13)

Cell pellet with expressed SEQ ID NO:11 (1000 μL) was resuspended with Milli-Q water to adjust the final volume to 200 μL. The suspension was heated at 90° C. for 20 minutes. The reaction mixture was centrifuged (21885×g, 5 min); supernatant was collected and precipitate was resuspended in 200 μL PBS.

### Sample 2 (Lanes 5, 6, 7 in FIG. 13)

AS was added to the provided 200 μL cell suspension to give a final concentration of 0.5 M. The suspension was heated at 90° C. for 20 minutes. The reaction mixture was centrifuged (21885×g, 5 min); supernatant was collected and precipitate was resuspended in 2004 PBS.

### Sample 3 (Lanes 8, 9, 10 in FIG. 13)

AS was added to 200 μL cell suspension to give a final concentration of 1.0 M. The suspension was heated at 90° C. for 20 minutes. The reaction mixture was centrifuged (21885×g, 5 min); supernatant was collected and precipitate was resuspended in 200 μL PBS.

### Sample 4 (Lanes 11, 12, 13 in FIG. 13)

AS was added to 200 μL cell suspension to give a final concentration of 1.5 M. The suspension was heated at 90° C. for 20 minutes. The reaction mixture was centrifuged (21885×g, 5 min); supernatant was collected and precipitate was resuspended in 200 μL PBS.

### Example 17

### SEQ ID NO:7 Purification by Single Step Thermal Treatment with 1.0 M AS at Varying Heating Times and Temperatures

E. coli cells in fresh culture medium, containing expressed SEQ ID NO:7, were used to perform purification experiments. The cell suspension was concentrated to an OD₆₀₀ₙₘ of 20. Different samples each of 500 μL cell suspension were prepared to test the effect thermal treatment at different heating temperatures and incubation times in the presence of 1.0 M (NH₄)₂SO₄ [AS]. FIG. 14 (A, B) provides an SDS-PAGE analysis of the soluble (lane 1) and insoluble (Lane 2) fractions of a control cell sample following re-suspension and sonication. The following samples of intact cells were prepared and tested using thermal treatment. In all cases the final sample volume was 1.0 mL following addition of AS. After thermal treatment under the given conditions, the reaction mixture was centrifuged (15000×g, 5 min); supernatant was collected and precipitate was re-suspended in 1 mL PBS for SDS-PAGE analysis.

Sample 1: 90° C., 10 min incubation (lanes 3, 4 in FIG. 14A)
Sample 2: 90° C., 30 min incubation (lanes 5, 6 in FIG. 14A)
Sample 3: 90° C., 60 min incubation (lanes 7, 8 in FIG. 14A)
Sample 4: 80° C., 20 min incubation (lanes 3, 4 in FIG. 14B)
Sample 5: 90° C., 20 min incubation (lanes 5, 6 in FIG. 14B)
Sample 6: 100° C., 20 min incubation (lanes 7, 8 in FIG. 14B)

No obvious differences between different heating temperatures and incubation times regarding purity and product yield could be observed. Besides the standard heating conditions of 90° C., 20 minutes, also 80° C., 20 minutes and 90° C., 10 minutes are appropriate to give the same purity and product yield. Generally it can be observed that in the presence of 1.0 M AS the SEQ ID NO:7 solubility is reduced compared to lower AS concentration. Nevertheless, with concentrations of 1.0 M AS better product purities can be achieved compared to lower AS concentrations.

### Example 18

### SEQ ID NO: 1 Purification by Single Step Thermal Treatment, in the Presence of Ammonium Sulphate (AS) or Sodium Sulphate (SS)

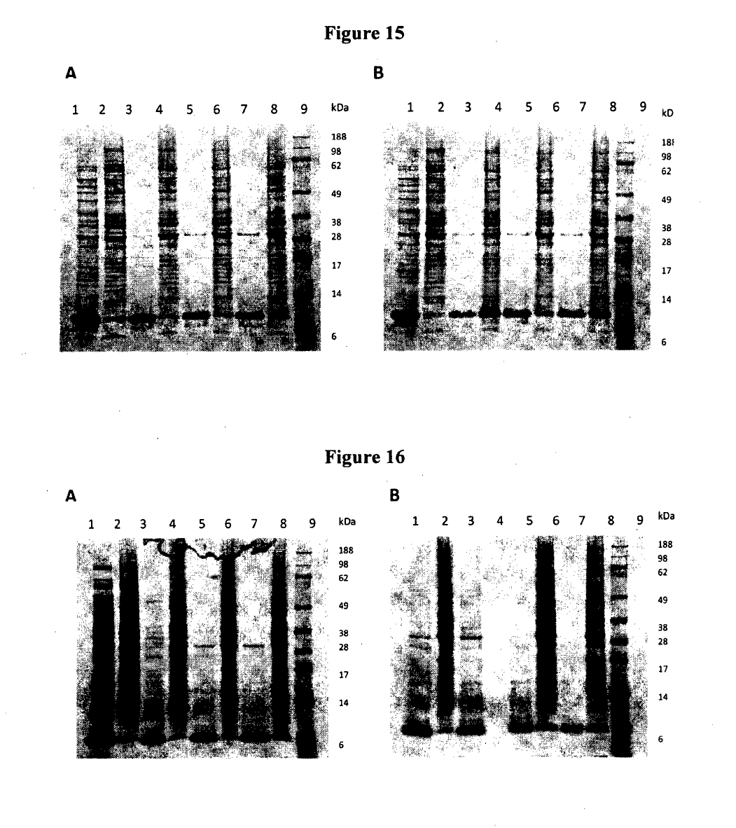

E. coli cells in fresh culture medium, containing expressed SEQ ID NO: 1, were used for purification experiments. The cell suspension was concentrated to an OD₆₀₀ₙₘ of 20. Different samples each of 500 μL cell suspension were prepared to test the effect of thermal treatment at different ammonium sulphate (AS) concentrations (0.25 M, 0.5 M, 1.5 M) and sodium sulfate (SS) concentrations (0.5 M, 1.0 M) on fresh cultivated cells. FIG. 15 (A+B) provides an SDS-PAGE analysis of the soluble (lane 1) and insoluble (lane 2) fractions of sonicated cells following re-suspension and sonication. Also results of soluble and insoluble fractions after thermal treatment with different AS and SS concentrations are illustrated in FIG. 15.

Following Samples were Prepared:

### Sample 1: 0 M AS/SS (Lanes 3, 4 in FIG. 15A)

Cell suspension with expressed SEQ ID NO:1 (500 μL) was diluted with Milli-Q water to adjust the final volume to 1 mL. The suspension was heated at 90° C. for 20 minutes. The reaction mixture was centrifuged (15000×g, 5 min); supernatant was collected and precipitate was re-suspended in 1 mL PBS.

### Sample 2: 0.25 M AS (Lanes 5, 6 in FIG. 15A)

AS was added to the provided cell suspension to give a final concentration of 0.25 M with a final volume of 1 mL. The suspension was heated at 90° C. for 20 minutes. The reaction mixture was centrifuged (15000×g, 5 min); supernatant was collected and precipitate was re-suspended in 1 mL PBS.

### Sample 3: 0.5 M AS (Lanes 7, 8 in FIG. 15A)

AS was added to the provided cell suspension to give a final concentration of 0.5 M with a final volume of 1 mL. The suspension was heated at 90° C. for 20 minutes. The reaction mixture was centrifuged (15000×g, 5 min); supernatant was collected and precipitate was re-suspended in 1 mL PBS.

### Sample 4: 1.5 M AS (Lanes 3, 4 in FIG. 15B)

AS was added to the provided cell suspension to give a final concentration of 1.5 M with a final volume of 1 mL. The suspension was heated at 90° C. for 20 minutes. The reaction mixture was centrifuged (15000×g, 5 min); supernatant was collected and precipitate was re-suspended in 1 mL PBS.

### Sample 5:0.5 M SS (Lanes 5, 6 in FIG. 15B)

SS was added to the provided cell suspension to give a final concentration of 0.5 M with a final volume of 1 mL. The suspension was heated at 90° C. for 20 minutes. The reaction mixture was centrifuged (15000×g, 5 min); supernatant was collected and precipitate was re-suspended in 1 mL PBS.

### Sample 6: 1.0 M SS (Lanes 7, 8 in FIG. 15B)

SS was added to the provided cell suspension to give a final concentration of 1.0 M with a final volume of 1 mL. The suspension was heated at 90° C. for 20 minutes. The reaction mixture was centrifuged (15000×g, 5 min); supernatant was collected and precipitate was re-suspended in 1 mL PBS.

Generally it could be observed that AS or SS was required during thermal treatment to enhance the purity of the SEQ ID NO: 1. The presence of 0.25 M and 0.5 M AS both showed similar good product purity and SEQ ID NO:1 yield, while 1.5 M AS resulted in a higher purity, but lower SEQ ID NO:1 solubility, hence a lower product yield. It can be assumed that the culture medium salts caused an additional salting-out effect on cell proteins and SEQ ID NO:1

Samples of 0.5 M and 1.0 M SS also showed a good product purity and yield. No obvious differences between SS and AS samples regarding purity and product loss could be observed, hence the use of SS is a possible alternative to AS for the SEQ ID NO: 1 purification by thermal treatment.

Concentrations of 0.25 M and 0.5 M AS or SS are determined as appropriate for the effective purification of SEQ ID NO:1 by single step thermal treatment of cells in media.

### Example 19

### SEQ ID NO:7 Purification by Single Step Thermal Treatment, in the Presence of Different Salts ((NH₄)₂SO₄, Na₂SO₄, CaCl₂)

E. coli cells in fresh culture medium, containing expressed SEQ ID NO:7, were used for purification experiments. The cell suspension was concentrated to an OD₆₀₀ₙₘ of 20. Different samples each of 500 μL cell suspension were prepared to test the effect thermal treatment in the presence of low concentration of different salts on fresh cultivated cells, including (NH₄)₂SO₄ (AS), Na₂SO₄ (SS), CaCl₂ (CC). FIG. 16 (A) provides an SDS-PAGE analysis of the soluble (lane 1) and insoluble (lane 2) fractions of sonicated cells following re-suspension and sonication. Also results of soluble and insoluble fractions after thermal treatment with different salt concentrations are illustrated in FIG. 16 (A, B).

Following samples were prepared. In each case, the suspension was heated at 90° C. for 20 minutes. The reaction mixture was then centrifuged (15000×g, 5 min) and supernatant was collected and precipitate was re-suspended in 1 mL PBS prior to SDS-PAGE analysis.

### Sample 1: 0 M (Lanes 3, 4 in FIG. 16A)

Cell suspension was diluted with Milli-Q water to adjust the final volume to 1 mL.

### Sample 2: 0.25 M AS (Lanes 5, 6 in FIG. 16A)

AS was added to the provided cell suspension to give a final concentration of 0.25 M with a final volume of 1 mL.

### Sample 3: 0.5 M AS (Lanes 7, 8 in FIG. 16A)

AS was added to the provided cell suspension to give a final concentration of 0.5 M with a final volume of 1 mL.

### Sample 4: 0.25 M SS (Lanes 1, 2 in FIG. 16B)

SS was added to the provided cell suspension to give a final concentration of 0.25 M with a final volume of 1 mL.

### Sample 5: 0.5 M SS (Lane 3 in FIG. 16B)

SS was added to the provided cell suspension to give a final concentration of 0.5 M with a final volume of 1 mL.

### Sample 6: 0.25 M CC (Lanes 5, 6 in FIG. 16B)

CC was added to the provided cell suspension to give a final concentration of 0.25 M with a final volume of 1 mL.

### Sample 7: 0.5 M CC (Lanes 7, 8 in FIG. 16B)

CC was added to the provided cell suspension to give a final concentration of 0.5 M with a final volume of 1 mL.

Generally it was observed that the addition of different salts led to an increase in the purity of the SEQ ID NO:7 solution, hence an additional salting-out of contaminant proteins could be achieved. For SEQ ID NO:7, SS showed less effectiveness in salting-out impurities than (NH₄)₂SO₄ (AS), hence the purity was lower. No obvious differences between 0.25 M and 0.5 M AS regarding purity could be observed. The presence of CaCl₂ caused a stronger salting-out of proteins, including SEQ ID NO:7. The SEQ ID NO:7 loss seemed to be higher. Nevertheless, 0.25 M and 0.5 M AS or CaCl₂ are appropriate in purifying SEQ ID NO:7 by a single-step thermal treatment showing good recovery yield and purity.

### Example 20

### Preparation of α-Helical Peptides from Polypeptide Biosurfactant

A synthetic gene encoding SEQ ID NO:1 was optimised for E. coli expression (GENEART AG, Germany) and cloned into the pET-48b(+) vector (Novogen®, USA). Chemically competent E. coli BL21(DE3) cells were transformed with this vector and used to express the recombinant SEQ ID NO:1 protein (Protein Expression Facility, University of Queensland, Australia). Briefly, LB plates (Amresco LB agar, Miller formulation, tissue culture grade, Solon, Ohio) containing 15 μg mL⁻¹ kanamycin sulphate (Gibco, Invitrogen, SKU#11815) were streaked and a single colony selected for expression. 50 mL LB medium (Amresco LB broth, Miller formulation, tissue culture grade, Solon, Ohio) containing 15 μg mL⁻¹ kanamycin sulphate was inoculated from the single colony and incubated for 16 hours at 37° C. on an orbital shaker. When OD₆₀₀ reached 0.5, cultures were induced with 1 mM IPTG (isopropyl-β-D-thiogalactopyranoside), then grown further for 4 hours at 37° C. Cell pellets were obtained by centrifugation for 15 minutes at 9000×g and 4° C. (Beckman Coulter—Avanti J-20 XPI) and were then stored at −80° C. until further use. To prepare material for the testing of foams, shake-flask culture methods were translated to a small bioreactor for expression using an established fermentation method (Middelberg et al., Biotechnol. Bioeng., 38, 363-370, 1991). Purification of SEQ ID NO:1 involved standard sequential steps of cell disruption, immobilized metal affinity chromatography (IMAC) relying on the high intrinsic histidine content in SEQ ID NO:1, ion-exchange chromatography (IEX) and reversed-phase chromatographic polishing (RP-HPLC) using a standard water-acetonitrile buffer system (Kaar et al., Biotechnol. Bioeng., 102, 176-187, 2009). Final SEQ ID NO:1-containing fractions recovered from RP-HPLC were lyophylised.

Synthetic SEQ ID NO:12 was synthesized by Genscript Corporation (Piscataway, N.J., USA).

Lyophilized SEQ ID NO:1 was resuspended in 60 mM HCl, then kept sealed in a heating block set at 60° C. for 48 hours. Samples were taken during this 48 hour period to monitor the kinetics of cleavage. After 48 hours, the sample was cooled, neutralized using the required amount of NaOH, then stored at −80° C. until required.

The kinetics of SEQ ID NO:1 cleavage were monitored by analytical reversed-phase chromatography, using an LC-10A VP HPLC system (Shimadzu, Kyoto, Japan), and Jupiter C18 5 mm 300 Å column (Phenomenex, Torrance, Calif.). A linear gradient from 30% to 65% elution buffer in 35 minutes was used at a flow rate of 1 mL min⁻¹. The equilibration buffer was 0.1% trifluroacetic acid (TFA) in milli-Q water, and the elution buffer was 90% acetonitrile, 0.1% TFA.

To identify the components present in the sample after cleavage of SEQ ID NO:1, electrospray mass spectrometry was used, specifically a Waters Quattro Micro API quadrupole mass spectrometer (Waters, UK) with electrospray ionization (ESI) in positive ion mode. To prepare for mass-spectrometry, a sample of cleaved product was thawed, then run through HPLC (as above) to desalt, and all peaks collected in one pool. To remove solvent, the collected sample was frozen at −80° C. overnight, then lyophilized. The lyophilized cleaved product was resuspended in a minimal amount of Milli-Q to ensure maximum signal, then directly injected into the mass spectrometer at a flowrate of 10 μL min⁻¹. Control and data collection was performed by the Waters MassLynx software, and manually analyzed.

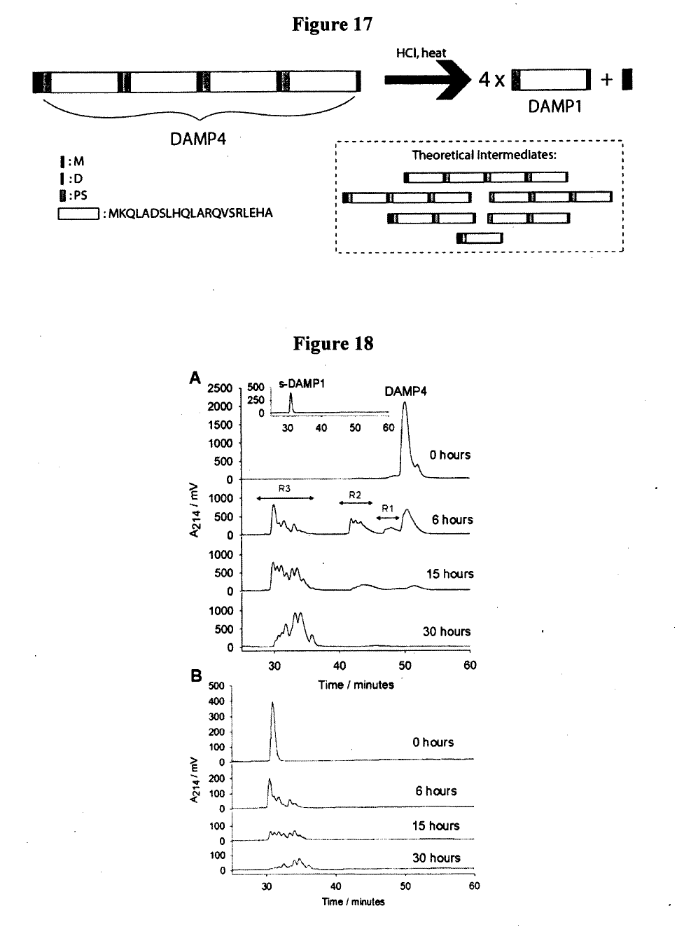

There are four acid cleavage (DP, aspartyl-proline) sites designed into SEQ ID NO:1, one at the start of the sequence, and one between each of the four repeating α-helical peptides. For consistency of cleavage product, an aspartate (D) has been added to the end of the sequence. For expression purposes, the sequence starts with a methionine (M). Theoretically, SEQ ID NO:1 should only be cleaved at the four DP sites, producing a product identical to the synthetically produced SEQ ID NO:12. It would be expected that not all three sites are cleaved simultaneously, therefore reaction intermediates, specifically the trimer and dimer would exist on the pathway to complete cleavage as shown in FIG. 17.

The kinetics of acid cleavage of SEQ ID NO:1 at 60° C. and 60 mM HCl are shown by HPLC in FIG. 18A. Initially, only SEQ ID NO:1 is present (0 hours). After 6 hours, the appearance of three new ‘regions’ of peaks can be seen, R1, around 47-49 minutes, R2, around 42-45 minutes, and R3, comprised of several distinct peaks, at 28-35 minutes. At 15 hours, SEQ ID NO:1 and the first two regions (R1 and R2) have almost disappeared. More peaks have developed in the R3 range. After 30 hours, SEQ ID NO:1 is completely consumed, and only peaks in the R3 range remain, however the range has shifted to about 30-37 minutes. If cleavage had only occurred at the designed sites and with no further effects on amino acids, three distinct, single peaks should have formed. However it is immediately apparent that this is not the case. The inset in FIG. 18 shows the HPLC trace of the synthetically-produced SEQ ID NO:12, for comparison. The SEQ ID NO:12 trace is a single, distinct peak at around 31 minutes. It appears that the acid and heat are having some other effects on the SEQ ID NO:1 sequence, resulting in a heterogeneous mix of components with elution time close to, but varying from, synthetic SEQ ID NO:12.

The effect of the same heat and acid treatment on synthetically produced SEQ ID NO:12 is shown in FIG. 18B. A HPLC trace is produced with very similar features to that of the SEQ ID NO:1 cleavage product. This provides further evidence that the combination of high temperature and acidic conditions are affecting the sequence in some way other than just cleavage at the DP-residues.

To investigate what the other effects on the sequence are, electrospray mass-spectrometry of the final SEQ ID NO:1 cleavage product, was performed. This clear mass spectrum was obtained with a concentrated and desalted cleavage product sample, obtained by freeze drying, lyophilizing, and then resuspending in milli-Q water. Rather than one group of peaks representing a single molecular mass, two groups of peaks, (four peaks per group) are evident—representing that two distinct molecular masses were detected. Analysis of the m/z values showed that the two molecular masses associated to the first and second groups were 2731-2734 Da and 2615.9-2618.9 Da respectively. The theoretical molecular mass of SEQ ID NO:12 is 2731 Da, so the first group represents SEQ ID NO:12 and some other variants of close molecular mass. This indicates that cleavage at the aspartyl-proline (DP) residues is occurring successfully, however with some other effects occurring. The second group molecular mass is 115.1 Da less than that of intact SEQ ID NO:12. This indicates that cleavage of the terminal aspartate (D) from the SEQ ID NO:12 is also occurring, in parallel with the desired DP cleavage.

Upon closer inspection, it was seen that each peak was in fact made up of several peaks with very close m/z values. This very small change in mass is hard to detect in the overall spectrum, but can be seen when zooming in on the individual peaks as demonstrated in the inset for the 545-550 m/z region. Analysis showed that this observed peak broadness can be almost completely accounted for by three minor mass variations, an addition of 1, 2, and 3 Da to both SEQ ID NO:12 and SEQ ID NO:12 without the N-terminal aspartyl group. The same components were present in the synthetically produced SEQ ID NO:12 heat and acid treated sample as in the SEQ ID NO:1 heat and acid treated sample.

A known amino acid modification that causes a mass change of +1 Da is deamidation. Glutamine deamidation is known to occur under acidic conditions, and as there are three glutamines per SEQ ID NO:12, it is likely that this is the source of the 1, 2, and 3 Da mass variance observed.

Deamidation and aspartyl loss affect the HPLC retention time of the peptide. Glutamic acid has a higher retention coefficient (ie. slows down peptide retention time) under similar HPLC conditions compared to glutamine, which is observed here, the peaks generally shift to the right with increased time of reaction. It also appears that the loss of the terminal aspartate delays retention time.

By acid and heat treating SEQ ID NO:1 under the conditions described, the desired cleavage at the designed DP-residues occurs to produce SEQ ID NO:12 as desired, however due to terminal-D cleavage and glutamine deamidation reactions happening in parallel, this cleavage product differs from pure SEQ ID NO:12. However, the cleavage conditions may be optimised to maintain sufficient cleavage and reduce terminal aspartate cleavage and/or glutamine (or asparagine when present) deamidation.

### Example 21

### Interfacial Tension of SEQ ID NO:1, SEQ ID NO:12 and Mixtures Thereof

A DSA-10 drop-shape analysis unit was used (Krüss GmbH, Hamburg, Germany) to measure interfacial tension kinetics. An 8 mL sample of 8 μM SEQ ID NO:1, SEQ ID NO:12, or a mix of the two at 25, 50, 75, and 90% w/w SEQ ID NO:1, in 25 mM HEPES, 200 μM EDTA, pH 7.4. A quartz cuvette (Hellma GmbH, Mülheim, Germany) was used to hold samples. Bubbles were formed through a u-shaped stainless steel capillary of known diameter fed by a glass syringe operated manually. Prior to use, cleanliness and operation of the system was checked by forming an air bubble in milli-Q water, and confirming a constant interfacial tension of 72.8 mN m⁻¹ for 10 minutes. To measure the interfacial tension kinetics of formulated mixtures of SEQ ID NO:12 and SEQ ID NO:1, the cuvette was filled with the sample of interest, a bubble of about 10 μL was formed, and interfacial tension as extracted by the software (via images of the bubble collected by a connected camera), monitored at a rate of about 1 measurement per second.

The air-water interfacial tension (IFT) kinetics to 300 sec of SEQ ID NO:12 at concentrations 2.4, 4.8, 6.4, 7.4, and 8 μM were observed. At 2.4 μM SEQ ID NO:12, a significant lag time of about one minute is observed prior to a rapid decrease in IFT to a final value of 53.8±0.2 mN/m. At all other concentrations, the lag time is not as noticeable and rapid. IFT decrease is complete within less than a minute. The IFT values reached are similar, between 52-53 mN/m. While an effect of concentration on rate of IFT decrease does exist, it is not substantial at the observed time scale.

The interfacial tension kinetics of SEQ ID NO:1 at concentrations of 0.6, 1.6, 3.2, 5.6, and 8 μM up to a time of five minutes were observed. It was immediately noted that the SEQ ID NO:1 adsorption at the interface is much slower than that of SEQ ID NO:12, and the effect of concentration is much more noticeable. As concentration increases, the rate of interfacial tension decrease (adsorption) is also increased. Unlike SEQ ID NO:12, which at all concentrations tested reaches a plateau in interfacial adsorption within 5 minutes, SEQ ID NO:1 does not reach equilibrium within this timeframe at the concentrations tested. Compared with 8 μM SEQ ID NO:12, which takes about 1 minute to reach its final value, 8 μM SEQ ID NO:1 takes over 5 minutes. This concentration is on a per molecule-basis and would be four times greater on a per-monomer basis. Essentially, the amount of mass in 8 μM of SEQ ID NO:1 is four times that in 8 μM SEQ ID NO:12 (Table 3). The theoretical diffusion time constants, t_{d}, for SEQ ID NO:12 and SEQ ID NO:1 were calculated and compared to those experimentally observed (Table 4). As can be seen in Table 4, adsorption of SEQ ID NO:12 appears to be diffusion-controlled as the theoretical t_{d} values are close to those observed experimentally. Adsorption of SEQ ID NO:1 however is much slower than predicted by the diffusion time constants, indicating the significant energy barrier to adsorption exists for SEQ ID NO:1 that is not present for SEQ ID NO:12. SEQ ID NO:1 folds into a very stable 4-helix bundle in bulk, therefore the slow adsorption rates observed are likely to be due to a significant energy barrier associated with the unfolding of this 4-helix bundle in order for adsorption to occur.

TABLE 3
Experimental concentrations of SEQ ID NO: 12 and SEQ ID NO: 1.

| Mass basis | | | | Molar Basis | | | |
|---|---|---|---|---|---|---|---|
| | [SEQ ID | [SEQ ID | | | [SEQ ID | [SEQ ID | |
| % SEQ | NO: 1] μg | NO: 12] | Total | % SEQ | NO: 1] | NO: 12] | Total |
| ID NO: 1 | mL⁻¹ | μg mL⁻¹ | μg mL⁻¹ | ID NO: 1 | μM | μM | μM |
| 100 | 88.9 | — | 88.9 | 100 | 8 | — | 8 |
| 90 | 62.3 | 6.6 | 68.8 | 67 | 5.6 | 2.4 | 8 |
| 75 | 35.6 | 13.1 | 48.7 | 42 | 3.2 | 4.8 | 8 |
| 50 | 17.8 | 17.5 | 35.3 | 19.5 | 1.6 | 6.4 | 8 |
| 25 | 6.7 | 20.2 | 26.9 | 7.5 | 0.6 | 7.4 | 8 |
| 0 | — | 21.8 | 21.8 | 0 | — | 8 | 8 |

TABLE 4
Theoretical and approximate experimental diffusion time constants (t_{D}) for SEQ
ID NO: 1 and SEQ ID NO: 12

| SEQ ID NO: 1 | | | | SEQ ID NO: 12 | | |
|---|---|---|---|---|---|---|
| Concentration, | Concentration, | | Approx. | Concentration, | | Approx. |
| μM | μM monomer | Theoretical | experimental | μM | Theoretical | experimental |
| SEQ ID NO: 1 | equivalent | t_{d} | t_{d} | SEQ ID NO: 12 | t_{d} | t_{d} |
| 0.6 | 2.4 | 488.3 | >1000 | 2.4 | 391.2 | 150 |
| 1.6 | 6.4 | 68.7 | >1000 | 4.8 | 97.8 | 50 |
| 3.2 | 12.8 | 17.2 | >1000 | 6.4 | 55 | 40 |
| 5.6 | 22.4 | 5.6 | >800 | 7.4 | 41.1 | 30 |
| 8 | 32 | 2.8 | >600 | 8 | 35.2 | 25 |

The interfacial tension kinetics at the air-water interface for a SEQ ID NO:12 and SEQ ID NO:1 mixed system were also observed. The mix conditions chosen corresponded to 90%, 75%, 50%, and 25% SEQ ID NO:1 on a mass basis, as shown in Table 3. The total molar concentration was kept constant at 8 μM under all conditions. Interestingly, the interfacial tension kinetics (IFT) under all mix conditions were similar, dropping rapidly within the first 30 seconds to final values of 52-53 mN/m. It appears that SEQ ID NO:1 and SEQ ID NO:12 are cooperating to speed up the overall interfacial tension kinetics compared to the individual systems. The 90% SEQ ID NO:1 condition (5.6 μM SEQ ID NO:1 2.4 μM SEQ ID NO:12) shows this most clearly, as the kinetics of the individual components (5.6 μM SEQ ID NO:1 and 2.4 μM SEQ ID NO:12) are much slower than when combined. This is the case for all mix conditions tested, however is less visible on the time-scale displayed.

Without wishing to be bound by theory, the cause of this cooperative effect is likely due to the fact that SEQ ID NO:1 is a 4× repeat of the SEQ ID NO:12 sequence. Essentially, adding SEQ ID NO:1 to a solution adds the equivalent of four SEQ ID NO:12 peptides. Therefore, the total effective SEQ ID NO:12-peptide bulk concentration in the mixed systems is [SEQ ID NO:12]+4× [SEQ ID NO:1]. Additionally, the total mass in the system increases as the proportion of SEQ ID NO:1 increases (as total moles were kept constant, Table 3), therefore an increase in rates of interfacial tension would be expected.

### Example 22

### Foaming Stability of SEQ ID NO:1, SEQ ID NO:12 and Mixtures Thereof

A previously described in Examples 9-12 foaming apparatus was used to compare foaming of 0.3 mg/mL SEQ ID NO:12, 0.3 mg/mL SEQ ID NO:1, and 0.15 mg/mL SEQ ID NO:12+0.15 mg/mL SEQ ID NO:1 in 25 mM HEPES, 200 uM EDTA, pH 8.5. 0.5 mL of sample was bubbled at 1 mL min⁻¹ using syringe pumps connected to a glass column of 15 cm height and 1 cm diameter, fitted with a sintered glass frit at the base. Air was pumped for 10 minutes, then the pumps turned off and foams observed for 1 hour to compare stability.

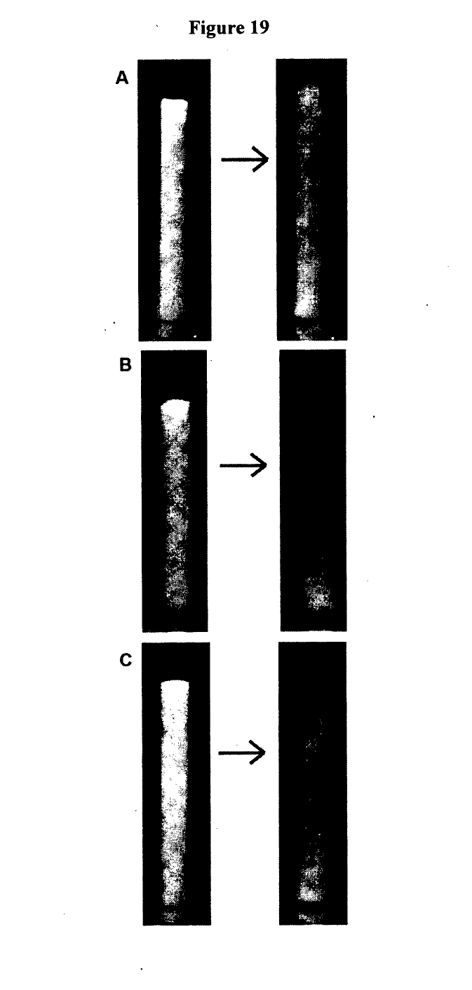

Both SEQ ID NO:12 and SEQ ID NO:1 form very substantial, dense foams as shown in FIG. 19. The SEQ ID NO:12 foam is denser than the SEQ ID NO:1 foam, most likely do the slower diffusion rates of SEQ ID NO:1 to the interface. Over the timespan of an hour, the SEQ ID NO:12 foam is much more stable, only coarsening while the SEQ ID NO:1 foam collapses significantly (FIGS. 19A and 19B respectively).

FIG. 19C shows the foaming results under the same conditions for a 50:50 mix of SEQ ID NO:12 and SEQ ID NO:1. Initially, the foam is as tall and dense as that formed by SEQ ID NO:12 alone. After 1 hour, the foam has deteriorated more than the SEQ ID NO:12 foam, but is much more stable than the SEQ ID NO:1 alone foam. This shows that the kinetic stability of a foam can be tuned by the foam formulation, changing only the molecule length.

### Example 23

### Foaming Stability with Varying SEQ ID NO:1 and SEQ ID NO: 12 Ratio

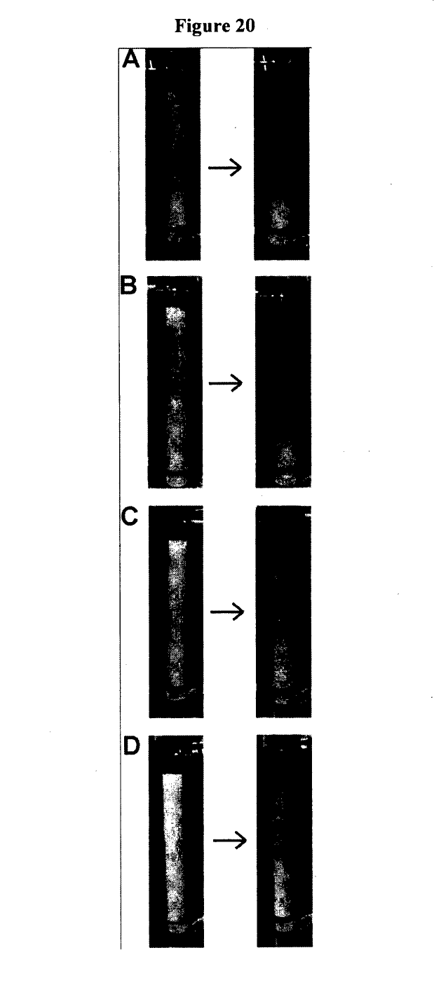

Four foams of varying SEQ ID NO:1: SEQ ID NO:12 ratios were prepared by bubbling air through 1 mL sample in the same foam column as used in Examples 9-12. All samples were in 10 mM NaCl, 200 μM EDTA, 25 mM HEPES, pH 8.5. 0.3 mg/mL SEQ ID NO:1 forms a substantial foam after 10 minutes of bubbling air which coalesced to less than half its original height after 1 hour of standing (FIG. 20A). The initial foam quality increased (ie. bubble size decreased) and foam stability after 1 hour improved when a small amount of SEQ ID NO:12 was added (0.03 mg/mL SEQ ID NO:12, 0.27 mg/mL SEQ ID NO:1—FIG. 20B). Initial bubble size decreased further with increase of added SEQ ID NO:12, and the 1 hour stability improved further (FIG. 20C—0.1 mg/mL SEQ ID NO:12, 0.2 mg/mL SEQ ID NO:1, and FIG. 20D—0.2 mg/mL SEQ ID NO:12, 0.1 mg/mL SEQ ID NO:1).

### Example 24

### Acid Stimulated Switching of a Foam Composition Comprising Both SEQ ID NO:1 and SEQ ID NO:12

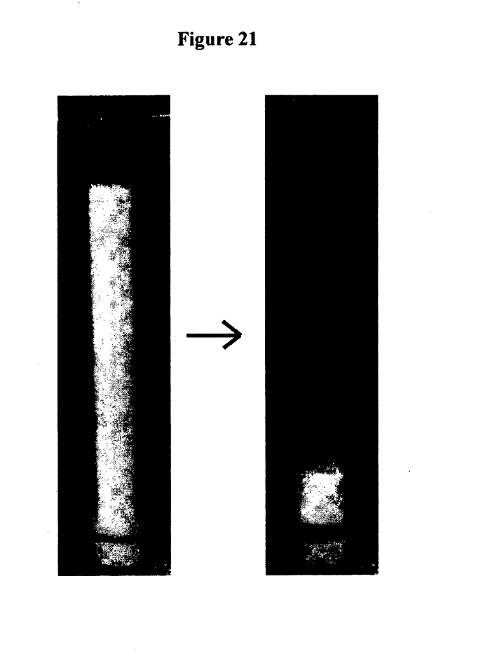

As previously described in Examples 9-12, foaming apparatus was used to prepare foams. A very substantial foam was formed by bubbling air for 10 minutes through 1 mL of 0.15 mg/mL SEQ ID NO:1, 0.15 mg/mL SEQ ID NO:12, 10 mM NaCl, 200 μM EDTA, 25 mM HEPES, pH 8.5 (FIG. 21, left image). 14 uL 1M HCl was added to neutralise the bulk solution to pH 7.4 while keeping maintaining the air flow. The addition of acid dissipated the foam to a fraction of its original height in less than two minutes (FIG. 21, right image).

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1-75. (canceled)

76. A polypeptide or protein comprising at least two α-helical peptides linked by a linking sequence of 3 to 11 amino acid residues, wherein the protein or polypeptide has a folded tertiary structure with a hydrophobic core and a hydrophilic surface; and
wherein each α-helical peptide comprises a sequence of amino acid residues:
(a b c d d′ e f g)ₙ
wherein n is an integer from 2 to 12;
amino acid residues a and d are hydrophobic amino acid residues;
amino acid residue d′ is absent or is a hydrophobic amino acid residue;
at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
amino acid residue g is any amino acid residue;
wherein
i) each α-helical peptide comprises at least one stimuli-responsive amino acid residue; or
ii) each sequence (a b c d d′ e f g) in the α-helical peptide comprises at least one glutamine or asparagine residue and no net charge or each sequence (a b c d d′ e f g) comprises at least one glutamine or asparagine residue and one negative charge.

77. The polypeptide or protein according to claim 76 wherein the at least one stimuli-responsive amino acid residue is a lysine residue.

78. The polypeptide or protein according to claim 76 wherein the at least one stimuli-responsive amino acid residue is a histidine residue.

79. The polypeptide or protein according to claim 76 wherein the at least one stimuli-responsive amino acid residue results from each sequence (a b c d d′ e f g) in the α-helical peptide having a net negative or positive charge.

80. The polypeptide or protein according to claim 76 wherein each sequence (a b c d d′ e f g) in the α-helical peptide comprises at least two glutamine or asparagine residues and no net charge.

81. The polypeptide or protein according to claim 76 wherein each sequence (a b c d d′ e f g) in the α-helical peptide comprises at least one glutamine or asparagine residues and one negative charge.

82. The polypeptide or protein according to claim 76 wherein the linking sequence has 3 to 5 amino acid residues.

83. The polypeptide or protein according to claim 82 wherein the linking sequence has 3 amino acid residues.

84. The polypeptide or protein according to claim 76 wherein the linking sequence comprises a cleavable bond.

85. The polypeptide or protein according to claim 84 wherein the cleavable bond is an acid cleavable bond.

86. The polypeptide or protein according to claim 85 wherein the acid cleavable bond is a D-P bond.

87. The polypeptide or protein according to claim 85 wherein the linking sequence comprises the sequence D-P-S.

88. The polypeptide or protein according to claim 87 wherein the linking sequence is D-P-S.

89. A method of modulating the stability of foam comprising a protein or polypeptide biosurfactant at a liquid-gas interface; wherein said biosurfactant comprises at least two α-helical peptides linked by a linking sequence of 3 to 11 amino acid residues, and wherein each α-helical peptide comprises a sequence of amino acid residues:
(a b c d d′ e f g)ₙ
wherein n is an integer from 2 to 12;
amino acid residues a and d are hydrophobic amino acid residues;
amino acid residue d′ is absent or is a hydrophobic amino acid residue;
at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
amino acid residue g is any amino acid residue;
and wherein each α-helical peptide comprises a stimuli-responsive amino acid residue;
said method comprising the step of:
i) exposing the biosurfactant to a stimulus that alters the zeta potential and/or surface charge of the biosurfactant at the liquid-gas interface or the metal ion binding of the biosurfactant or hydration structure of the biosurfactant at the liquid-gas interface.

90. The method according to claim 89 wherein the α-helical peptide of the protein or polypeptide biosurfactant comprises at least one lysine residue.

91. The method according to claim 89 wherein the stimulus that alters the zeta potential and surface charge of the biosurfactant at the liquid-gas interface alters the pH of the foam.

92. The method according to claim 89 wherein the stimulus is an acid or a base.

93. The method according to claim 89 wherein the pH is altered by dilution of bulk aqueous phase from which the foam is formed.

94. The method according to claim 89 wherein the α-helical peptide of the protein or polypeptide biosurfactant comprises at least one histidine residue.

95. The method according to claim 89 wherein the stimulus alters the metal ion binding of the biosurfactant.

96. The method according to claim 95 wherein the stimulus is a metal ion or a chelating agent.

97. The method according to claim 89 wherein each sequence (a b c d d′ e f g) in the α-helical peptide of the protein or polypeptide biosurfactant has a net negative or positive charge at a specified pH or has no net charge at a specified pH.

98. The method according to claim 97 wherein the stimulus alters the hydration structure of the biosurfactant at the liquid-gas interface.

99. The method according to claim 98 wherein the stimulus is a kosmotropic or chaotropic salt.

100. The method according to claim 89 wherein the stimulus stabilizes or maintains the foam.

101. The method according to claim 89 wherein the stimulus destabilizes the foam or causes it to collapse.

102. The method according to claim 89 further comprising the step of:
ii) exposing the biosurfactant to a second stimulus that alters the zeta potential and/or surface charge of the biosurfactant at the liquid-gas interface or the metal ion binding of the biosurfactant or hydration structure of the biosurfactant at the liquid-gas interface adopted on exposure to the stimulus in step i).

103. The method according to claim 102 wherein steps i) and/or ii) are repeated one or more times.

104. The method according to claim 89 wherein the foam further comprises an a-helical peptide comprising the amino acid sequence:
X₁-(a b c d d′ e f g)ₙ-X₂
wherein n is an integer from 2 to 12;
amino acid residues a and d are hydrophobic amino acid residues;
amino acid residue d′ is absent or is a hydrophobic amino acid residue;
at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
amino acid residue g is any amino acid residue;
X₁ and X₂ are each independently absent or are amino acid residues from a cleavable linking sequence of the polypeptide or protein of claim 1 and wherein the number of amino acid residues in X₁ and X₂ is 3 to 11 amino acid residues and wherein at least one amino acid residue is a stimuli-responsive amino acid residue.

105. The method according to claim 104 wherein the at least one stimuli-responsive amino acid residue is a lysine residue.

106. The method according to claim 104 wherein the at least one stimuli-responsive amino acid residue is a histidine residue.

107. The method according to claim 104 wherein the at least one stimuli-responsive amino acid residue results from each sequence (a b c d d′ e f g) in the a-helical peptide having a net negative or positive charge.

108. The method according to claim 104 wherein each sequence (a b c d d′ e f g) in the α-helical peptide comprises at least two glutamine or asparagine residues and no net charge.

109. The method according to claim 104 wherein each sequence (a b c d d′ e f g) in the α-helical peptide comprises at least one glutamine or asparagine residues and one negative charge.

110. The method according to claim 104 wherein X₁ comprises a proline residue and X₂ comprises an aspartyl residue.

111. The method according to claim 110 wherein X₁ comprises the sequence P-S and X₂ comprises an aspartyl residue.

112. The method according to claim 111 wherein X₁ is P-S and X₂ is D.

113. The method according to claim 89 wherein the foam further comprises an antimicrobial peptide.

114. The method of modulating the stability of a foam comprising the steps of:
i) forming a stable foam from a foaming composition; said foaming composition comprising:
a. a first bulk aqueous phase having a pH of 8.3 or above;
b. a biosurfactant having at least two α-helical peptides linked by a linking sequence of 3 to 11 amino acid residues, wherein each α-helical peptide comprises a sequence of amino acid residues:
(a b c d d′ e f g)ₙ
wherein n is an integer from 2 to 12;
amino acid residues a and d are hydrophobic amino acid residues;
amino acid residue d′ is absent or is a hydrophobic amino acid residue;
at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
amino acid residue g is any amino acid residue;
wherein each α-helical peptide comprises a lysine residue;
ii) removing a substantial fraction of the first bulk aqueous phase from the foaming composition; and
iii) replacing the first bulk aqueous phase with a second bulk aqueous phase having a pH below 8.

115. The method according to claim 114 wherein the first bulk aqueous phase has a pH of about 8.3 to 9.0.

116. The method according to claim 114 wherein the second bulk aqueous phase has a pH of about 7.0 to 7.7.

117. The method according to claim 114 wherein the foam composition further comprises an α-helical peptide, wherein said α-helical peptide comprises a lysine residue.

118. The method according to claim 114 wherein the foam composition further comprises an antimicrobial peptide or a protease, amylase, lipase or cellulase enzyme.

119. A method of purifying a polypeptide or protein biosurfactant that has a folded tertiary structure with a hydrophobic core and a hydrophilic surface; said method comprising the steps of:
i) treating a composition comprising the polypeptide or protein and other cell based protein, polypeptide and/or peptide contaminants with a kosmotropic salt in an amount suitable to salt-out the contaminants to form a precipitate and to salt-in the polypeptide or protein in solution, wherein the treatment is at a temperature of above 45° C.; and
ii) separating the precipitate from the solution containing the polypeptide or protein.

120. The method according to claim 119 wherein step i) is performed at atmospheric pressure and a temperature above 60° C.

121. The method according to claim 120 wherein step i) is performed at a temperature in the range of 85° C. to 100° C.

122. The method according to claim 119 wherein the kosmotropic salt is a sulphate.

123. The method according to claim 122 wherein the kosmotropic salt is ammonium sulphate or sodium sulphate.

124. The method according to claim 122 wherein the amount of kosmotropic salt is in the range of 0.2 M to 1.5 M.

125. The method according to claim 119 wherein the folded tertiary structure is a four helix bundle.

126. A method of manufacturing a polypeptide or protein that has a folded tertiary structure with a substantially hydrophobic core and a substantially hydrophilic surface; said method comprising:
i) providing a microorganism containing a polynucleotide sequence, wherein the polynucleotide sequence comprises a nucleotide sequence that encodes the polypeptide or protein, and wherein the nucleotide sequence is operably linked to a promoter sequence;
ii) culturing the microorganism to express the polypeptide or protein;
iii) disrupting the microorganism cells to form a cell disruptate composition;
iv) treating the disruptate with a kosmotropic salt in an amount suitable to salt-out cell based protein, polypeptide and peptide contaminants to form a precipitate and salt-in the polypeptide or protein in solution, wherein the treatment is at a temperature of above 45° C.; and
v) separating the precipitate from the solution of polypeptide or protein.

127. The method according to claim 126 wherein steps iii) and iv) are performed concurrently at atmospheric pressure and a temperature of at least 60° C.

128. The method according to claim 127 wherein the temperature is in the range of 85° C. to 100° C.

129. The method according to claim 126 wherein the kosmotropic salt is a sulphate.

130. The method according to claim 129 wherein the kosmotropic salt is ammonium sulphate or sodium sulphate.

131. The method according to claim 130 wherein the amount of kosmotropic salt is in the range of 0.2 M to 1.5M.

132. The method according to claim 126 wherein the folded tertiary structure is a four helix bundle.

133. A method of purifying a polypeptide or protein that has a folded α-helical tertiary structure with a substantially hydrophilic surface; the method comprising the step of:
i) treating a composition comprising microorganism cells containing the polypeptide or protein with a kosmotropic salt at a temperature of at least 45° C.

134. A method according to claim 133 wherein step i) is performed at atmospheric pressure and a temperature of at least 60° C.

135. The method according to claim 134 wherein the temperature is in the range of 85° C. to 100° C.

136. A method according to claim 135 wherein step i) is performed by autoclaving.

137. The method according to claim 133 wherein the kosmotropic salt is a sulphate.

138. The method according to claim 137 wherein the sulphate is ammonium or sodium sulphate.

139. The method according to claim 133 wherein the amount of kosmotropic salt is in the range of 0.2 M to 1.5 M.

140. The method according to claim 133 wherein the folded α-helical tertiary structure is a four helix bundle.

141. The method according to claim 126 wherein the polypeptide or protein comprises at least two α-helical peptides linked by a linking sequence of 3 to 11 amino acid residues, wherein each α-helical peptide comprises a sequence of amino acid residues:
(a b c d d′ e f g)ₙ
wherein n is an integer from 2 to 12;
amino acid residues a and d are hydrophobic amino acid residues;
amino acid residue d′ is absent or is a hydrophobic amino acid residue;
at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
amino acid residue g is any amino acid residue.

142. The method according to claim 141 wherein the linking sequence comprises a cleavable bond and the method further comprises the step of cleaving the cleavable bond.

143. The method according to claim 141 wherein each sequence (a b c d d′ e f g) in the α-helical peptide comprises at least one glutamine or asparagine residue and no net charge or each sequence (a b c d d′ e f g) comprises at least one glutamine or asparagine residue and one negative charge and the method further comprises the step of deamidating the glutamine and/or asparagine residues.

144. The method according to claim 126 wherein the polynucleotide sequence further encodes a second protein, polypeptide or peptide and a cleavable linker operably linked with the nucleotide sequence encoding the polypeptide or protein, such that step ii) expresses a fusion protein comprising the second protein, polypeptide or peptide cleavably linked to the polypeptide or protein.

145. The method according to claim 144 further comprising the step of cleaving the cleavable linker.

146. The method according to claim 144 wherein the second protein, polypeptide or peptide is an antimicrobial peptide or a protease, lipase, amylase or cellulase enzyme or a peptide for use in the manufacture of metallic or semiconductor nanostructures.

147. The method according to claim 144 wherein the cleavable linker between the polypeptide or protein and the second protein, polypeptide or peptide is cleaved by an enzyme at a faster rate than the rate of non-specific cleavage of the protein or polypeptide.

148. A composition comprising a polypeptide or protein according to claim 76 and an α-helical peptide comprising the amino acid sequence:
X₁-(a b c d d′ e f g)ₙ-X₂
wherein n is an integer from 2 to 12;
amino acid residues a and d are hydrophobic amino acid residues;
amino acid residue d′ is absent or is a hydrophobic amino acid residue;
at least one of amino acid residues b and c and at least one of amino acid residues e and f are hydrophilic amino acid residues, the other of amino acid residues b and c and e and f are any amino acid residue, provided that amino acid residues b and c are not both charged amino acid residues with the same charge and amino acid residues e and f are not both charged amino acid residues with the same charge;
amino acid residue g is any amino acid residue;
wherein:
i) each α-helical peptide comprises at least one stimuli-responsive amino acid residue; or
ii) each sequence (a b c d d′ e f g) in the α-helical peptide comprises at least one glutamine or asparagine residue and no net charge or each sequence (a b c d d′ e f g) comprises at least one glutamine or asparagine residue and one negative charge;
X₁ and X₂ are each independently absent or are amino acid residues from a cleavable linking sequence of the polypeptide or protein of claim 1 and wherein the number of amino acid residues in X₁ and X₂ is 3 to 11 amino acid residues.

149. A composition according to claim 148 wherein the polypeptide is a polypeptide of SEQ ID NO:1.

150. A composition according to claim 148 wherein the α-helical peptide is a peptide of SEQ ID NO:12.


SEQUENCE LISTING (31 sequences)

SEQ ID NO: 1 - protein, 98 aa, Artificial Sequence
  Synthetic
MDPSMKQLADSLHQLARQVSRLEHADPSMKQLADSLHQLARQVSRLEHADPSMKQLADSLHQLARQVSRLEHADPSMKQLADSLHQLARQVSRLEHAD

SEQ ID NO: 2 - protein, 50 aa, Artificial Sequence
  Synthetic
MDPSMKQLADSLHQLARQVSRLEHADPSMKQLADSLHQLARQVSRLEHAD

SEQ ID NO: 3 - protein, 98 aa, Artificial Sequence
  Synthetic
MDPSAKSLAESLHSLARSVSRLEHADPSAKSLAESLHSLARSVSRLEHADPSAKSLAESLHSLARSVSRLEHADPSAKSLAESLHSLARSVSRLEHAD

SEQ ID NO: 4 - protein, 102 aa, Artificial Sequence
  Synthetic
MDPSAKSVAESLHSLARSVSRLVEHADPSAKSVAESLHSLARSVSRLVEHADPSAKSVAESLHSLARSVSRLVEHADPSAKSVAESLHSLARSVSRLVEHAD

SEQ ID NO: 5 - protein, 102 aa, Artificial Sequence
  Synthetic
MDPSAHSVAESLHSLARSVSRLVEHADPSAHSVAESLHSLARSVSRLVEHADPSAHSVAESLHSLARSVSRLVEHADPSAHSVAESLHSLARSVSRLVEHAD

SEQ ID NO: 6 - protein, 102 aa, Artificial Sequence
  Synthetic
MDPSAHSVAKSLHSLARSVSRLVSHADPSAHSVAKSLHSLARSVSRLVSHADPSAHSVAKSLHSLARSVSRLVSHADPSAHSVAKSLHSLARSVSRLVSHAD

SEQ ID NO: 7 - protein, 102 aa, Artificial Sequence
  Synthetic
MDPSAHSVAESLHSLAESVSELVSHADPSAHSVAESLHSLAESVSELVSHADPSAHSVAESLHSLAESVSELVSHADPSAHSVAESLHSLAESVSELVSHAD

SEQ ID NO: 8 - protein, 102 aa, Artificial Sequence
  Synthetic
MDPSAQSVAQSLAQLAQSVSQLVSQADPSAQSVAQSLAQLAQSVSQLVSQADPSAQSVAQSLAQLAQSVSQLVSQADPSAQSVAQSLAQLAQSVSQLVSQAD

SEQ ID NO: 9 - protein, 102 aa, Artificial Sequence
  Synthetic
MDPSANSVANSLANLANSVSNLVSNADPSANSVANSLANLANSVSNLVSNADPSANSVANSLANLANSVSNLVSNADPSANSVANSLANLANSVSNLVSNAD

SEQ ID NO: 10 - protein, 102 aa, Artificial Sequence
  Synthetic
MDPSAQSVAESLAQLAESVSELVSQADPSAQSVAESLAQLAESVSELVSQADPSAQSVAESLAQLAESVSELVSQADPSAQSVAESLAQLAESVSELVSQAD

SEQ ID NO: 11 - protein, 102 aa, Artificial Sequence
  Synthetic
MDPSANSVAESLANLAESVSELVSNADPSANSVAESLANLAESVSELVSNADPSANSVAESLANLAESVSELVSNADPSANSVAESLANLAESVSELVSNAD

SEQ ID NO: 12 - protein, 24 aa, Artificial Sequence
  Synthetic
PSMKQLADSLHQLARQVSRLEHAD

SEQ ID NO: 13 - protein, 24 aa, Artificial Sequence
  Synthetic
PSMKELADSLHELAREVSRLEHAD

SEQ ID NO: 14 - protein, 24 aa, Artificial Sequence
  Synthetic
PSMKELADSLHQLARQVSRLEHAD

SEQ ID NO: 15 - protein, 24 aa, Artificial Sequence
  Synthetic
PSMKQLADSLHELARQVSRLEHAD

SEQ ID NO: 16 - protein, 24 aa, Artificial Sequence
  Synthetic
PSMKQLADSLHQLARQVSRLEHAD

SEQ ID NO: 17 - protein, 24 aa, Artificial Sequence
  Synthetic
PSMKELADSLHELARQVSRLEHAD

SEQ ID NO: 18 - protein, 24 aa, Artificial Sequence
  Synthetic
PSMKELADSLHQLAREVSRLEHAD

SEQ ID NO: 19 - protein, 24 aa, Artificial Sequence
  Synthetic
PSMKQLADSLHELAREVSRLEHAD

SEQ ID NO: 20 - protein, 24 aa, Artificial Sequence
  Synthetic
PSAKSLAESLHSLARSVSRLEHAD

SEQ ID NO: 21 - protein, 25 aa, Artificial Sequence
  Synthetic
PSAKSVAESLHSLARSVSRLVEHAD

SEQ ID NO: 22 - protein, 25 aa, Artificial Sequence
  Synthetic
PSAHSVAESLHSLARSVSRLVEHAD

SEQ ID NO: 23 - protein, 25 aa, Artificial Sequence
  Synthetic
PSAHSVAKSLHSLARSVSRLVSHAD

SEQ ID NO: 24 - protein, 25 aa, Artificial Sequence
  Synthetic
PSAHSVAESLHSLAESVSELVSHAD

SEQ ID NO: 25 - protein, 25 aa, Artificial Sequence
  Synthetic
PSAQSVAQSLAQLAQSVSQLVSQAD

SEQ ID NO: 26 - protein, 25 aa, Artificial Sequence
  Synthetic
PSAESVAESLAELAESVSELVSEAD

SEQ ID NO: 27 - protein, 25 aa, Artificial Sequence
  Synthetic
PSANSVANSLANLANSVSNLVSNAD

SEQ ID NO: 28 - protein, 25 aa, Artificial Sequence
  Synthetic
PSADSVADSLADLADSVSPLVSDAD

SEQ ID NO: 29 - protein, 25 aa, Artificial Sequence
  Synthetic
PSAQSVAESLAQLAESVSELVSQAD

SEQ ID NO: 30 - protein, 25 aa, Artificial Sequence
  Synthetic
PSAESVAESLAELAESVSELVSEAD

SEQ ID NO: 31 - protein, 25 aa, Artificial Sequence
  Synthetic
PSANSVAESLANLAESVSELVSNAD